(19) United States
(12) Patent Application Publication
(10) Pub. No.: US 2024/0109896 A1
(43) Pub. Date: Apr. 4, 2024

(54) FGFR KINASE INHIBITOR AND USE THEREOF

(71) Applicant: YA THERAPEUTICS INC, Shanghai (CN)

(72) Inventors: Yonghong LIANG, Shanghai (CN); Zhiyong XU, Shanghai (CN); Zhaosen ZENG, Shanghai (CN); Wenguang YAN, Shanghai (CN); Fangjun XIONG, Shanghai (CN)

(21) Appl. No.: 18/261,899

(22) PCT Filed: Dec. 27, 2021
(86) PCT No.: PCT/CN2021/141768

§ 371 Date: Jul. 18, 2023

(30) Foreign Application Priority Data
Feb. 3, 2021 (CN) 202110152502.6
Jun. 2, 2021 (CN) 202110615730.2
Nov. 19, 2021 (CN) 202111373638.6

(51) Int. Cl. C07D 487/04; A61P 35/00; C07D 403/04; C07D 403/12; C07D 409/14

(52) U.S. Cl. CPC C07D 487/04; A61P 35/00; C07D 403/04; C07D 403/12; C07D 409/14

(57) ABSTRACT

Disclosed are a heterocyclic compound used as a FGFR (Fibroblast Growth Factor Receptor) inhibitor, a preparation method thereof, and a pharmaceutical application thereof. Specifically, the present disclosure relates to a compound represented by general formula I and a pharmaceutically acceptable salt thereof, a pharmaceutical composition comprising the found and/or the pharmaceutically acceptable salt, and a use of the compound or the pharmaceutically acceptable salt in treating or preventing disorders related to FGFR kinase, specifically tumor drugs, and the compound is a class of heterocyclic compound, and at the same time the present disclosure provides a preparation method for the pharmaceutical composition of the compound and the pharmaceutically acceptable salt. Definition of substituents in general formula I is identified with that in the description.

### CROSS-REFERENCE TO RELATED APPLICATION

This application claims priority to International Application Nos. CN20211001525026, filed on Feb. 3, 2021, International Application Nos. CN2021106157302, filed on Jun. 2, 2021, and International Application Nos. CN2021113736386, filed on Nov. 19, 2021, and the content of each of which is incorporated herein by reference in its entirety.

### TECHNICAL FIELD

The present disclosure provides a compound acting as a Fibroblast Growth Factor Receptor Inhibitors (FGFR) and therefore useful for the treatment of diseases treatable by inhibition of FGFR, and further provides a pharmaceutical composition containing such compound and a method for preparing such compound.

### BACKGROUND

Fibroblast Growth Factor Receptor (FGFR) belongs to receptor tyrosine kinases. FGFR mainly comprises four members: FGFR1, FGFR2, FGFR3 and FGFR4. FGFRs participate and regulate cell proliferation, migration, apotosis, angiogenesis and many other processes. For their wide functions, FGFRs and other RTKs are strictly regulated under normal conditions. In tumors, such as liver cancer, bladder cancer, lung cancer, breast cancer and prostate caner, FGFR activation mutation or ligand/receptor over-expression would cause their continuous constitutive activation. The binding of an FGF to an FGFR leads to receptor dimerization and transphosphorylation of tyrosine kinase domains (Dieci, M. V, et aL, Cancer Discov. 2013; 3:264-279; Korc, N., and Friesel, R. E., Curr. Cancer Drug Targets 2009; 5:639-651). Activation of downstream signaling occurs via the intracellular receptor substrate FGFR substrate 2 (FRS2) and phospholipase Cy (PLC-γ), leading to subsequent upregulation of RAS/mitogen-activated protein kinase (MAPK) and phosphoinositide kinase (PI3K)/AKT signaling pathways. Other pathways can be activated, including STAT-dependent signaling (Turner, N., Grose, R., Nat. Ref. Cancer 2010; 10:116-129; Brooks, N. S., et al., Clin Cancer Res. 2012; 18:1855-1862; Dienstmann, R., et al., Ann. Oncol. 2014; 25:552-563).

FGFR signaling components are frequently altered in human cancer, and several preclinical models have provided compelling evidence for the oncogenic potential of aberrant FGFR signaling in carcinogenesis, thereby validating FGFR signaling as an attractive target for cancer treatment.

Recently, industry and academia have made great efforts in the research of small molecule FGFR inhibitors. Some FGFR inhibitors such as erdatinib, infilgratinib, and pemigartinib, as well as some other small molecule inhibitors have been reported: WO2011071821, WO2011135376, WO2014007951, WO2015008839, WO2015008844, WO2014011900, WO2015061572, WO2015108992, WO2017215485, WO2020168237, WO2018028438, WO2018049781, WO2019034075, WO2018121650, WO2020231990, WO2021146424.

Although some FGFR inhibitors have entered the clinical and preclinical development process, the poor selectivity and inhibitory effects on other kinases such as c-kit and PDGFRa brings some concern. Therefore, it is significant to develop inhibitors targeting FGFR selectivity in the clinical treatment of diseases with elevated FGF or FGFR activity.

### SUMMARY

A compound represented by general formula (I), a stereoisomer thereof, a pharmaceutical salt, a polymorph or an isomer, where the structure of the compound represented by the general formula (I) is as follows

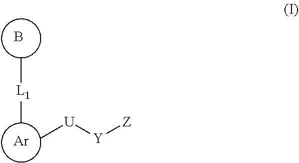

In the formula,
Each ring B is a benzyl ring or a 5-10 membered heteroaromatic ring, and the above benzene ring and heteroaromatic ring can be optionally substituted one or more G¹;
Each L₁ is independently selected from bonds, —C₁₋₄ alkyl-, —C₂₋₄ alkenyl-, —C₂₋₄ alkyne-;
Each aromatic ring Ar is 6-10 membered heteroaromatic ring, and the benzyl ring and heteroaromatic ring described above can be optionally substituted with one or more R¹.
Each R¹ is independently selected from H, D, cyano, halogen, C₁₋₆ alkyl, C₃₋₆ cycloalkyl, 3-6 membered heterocyclyl, —OR², —NR²R³, —C(O)NR²R³, wherein the alkyl, cycloalkyl or heterocyclyl are optionally cyano, halogen, —OR⁴, —NR⁴R⁵, C₁₋₆ alkyl, C₃₋₆ cycloalkyl or 3-6 membered heterocyclyl;
Each U is independently selected from —C₀₋₄ alkyl-, —CR⁶R⁷—, —C₁₋₂ alkyl (R⁶)(OH)—, —C(O)—, —CR⁶R⁷O—, —OCR⁶R⁷—, —SCR⁶R⁷—, —CR⁶R⁷S—, —NR⁶—, —NR⁶C(O)—, —C(O)NR⁶—, —NR⁶C(O)NR⁷—, —CF₂—, —O—, —S—, —S(O)ₘ—, —NR⁶S(O)₂—, —S(O)₂NR⁶—;
Each Y is absent or selected from C₃₋₈ cyclo alkyl, 3-8-membered heterocyclic alkyl, 5-12 thick alkyl, 5-12 thick heterocyclyl, 5-12 membered spirocyclyl, 5-12 membered spiroheterocyclyl, aryl or heteroaryl; the 3-8 heterocycloalkyl, 5-12 membered thick heterocyclyl, 5-12 membered spiroheterocyclyl group or heteroaryl independently comprises 1, 2, 3, or 4 heteroatoms selected from N, O, or S at each occurrence; and the cycloalkyl, heterocyclyalkyl, spirocyclyl, polycycle, heteropolycyclyl, heterospirocyclyl, aryl, or heteroaryl is optionally substituted with one or more G²;
Each Z is independently selected from cyano, —NR⁸CN,

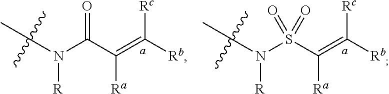

Bond a is a double bond or a triple bond;
When a is a double bond, each of R^{a}, R^{b} and R^{c} is independently selected from H, D, cyano, halogen, C₁₋₆ alkyl, C₃₋₆ cycloalkyl or 3-6 membered heterocyclyl, and the alkyl, cycloalkyl and heterocyclyl are optionally substituted with one or more G³;
Each of R^{a} and R^{b} or R^{b} and R^{c} optionally forms an optional 3-6 membered heterocyclyl with carbon atoms attached to them;
When a is a triple bond, R^{a} and R^{c} are absent, R^{b} is independently selected from H, D and cyano; and the halogen, C₁₋₆ alkyl, C₃₋₆ cycloalkyl or 3-6 heterocycloalkyl is substituted with one or more G⁴;
Each R⁸ is independently selected from H, D, C₁₋₆ alkyl, C₃₋₆ cycloalkyl or 3-6-membered heterocyclyl, and the alkyl, cycloalkyl and heterocyclyl are optionally substituted with one or more G⁵;
Each of G¹, G², G³, G⁴ and G⁵ is independently selected from H, D, cyano, halogen, C₁₋₆ alkyl, C₂₋₆ alkenyl, C₂₋₆ alkyl, C₃₋₈ cycloalkyl or 3-8-membered heterocyclyl, C₆₋₁₀ aryl, 5-10 heteroaryl, —OR⁹, —OC(O)NR⁹R¹⁰, —C(O)OR⁹, —C(O)NR⁹R¹⁰, —C(O)R⁹, —NR⁹R¹⁰, —NR⁹C(O)R¹⁰, —NR⁹C(O)NR¹⁰R¹¹, —S(O)ₘR⁹ or —NR⁹S(O)ₘR¹⁰; and the alkyl, alkenyl, alkynyl, cycloalkyl, heterocyclylalkyl, aryl and heteroaryl are optionally substituted with substituents of one or more cyano, halogen, C₁₋₆ alkyl, C₂₋₆ alkenyl, C₂₋₆ alkyl, C₃₋₈ cycloalkyl or 3-8 membered heterocyle, C₆₋₁₀ aryl, 5-10 membered heteroaryl, —OR¹², —OC(O)NR¹²R¹³, —C(O)OR¹², —C(O)NR¹²R¹³, —C(O)R¹², —NR¹²R¹³, —NR¹²C(O)R¹³, —NR¹²C(O)NR¹³R¹⁴, —S(O)ₘR¹² or —NR¹²S(O)ₘR¹³.
Each of R³, R⁴, R⁵, R⁶, R⁷, R⁸, R⁹, R¹¹, R¹², R¹³ and R¹⁴ is independently selected from H, D, cyano, halogen, C₁₋₆ alkyl, C₃₋₈ cycloalkyl or 3-8 membered monocyclic heterocycyl, monocyclic heteroaryl or phenyl, and m is 1 or 2.

In some implementation modes, the compound of general formula (I), the pharmaceutically acceptable salt thereof, or the stereo isomer is further shown in general formula IIa:

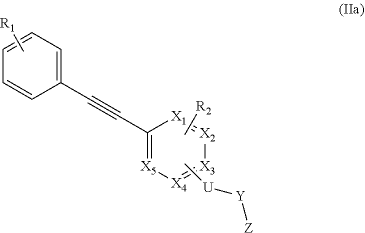

In the formula,
Each of X₁, X₂, X₃, X₄, X₅ is independently selected from CR¹ or N, and at least one of X₁, X₂, X₃, X₄, X₅ is N;
Each R¹ is independently selected from H, D, cyano, halogen, C₁₋₆ alkyl, C₃₋₆ cycloalkyl, 3-6 membered heteroalkyl, —OR², —NR²R³, —C(O)NR²R³, and the alkyl, cycloalkyl or heteroalkyl can be optionally substituted with cyano, halogen, —OR⁴, —NR⁴R⁵, C₁₋₆ alkyl, C₃₋₆ cycloalkyl or 3-6 membered heterocyclyl;
Each ring B is a benzene ring or a 5-6 membered heteroaryl, and the aryl and heteroaryl described above are optionally substituted with one or more G¹;
Each U is independently selected from —C₀₋₄ alkyl-, —CR⁶R⁷—, —C₁₋₂alkyl(R⁶)(OH)—, —C(O)—, —CR⁶R⁷O—, —OCR⁶R⁷—, —SCR⁶R⁷—, —CR⁶R⁷S—, —NR⁶—, —NR⁶C(O)—, —C(O)NR⁶—, —NR⁶C(O)NR⁷—, —CF₂—, —O—, —S—, —S(O)ₘ—, —NR⁶S(O)₂—, —S(O)₂NR⁶—;
Each Y is absent or selected from C₃₋₈ cycloalkyl, 3-8-membered heterocycloalkyl, 5-12 thick alkyl, 5-12 thick heterocyclyl, 5-12 membered spirocyclyl, 5-12 membered spirocyclyl, aryl or heteroaryl; 3-8 heterocycloalkyl, 5-12 membered thick heterocyclyl, 5-12 membered spiroheterocyclyl or heteroaryl independently comprises 1, 2, 3, or 4 heteroatoms selected from N, O, or S at each occurrence, the cycloalkyl, heterocycloalkyl, spirocyclyl, polycyclic, heteropolycyclyl, heterospirocyclyl, aryl or heteroaryl is optionally substituted with one or more G²;
Each Z is independently selected from cyano, —NR⁸CN,

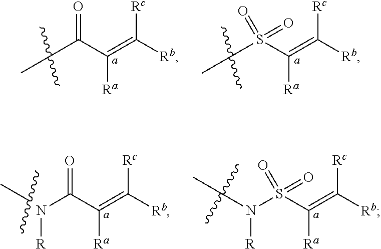

Bond a is a double or a triple bond;
When a is a double bond, each of R^{a}, R^{b} and R^{c} is independently selected from H, D, cyano, halogen, C₁₋₆ alkyl, C₃₋₆ cycloalkyl or 3-6 membered heterocyclyl, and the alkyl, cycloalkyl and heterocyclyl are optionally substituted with one or more G³.
Each of R^{a} and R^{b} or R^{b} and R^{c} optionally forms an optional 3-6-membered ring containing heteroatoms with carbon atoms attached to them;
When bond a is a triple bond, R^{a} and R^{c} are absent, each R^{b} is independently selected from H, D and cyano, and the halogen, C₁₋₆ alkyl, C₃₋₆ cycloalkyl or 3-6 membered heterocyclyl is substituted with one or more G⁴;
Each R⁸ is independently selected from H, D, C₁₋₆ alkyl, C₃₋₆ cyclocyclyl or 3-6 membered heterocyclyl. and the alkyl, cycloalkyl and heterocyclyl are optionally substituted with 1 or more G⁵;
Each of G¹, G², G³, G⁴ and G⁵ is independently selected from H, D, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR⁹, —OC(O)NR⁹R¹⁰, —C(O)OR⁹, —C(O)NR⁹R¹⁰, —C(O)R⁹, —NR⁹R¹⁰, —NR⁹C(O)R¹⁰, —NR⁹C(O)NR¹⁰R¹¹, —S(O)ₘR⁹ or —NR⁹S(O)ₘR¹⁰, and the alkyl, alkenyl, alkynyl, cycloalkyl, heteroalkyl, aryl and heteroaryl are optionally substituted with substituents of one or more cyano, halogen, C₁₋₆ alkyl, C₂₋₆ alkenyl group, C₂₋₆ alkyl, C₃₋₈ cycloalkyl or 3-8 membered heterocyclyl, C₆₋₁₀ aryl, 5-10 membered heteroaryl, —OR¹², —OC(O)NR¹²R¹³, —C(O)OR¹², —C(O)NR¹²R¹³, —C(O)R¹², —NR¹²R¹³, —NR¹²C(O)R¹³, —NR¹²C(O)NR¹³R¹⁴, —S(O)ₘR¹² or —NR¹²S(O)ₘR¹³.
Each of R³, R⁴, R⁵, R⁶, R⁷, R⁸, R⁹, R¹¹, R¹², R¹³ and R¹⁴ is independently selected from H, D, cyano, halogen, C₁₋₆ alkyl, C₃₋₈ cycloalkyl or 3-8 membered monocyclic heterocyclyl, monocyclic heteroaryl or phenyl; and
m is 1 or 2.

In some implementation modes, the compound of general formula (I), the pharmaceutically acceptable salt thereof, or the stereo isomer is further shown in general formula IId:

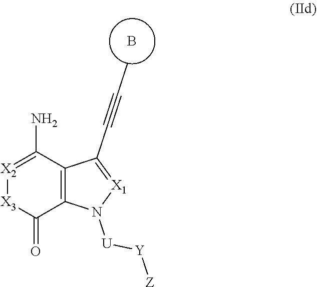

In the formula:
X₁, X₂, X₃ can be independently selected from N, CR¹;
Each R¹ is independently selected from H, D, cyano, halogen, C₁₋₆ alkyl, C₃₋₆ cycloalkyl, 3-6-membered heteroalkyl, —OR², —NR²R³, —C(O)NR²R³, and the alkyl, formula or heteroalkyl is optionally substituted with cyano, halogen, —OR⁴, —NR⁴R⁵, C₁₋₆ alkyl, C₃₋₆ cycloalkyl or 3-6 hetero cycloalkyl;
Each ring B is a benzyl or a 5-6 heteroaryl, and the benzyl and 5-6 heteroaryl described above are optionally substituted with one or more G¹;
Each U is independently selected from —C₀₋₄ alkyl-, —CR⁶R⁷—, —C₁₋₂ alkyl, (R⁶)(OH)—, —C(O)—, —CR⁶R⁷O—, —OCR⁶R⁷—, —SCR⁶R⁷—, —CR⁶R⁷S—, —NR⁶—, —NR⁶C(O)—, —C(O)NR⁶—, —NR⁶C(O)NR⁷—, —CF₂—, —O—, —S—, —S(O)ₘ—, —NR⁶S(O)₂—, —S(O)₂NR⁶—;
Each Y is absent or selected from C₃₋₈ cycloalkyl, 3-8-membered heterocycloalkyl, 5-12 thick alkyl, 5-12 thick heterocyclyl, 5-12 membered spirocyclyl, 5-12 membered spiroheterocyclyl, aryl or heteroaryl; the 3-8 membered heterocycloalkyl, 5-12 membered thick heterocyclyl, 5-12 spiro heterocyclyl or heteroaryl independently comprises 1, 2, 3, or 4 heteroatoms selected from N, O, or S at each occurrence, and the cycloalkyl group, heteroalkyl, spirocyclyl, polycycle, polycycle, spirocyclyl, aryl, or heteroaryl is optionally substituted with one or more G²;
Each Z is independently selected from cyano, —NR⁸CN

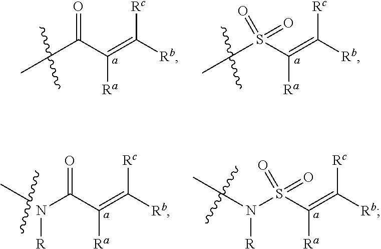

Bond a is a double or a triple bond;
When a is a double bond, each of R^{a}, R^{b} and R^{c} is independently selected from H, D, cyano, halogen, C₁₋₆ alkyl, C₃₋₆ cycloalkyl or 3-6 heterocyclyl, and the alkyl, cycloalkyl and heterocyclyl are optionally substituted one or more G³;
Each of R^{a} and R^{b} or R^{b} and R^{c} optionally forms an optional 3-6-membered ring containing heteroatoms with carbon atoms attached to them;
When a is a triplet bond R^{a} and R^{c} are absent. Each R^{b} is independently selected for H, D and cyano, and the halogen, C₁₋₆ alkyl, C₃₋₆ cycloalkyl or 3-6 heterocyclyl is substituted with one or more G⁴.
Each R⁸ is independently selected from H, D, C₁₋₆ alkyl, C₃₋₆ naphthenic or 3-6-membered heterocyclic groups, and the alkyl, naphthenic and heterocyclic groups are optionally substituted with one or more G⁵;
Each of G¹, G², G³, G⁴ and G⁵ is independently selected from H, D, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR⁹, —OC(O)NR⁹R¹⁰, —C(O)OR⁹, —C(O)NR⁹R¹⁰, —C(O)R⁹, —NR⁹R¹⁰, —NR⁹C(O)R¹⁰, —NR⁹C(O)NR¹⁰R¹¹, —S(O)ₘR⁹ or —NR⁹S(O)ₘR¹⁰; the alkyl, alkenyl, alkynyl, cycloalkyl, heteroalkyl, aryl or heteroaryl is optionally substituted with substituents of one or more cyano, halogen, C₁₋₆ alkyl, C₂₋₆ alkenyl, C₂₋₆ alkynyl, C₃₋₈ heteroalkyl or 3-8 membered heterocyclyl, C₆₋₁₀ aryl, 5-10 membered heteroaryl, —OR¹², —OC(O)NR¹²R¹³, —C(O)OR¹², —C(O)NR¹²R¹³, —C(O)R¹², —NR¹²R¹³, —NR¹²C(O)R¹³, —NR¹²C(O)NR¹³R¹⁴, —S(O)ₘR¹² or —NR¹²S(O)ₘR¹³.
Each of R³, R⁴, R⁵, R⁶, R⁷, R⁸, R⁹, R¹¹, R¹², R¹³ and R¹⁴ is independently selected from H, D, cyano, halogen, C₁₋₆ alkyl, C₃₋₈ cycloalkyl or 3-8 membered monocyclic heterocyclyl, monocyclic heteroaryl or phenyl; and
m is 1 or 2.

In some implementation modes, the compound of general formula (I), the pharmaceutically acceptable salt thereof, or the stereo isomer is further shown in general formula IIe:

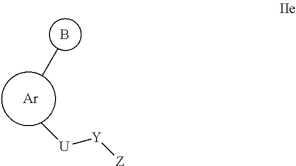

In the formula:
The ring Ar is a 5-10 membered heteroaryl, and the above 5-10 membered aryl is optionally substituted with one or more G¹;
Ring B is independently selected from 5-14 heteroaryl and 5-14 heteroaryl containing 1-3 heteroatoms selected from S, O, N and Se, and the 5-14 heteroaryl described above is substituted with one or more G²;
Each U is dependently selected from —C₀₋₄ alkyl-, —CR⁷R⁸—, —C₁₋₂alkyl (R⁷)(OH)—, —C(O)—, —CR⁷R⁸O—, —OCR⁷R⁸—, —SCR⁷R⁸—, —CR⁷R⁸S—, —NR⁷—, —NR⁷C(O)—, —C(O)NR⁷—, —NR⁷C(O)NR⁸—, —CF₂—, —O—, —S—, —S(O)ₘ—, —NR⁷S(O)₂—, —S(O)₂NR⁷—;
Each Y is absent or selected from C₃₋₈ cycloalkyl, 3-8-membered heterocycloalkyl, 5-12 thick alkyl, 5-12 thick heterocyclyl, 5-12 membered spirocyclyl, 5-12 membered spiroheterocyclyl, aryl or heteroaryl, and the cycloalkyl, heterocyclyl, spirocyclyl, thickcyclyl, thickheterocyclyl, spiroheterocyclyl, aryl or heteroaryl is substituted with one or more G³;
Z is independently selected from cyano, NR⁹CN,

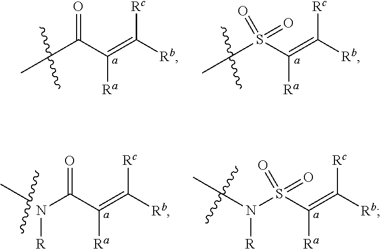

Bond a is a double bond or a triple bond;
When a is a double bond, each of R^{a}, R^{b} and R^{c} is independently selected from H, D, cyano, halogen, C₁₋₆ alkyl, C₃₋₆ cycloalkyl or 3-6 heterocyclyl, and the alkyl, cycloalkyl and heterocyclyl are optionally substituted one or more G⁴;
R^{a} and R^{b} or R^{b} and R^{c} optionally form an optional 3-6-membered ring containing heteroatoms with carbon atoms attached to them;
When a is a triplet bond, R^{a} and R^{c} are absent, each R^{b} is independently selected for H, D and cyano, and the halogen, C₁₋₆ alkyl, C₃₋₆ cycloalkyl or 3-6 heterocyclyl is substituted with one or more G⁵.
R⁹ is independently selected from H, D, C₁₋₆ alkyl, C₃₋₆ cycloalkyl or 3-6-membered heterocyclyl, and the alkyl cycloalkyl and heterocyclyl are optionally substituted with one or more G⁶;
Each of G¹, G², G³, G⁴ and G⁵ is independently selected from H, D, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR¹⁰, —OC(O)NR¹⁰R¹¹, —C(O)OR¹⁰, —C(O)NR¹⁰R¹¹, —C(O)R¹⁰, —NR¹⁰R¹¹, —NR¹⁰C(O)R¹¹, —NR¹⁰C(O)NR¹¹R¹², —S(O)ₘR¹⁰ or —NR¹⁰S(O)ₘR¹¹ and the alkyl, alkenyl, alkynyl, cycloalkyl, heteroalkyl, aryl or heteroaryl is optionally substituted with substituents of one or more cyano, halogen, C₁₋₆ alkyl, C₂₋₆ alkenyl, C₂₋₆ alkynyl, C₃₋₈ heteroalkyl or 3-8 membered heterocyclyl, C₆₋₁₀ aryl, 5-10 membered heteroaryl, —OR¹³, —OC(O)NR¹³R¹⁴, —C(O)OR¹³, —C(O)NR¹³R¹⁴, —C(O)R¹³, —NR¹³R¹⁴, —NR¹³C(O)R¹⁴, —NR¹³C(O)NR¹⁴R¹⁵, —S(O)ₘR¹³ or —NR¹⁴S(O)ₘR¹⁵
Each of R⁷, R⁸, R⁹, R¹⁰, R¹¹, R¹², R¹³, R¹⁴ and R¹⁵ is independently selected from H, D, cyano, halogen, C₁₋₆ alkyl, C₃₋₈ cycloalkyl or 3-8 membered monocyclic heterocycyl, monocyclic heteroaryl or phenyl; and
m is 1 or 2.

At each occurrence, each Ar is independently selected from

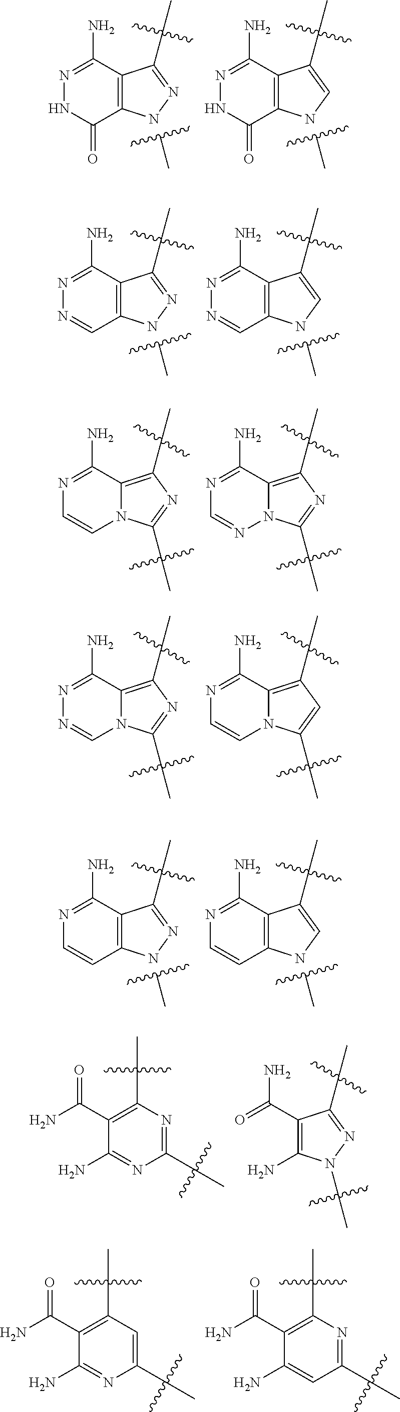

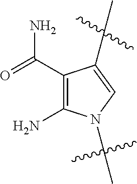 [SMILES: *c1cn(*)c(N)c1C(N)=O]

Each Ar is optionally substituted with one or more G¹ at each occurrence.
G¹ is independently selected from D, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR¹⁰, —OC(O)NR¹⁰R¹¹, —C(O)OR¹⁰, —C(O)NR¹⁰R¹¹, —C(O)R¹⁰, —NR¹⁰R¹¹, —NR¹⁰C(O)R¹¹, —NR¹⁰C(O)NR¹¹R¹², —S(O)ₘR¹⁰ or —NR¹⁰S(O)ₘR¹¹, and the alkyl, alkenyl, alkynyl, cycloalkyl, heteroalkyl, aryl, heteroaryl is optionally substituted with substituents of one or more cyano, halogen, C₁₋₆ alkyl, C₂₋₆ alkenyl, C₂₋₆ alkynyl, C₃₋₈ heteroalkyl or 3-8 membered heterocyclyl, C₆₋₁₀ aryl, 5-10 membered heteroaryl, —OR¹³, —OC(O)NR¹³R¹⁴, —C(O)OR¹³, —C(O)NR¹³R¹⁴, —C(O)R¹³, —NR¹³R¹⁴, —NR¹³C(O)R¹⁴, —NR¹³C(O)NR¹⁴R¹⁵, —S(O)ₘR¹³ or —NR¹³S(O)ₘR¹⁴.
Each of R¹⁰, R¹¹, R¹², R¹³, R¹⁴ and R¹⁵ is independently selected from H, D, cyano, halogen, C₁₋₆ alkyl, C₃₋₈ cycloalkyl or 3-8 membered monocyclic heterocycyl, monocyclic heteroaryl or phenyl; and
m is 1 or 2.

In some implementation modes, the compound of general formula (I), its isomers, solvate or precursors, or their pharmaceutically acceptable salts are selected from the following compounds, isomers, solvates or precursors, or their pharmaceutically acceptable salts:

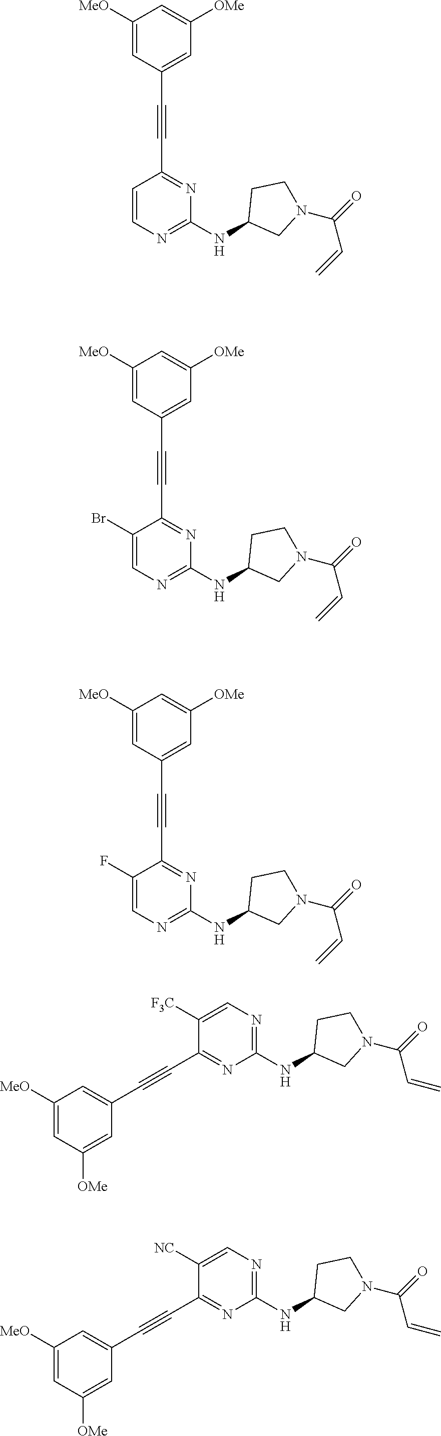

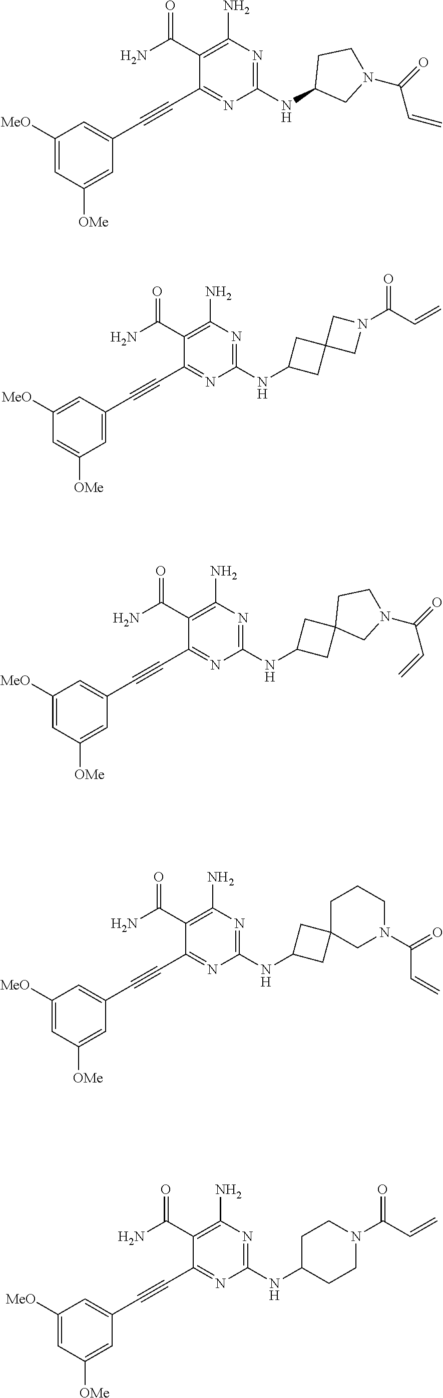

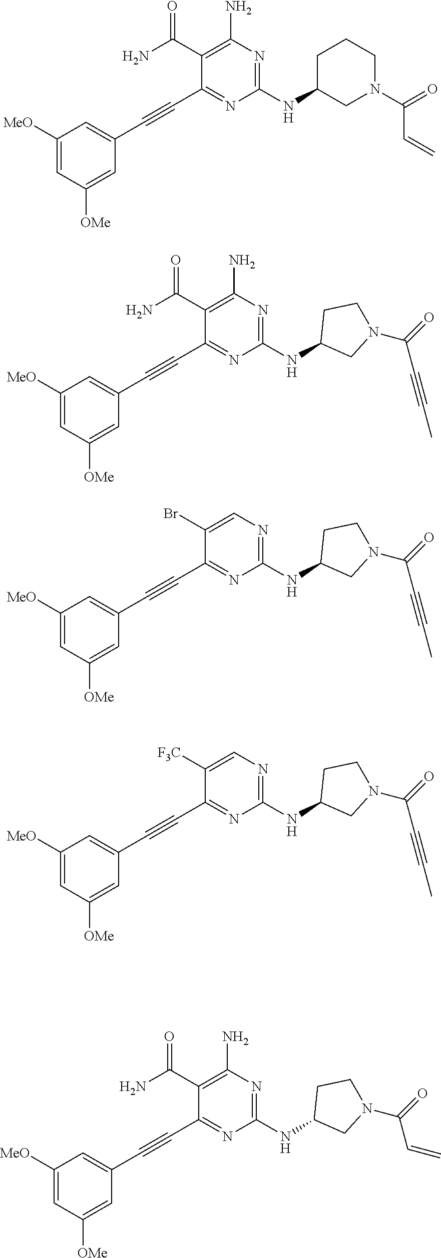

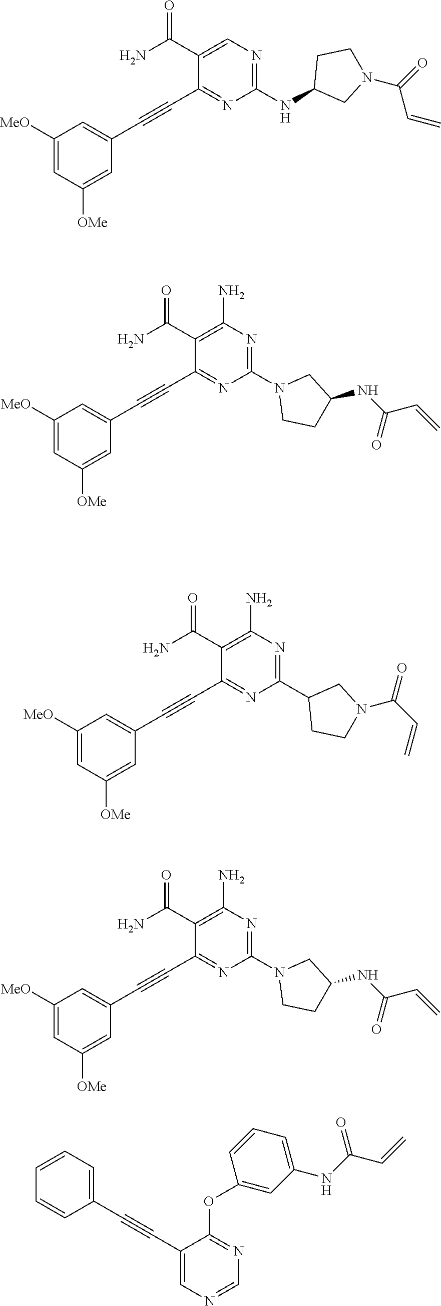

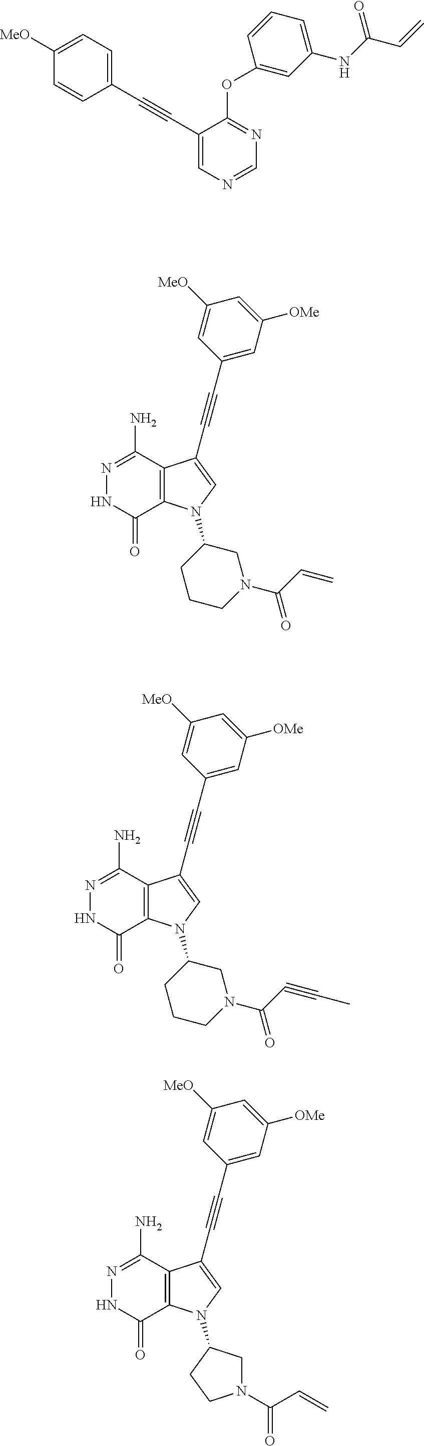

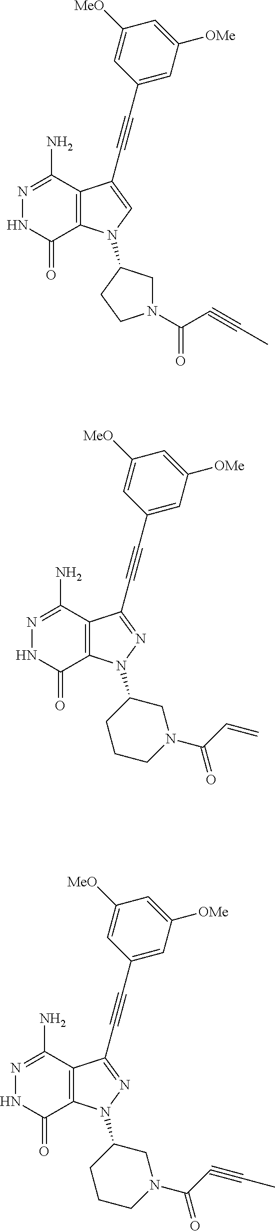

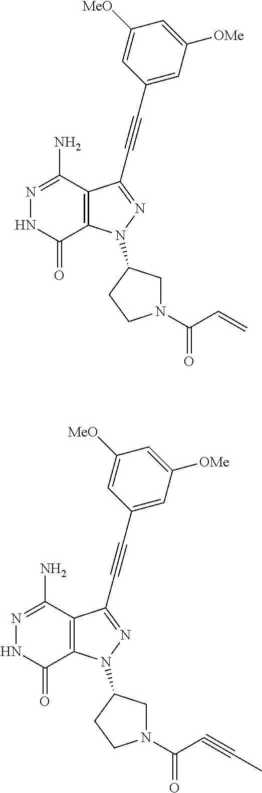

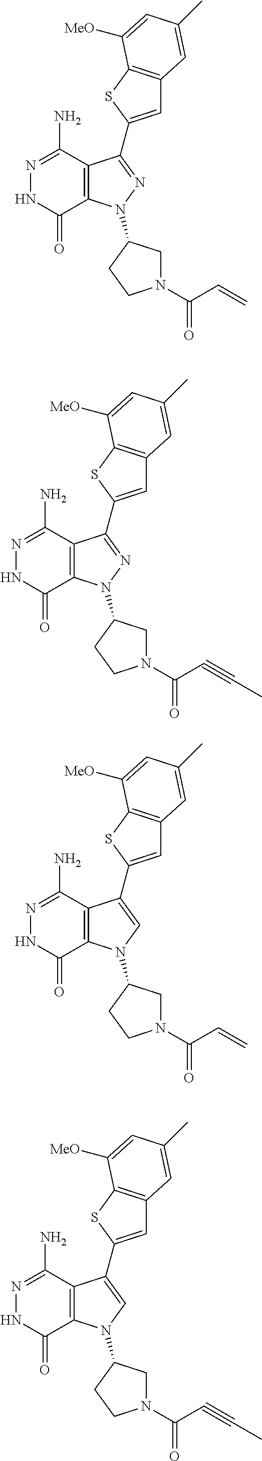

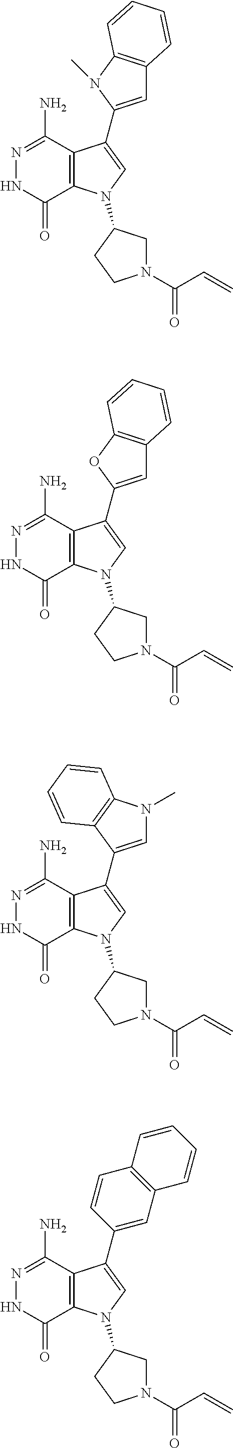

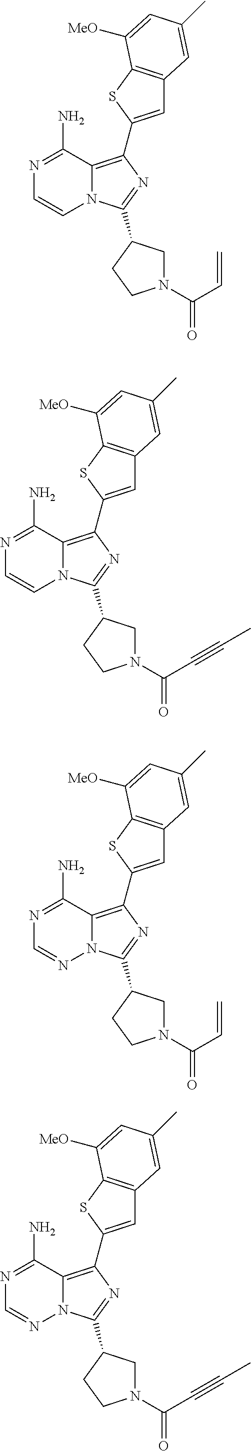

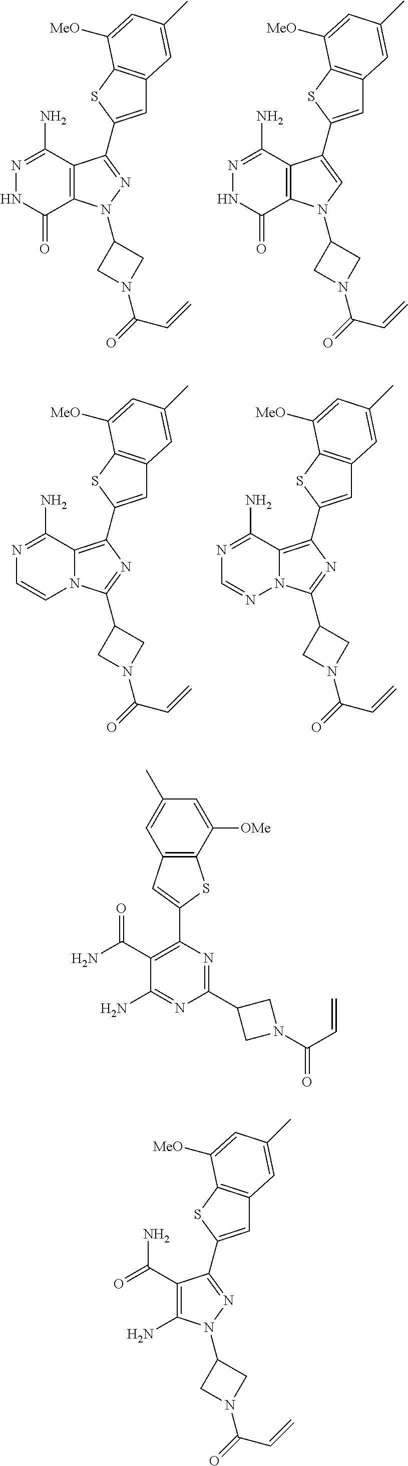

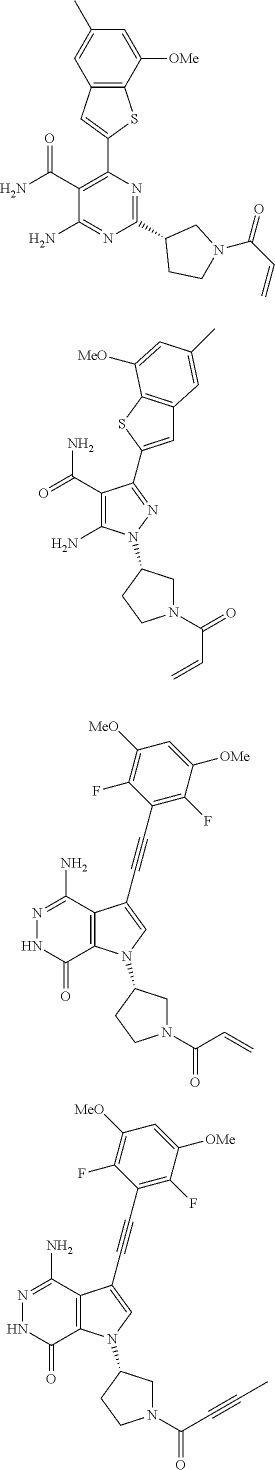

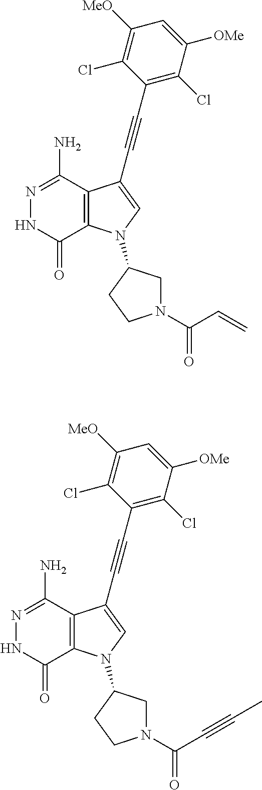

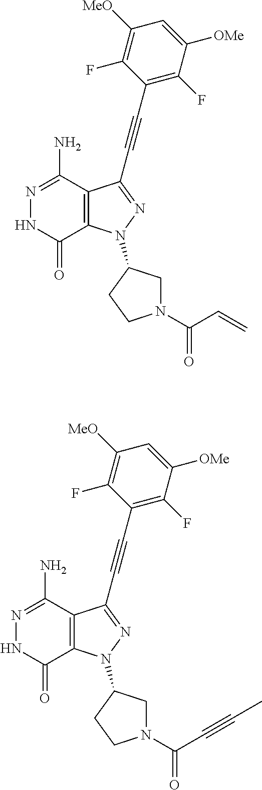

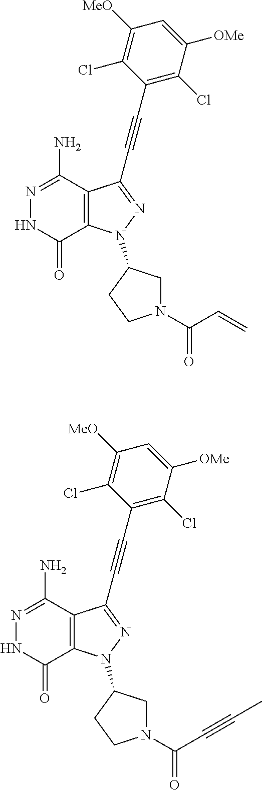

or their prodrugs, stable isotope derivatives, pharmaceutical acceptable salts, isomers and isomer mixtures and forms.

Compounds of the present disclosure can effectively inhibit the activity of FGFR1, FGFR2, FGFR3 or FGFR4, which inhibits the IC₅₀ of FGFR1, FGFR2, FGFR3 or FGFR4 from 100 to 1000 nM, preferably IC₅₀ less than 100 nM, and the best IC₅₀ is less than 10 nM.

The compounds of the present disclosure may be used for treating or preventing FGFR-related tumors, such as non-small cell lung cancer, esophageal cancer, melanoma rhabdomyosarcoma, cell carcinoma, multiple myeloma, breast cancer, ovarian cancer, endometrial cancer, cervical cancer, gastric cancer, colon cancer, bladder cancer, pancreatic cancer, lung cancer, prostate cancer and liver cancers (such as hepatocellular carcinoma), more specifically liver, stomach, and bladder cancers. Thus, in another aspect, the present disclosure provides a method for treating or preventing FGFR-mediated diseases (e.g., tumors). It includes a therapeutically effective amount of the compound of the present disclosure or its prodrug, stable isotope derivatives, polymorphs, solvates of drugs, pharmaceutically acceptable salts, isomers and mixtures thereof, or pharmaceutical compositions comprising the compound.

Another aspect of the present disclosure relates to compounds or their prodrugs, stable isotope derivatives, polymorphs, solvates, pharmaceutically acceptable salts, isomers and mixtures thereof shown in general formula I. It is used for treating or preventing FGFR-mediated diseases, such as tumors or inflammatory diseases, including but not limited to non-small cell lung cancer, esophageal cancer, melanin, rhabdomyosarcoma, wild cell carcinoma, multiple myeloma, breast cancer, ovarian cancer, endometrial cancer, uterine cancer, stomach cancer, diaphragmatic cancer, bladder cancer, pancreatic cancer, lung cancer, prostate cancer.

The present disclosure further relates to a pharmaceutical composition, the pharmaceutical composition comprises the compound of the present disclosure or its prodrug, stable isotope derivatives, medicinal salt isomers and their mixtures thereof and pharmaceutically acceptable carriers, diluents, excipients.

Another aspect of the present disclosure relates to the compound shown in general formula I or a stable isotope derivative thereof, a pharmaceutically acceptable salt, an isomer and a mixture thereof of a prodrug, or the use of the drug composition in the preparation of a drug, wherein the drug used is used for the treatment or prevention of diseases involved in FGFR such as tumors and inflammatory diseases.

According to the present disclosure, the drug may be any pharmaceutical dosage form including but not limited to tablets, capsules, solutions, lyophilized preparations, injections.

### DESCRIPTION AND ABBREVIATIONS OF THE INVENTION

Unless stated otherwise, the terms used in the description and claims of the present disclosure appear below.

The representation “Cx y” with the following meaning and used in this article represents the range of carbon atoms, where x and y are integers. For example, C₃₋₈ cycloalkyl represents a cycloalkyl group with 3-8 carbon atoms, that is, a cycloalkyl group with 3, 4, 5, 6, 7, or 8 carbon atoms. It should also be understood that ‘C₃₋₈’ also includes any sub range therein, such as C₃₋₇, C₃₋₆, C₄₋₇, C₄₋₆, C₅₋₆, etc.

“Alkyl” refers to a straight or branched hydrocarbon group containing 1 to 20 carbon atoms, such as 1 to 18 carbon atoms, 1 to 12 carbon atoms, 1 to 8 carbon atoms, 1 to 6 carbon atoms, or 1 to 4 carbon atoms. Non limiting examples of alkyl groups include methyl, ethyl, n-propyl, isopropyl, n-butyl, isobutyl, tert butyl, sec-butyl, n-amyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl 1,3-dimethylbutyl and 2-ethylbutyl. The alkyl group can be substituted or unsubstituted.

“Alkenyl” refers to a straight or branched hydrocarbon group containing at least one carbon-carbon double bond and typically 2 to 20 carbon atoms, such as 2 to 8 carbon atoms, 2 to 6 carbon atoms, or 2 to 4 carbon atoms. Non limiting examples of alkenyl groups include vinyl, 1-propenyl, 2-propenyl, 1-Butene, 2-butenyl, 3-butenyl, 2-methyl-2-propenyl, 1,4-pentadienyl and 1,4-butadiene. The alkenyl group can be substituted or unsubstituted.

“Alkynyl” refers to a straight or branched hydrocarbon group containing at least one carbon-carbon triple bond and typically 2 to 20 carbon atoms, such as 2 to 8 carbon atoms, 2 to 6 carbon atoms, or 2 to 4 carbon atoms. Non limiting examples of alkynyl groups include acetylene, 1-propargyl, 2-propargyl, 1-butyrgyl, 2-butyrgyl, and 3-butyrgyl groups. The alkynyl group can be substituted or unsubstituted.

“Cycloalkyl” refers to a saturated cyclic hydrocarbon substituent group containing 3 to 14 carbon ring atoms. Cycloalkyl groups can be single carbon rings, typically containing 3 to 7 carbon ring atoms. Non limiting examples of monocyclic cycloalkyl groups include cyclopropyl, cyclobutyl, cyclopentyl, cyclohexyl, and cycloheptyl. The cycloalkyl group can optionally be a fused double or triple ring, such as decahydronaphthyl, and the cycloalkyl group can be substituted or unsubstituted.

“Heterocyclic group”, “heterocyclic alkyl” and “heterocyclic ring” refer to stable 3-18 unit price non aromatic ring, including 2-12 carbon atoms and 1-6 heteroatoms selected from nitrogen, oxygen and sulfur. Unless otherwise specified, heterocyclic groups can be single ring, double ring, triple ring, or quadruple ring systems, which may include fused ring, helical ring, or bridging ring systems. Nitrogen, carbon, or sulfur on heterocyclic groups can be selectively oxidized, nitrogen atoms can be selectively quaternized, and heterocyclic groups can be partially or completely saturated. Heterocyclic groups can be connected to the rest of the molecule through a single bond through carbon or heteroatoms on the ring. Heterocyclic groups containing fused rings can contain one or more aromatic rings or heteroaromatic rings, as long as the atoms on the non aromatic ring are connected with the rest of the molecule. For the purpose of this application, the heterocyclic group preferably consists of a stable 4-11 membered monovalent non aromatic single ring or two rings, comprising 1-3 heteroatoms selected from nitrogen, oxygen, and sulfur, and more preferably a stable 4-8 membered monovalent non aromatic single ring, comprising 1-3 heteroatoms selected from nitrogen, oxygen, and sulfur. Non limiting examples of heterocyclic groups include azacycloheptyl, azacyclobutyl, decahydroisoquinolinyl, dihydrofuranyl, dihydroindolyl, dioxolanyl, 1,1-dioxo-thiomorpholinyl, imidazolinyl, imidazolinyl, isothiazolyl, isoxazolyl, morpholinyl, octahydroindolyl, octahydroisoindolyl, oxazinyl, guazinyl, guaidinyl, 4-guaidinone, pyranyl, pyrazolyl, pyrrolidinyl Quinazinyl, Quinuclidine, tetrahydrofuranyl, Tetrahydropyran, etc.

“Screw heterocyclic group” refers to a multi ring heterocyclic group consisting of 5 to 20 membered rings that share an atom (called a screw atom) between single rings. One or more ring atoms are selected from nitrogen, oxygen, or heteroatoms of S(O) m (where m is an integer 0 to 2), and the remaining ring atoms are carbon. These electronic systems may contain one or more double bonds, but none of the rings have completely conjugated electronic systems, preferably ranging from 6 to 14 elements, and more preferably from 7 to 10 elements. According to the number of shared screw atoms between rings, spiroalkyl groups are divided into single spiroalkyl groups, double spiroalkyl groups, or multiple spiroalkyl groups, preferably single spiroalkyl and double spiroalkyl groups. More preferably, it is a 4-yuan/4-yuan, 4-yuan/5-yuan, 4-yuan/6-yuan, 5-yuan/5-yuan, or 5-yuan/6-yuan single helix ring base. Non limiting embodiments of spirocyclic groups include:

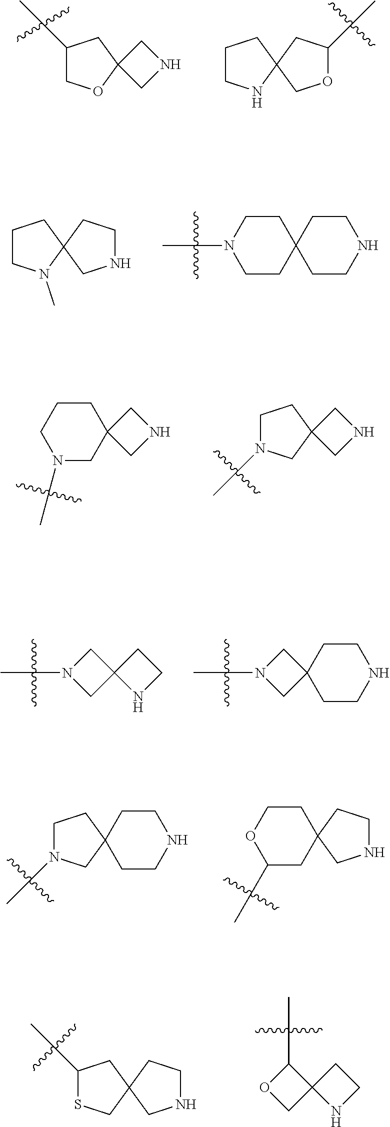

“Fused heterocyclic group” refers to 5 to 20 elements. Each ring in the system shares an adjacent pair of atomic polycyclic heterocyclic group with other rings in the system. One or more rings can contain one or more double bonds, but no ring has a fully conjugated π electronic system. One or more ring atoms are selected from nitrogen, oxygen or S(O) m (where m is an integer 0 to 2), and the remaining ring atoms are carbon. Preferably priced at 6-14 yuan, more preferably priced at 7-10 yuan. According to the number of constituent rings, they can be divided into bicyclic, tricyclic, tetracyclic, or polycyclic fused heterocyclic alkyl groups, preferably bicyclic or tricyclic, and more preferably 5-membered/5-membered or 5-membered/6-membered bicyclic fused heterocyclic groups. Non limiting embodiments of fused heterocyclic groups include:

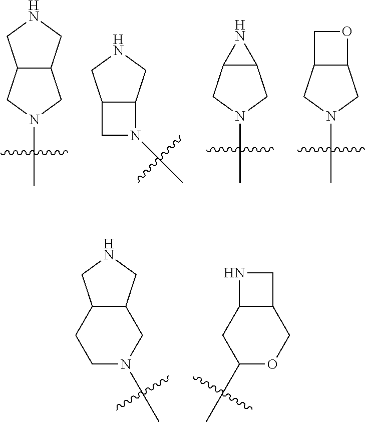

“Aromatic” or “aromatic” refers to aromatic monocyclic or fused polycyclic groups containing 6 to 14 carbon atoms, preferably 6 to 10 elements, such as phenyl and naphthyl, more preferably the phenyl can be condensed onto a heteroaryl, heterocyclic, or cycloalkyl ring, where the ring connected to the parent structure is an aromatic ring, and a non-restrictive example includes:

“Heteroaryl” or “heteroaryl” refers to a 5-16 membered ring system, which contains 1-15 carbon atoms, preferably 1-10 carbon atoms, 1-4 heteroatoms selected from nitrogen, oxygen and sulfur, and at least one aromatic ring. Unless otherwise specified, heteroaryl groups can be single ring, double ring, triple ring, or four ring systems, which may include fused ring or bridging ring systems. As long as the connection point with other parts of the molecule is an aromatic ring atom, the nitrogen, carbon, and sulfur atoms on the heteroaryl ring can be selectively oxidized, and the nitrogen atoms can be selectively quaternized. For the purpose of the present disclosure, the heteroaryl group preferably is a stable 4-11-membered single aromatic ring, which contains 1-3 heteroatoms selected from nitrogen, oxygen and sulfur, and more preferably is a stable 5-8-membered single aromatic ring, which contains 1-3 heteroatoms selected from nitrogen, oxygen and sulfur. Non limiting examples of heteroaryl groups include acridine group, azapyridyl group, Benzimidazole group, benzoindolyl group, benzodioxin group, benzodioxyl group, Benzofuran ketone group, Benzofuran group, benzonaphthofuranyl group, Benzopyran ketone group, Benzopyran group, benzopyrazolyl group, benzothiadiazole group, Benzothiazole group, Benzotriazole group, furanyl group, imidazolyl group, indozolyl group, indolyl group, oxazole base, purinyl, pyrazinyl Pyrazolyl, pyridazinyl, pyridyl, pyrimidinyl, pyrrolyl, Quinazoline, quinolinyl, quininyl, tetrazolyl, thiadiazole, thiazolyl, thiophenyl, triazinyl, triazolyl, etc. In this application, the heteroaryl group is preferably 5-8-membered heteroaryl group, which includes 1-3 heteroatoms selected from nitrogen, oxygen, and sulfur, more preferably pyridine, pyrimidine, and thiazole groups. The heteroaryl group can be substituted or unsubstituted.

“Halogen” refers to fluorine, chlorine, bromine, or iodine.

“Hydroxyl” refers to —OH, “amino” refers to —NH2, “amide” refers to —NHCO—, “cyano” refers to —CN, “nitro” refers to —CN, “isocyano” refers to —NC, and “trifluoromethyl” refers to —CF3.

The terms “heteroatom” or “heteroatom” used alone or as part of other components in this article refer to atoms other than carbon and hydrogen, which are independently selected from oxygen, nitrogen, sulfur, phosphorus, silicon, selenium, and tin, but are not limited to these atoms. In the embodiments where two or more heteroatoms appear, the two or more heteroatoms may be identical to each other, or some or all of the two or more heteroatoms may be different from each other.

The term “thick” or “thick ring” used alone or in combination in this article refers to a circular structure where two or more rings share one or more bonds.

The term “screw” or “spiral ring” used alone or in combination in this article refers to a circular structure where two or more rings share one or more atoms.

“Optional” or “optionally” means that the subsequent described event or environment can but does not necessarily occur, and this description includes the occurrence or absence of the event or environment. For example, ‘optionally substituted heterocyclic groups with alkyl groups’ means that alkyl groups can but do not have to exist, including situations where heterocyclic groups are replaced by alkyl groups and situations where heterocyclic groups are not replaced by alkyl groups.

“Substituted” refers to one or more atoms in a functional group, preferably 5 or 1-3 atoms, independently replaced by a corresponding number of substituents. It goes without saying that substituents are located in their possible chemical positions, and those skilled in the art can determine (through experiments or theory) possible or impossible substitutions without excessive effort. For example, binding free amino or hydroxyl groups to carbon atoms with unsaturated (such as olefins) bonds may be unstable. The substituents include but are not limited to hydroxyl, amino, halogen, cyano, C₁₋₆ alkyl, C₁₋₆ alkoxy, C₂₋₆ alkenyl, C₂₋₆ alkynyl, C₃₋₈ cycloalkyl, etc.

“Pharmaceutical composition” refers to a composition containing one or more of the compounds described herein or their pharmaceutically active acceptable or prodrugs, as well as other components such as pharmaceutically acceptable carriers and excipients. The purpose of the pharmaceutical composition is to promote the administration of drugs to organisms, facilitate the absorption of active ingredients, and thereby exerting biological activity.

“Isomer” refer to a compound with the same molecular formula but different atomic binding properties or orders or different atomic spatial arrangement, and the isomer with different atomic spatial arrangement is called “stereoisomer”. Stereoisomer includes an optical isomer, a geometric isomer and a conformational isomer. The compound of the present disclosure can exist in the form of optical isomer. According to the configuration of substituents around chiral carbon atoms, these optical isomers are in the “R” or “S” configuration. The optical isomers include enantiomer and diastereomer, and methods for preparing and separating optical isomers are known in the art.

The compounds of the present disclosure can also have geometric isomers. Various geometric isomers and mixtures thereof generated by the distribution of substituents around carbon-carbon double bonds, carbon nitrogen double bonds, cycloalkyl or heterocyclic groups are considered in the present disclosure. The substituents around carbon-carbon double bonds or carbon nitrogen bonds are designated as Z or E configurations, and the substituents around cycloalkyl or heterocycles are designated as cis or trans configurations.

The compounds of the present disclosure may also exhibit tautomerism, such as Keto-enol tautomerism.

It should be understood that the present disclosure includes any tautomeric or stereoisomeric form and its mixture, and is not limited to any tautomeric or stereoisomeric form used in the naming of compounds or chemical combinations.

“Isotopes” refer to all isotopes of atoms present in compounds of the present disclosure. Isotopes include those atoms with the same atomic number but different mass numbers. Examples of isotopes suitable for incorporation into compounds of the present invention are hydrogen, carbon, nitrogen, oxygen, phosphorus, fluorine, and chlorine, such as but not limited to ²H, ³H, ¹³C, ¹⁴C, ¹⁵N, ¹⁸O, ³¹P, ³²P, ³⁵S, ¹⁸F, and ³⁶Cl, respectively. The isotopic labeling compounds of the present disclosure can usually be prepared by using appropriate isotopic labeling reagents instead of non isotopic labeling reagents through traditional techniques known to those skilled in the art or by methods similar to those described in the attached embodiments. Such compounds have various potential applications, such as serving as standards and reagents for measuring biological activity. In the case of stable isotopes, such compounds have the potential to advantageously alter biological, pharmacological, or pharmacokinetic properties

“Prodrug” refers to the compound of the present disclosure that can be administered in the form of a prodrug. Prodrugs refer to the derivatives of biologically active compounds invented under physiological conditions in vivo, such as through oxidation, reduction, hydrolysis, etc. (each using enzymes or without enzyme participation). Examples of prodrugs are the following compounds: the amino group in the compound of the present disclosure is acylated, alkylated or phosphorylated, such as icosane acylamino, propylamine amido, pivaloyloxymethyl amino, or the hydroxyl group is acylated, alkylated, phosphorylated or converted to borate, such as acetoxy group, palmitoxy, pivaloyloxy, succinyloxy, fumaroyloxy, propiamoyloxy or the carboxyl group is esterified or amidated, or the mercapto group forms disulfide bridge bonds with carrier molecules that selectively target and/or deliver drugs to the cytosol of cells, such as peptides, these compounds can be prepared by the compounds of the present disclosure according to the well-known methods.

“Medicinal salt” or “pharmaceutically acceptable” refers to a medicinal base or acid, including inorganic base or acid and organic base or acid. In the case where the compound of the present disclosure contains one or more acidic or alkaline groups, the present disclosure also includes their corresponding medicinal salts. Therefore, compounds of the present disclosure containing acidic groups can exist in the form of salts and can be used according to the present disclosure, for example as alkali metal salts, alkali earth metal salts, or as ammonium salts. More exact examples of such salts include sodium salt, potassium salt, calcium salt, magnesium salt or amine or organic amine, such as primary amine, secondary amine, tertiary amine, cyclic amine, etc., such as ammonia, Isopropylamine, Trimethylamine, Diethylamine, triethylamine, Tripropylamine, Ethanolamine, Diethanolamine, Ethanolamine, Dicyclohexylamine, Ethylenediamine, purine, guazine, guaidine, choline, caffeine, and other particularly preferred Organic base are Isopropylamine, Diethylamine, Ethanolamine, Trimethylamine A salt of Dicyclohexylamine, choline, and caffeine. The compounds of the present disclosure containing alkaline groups can exist in salt form and can be used in the form of their addition to inorganic or organic acids according to the present disclosure. Examples of suitable acids include hydrochloric acid, hydrobromic acid, phosphoric acid, sulfuric acid, phosphoric acid, methanesulfonic acid, p-toluenesulfonic acid, naphthalene disulfonic acid, oxalic acid, acetic acid, tartaric acid, lactic acid, salicylic acid, benzoic acid, formic acid, propionic acid, tervaleric acid, malonic acid, succinic acid, pimelic acid, fumaric acid, maleic acid, malic acid, amino sulfonic acid, phenylpropionic acid, gluconic acid, ascorbic acid, isonicotinic acid, citric acid adipic acid and other acids known to those skilled in the art. If the compound of the present disclosure contains both acidic and alkaline groups in the molecule, in addition to the salt form mentioned, the present disclosure also includes inner salts or inner ammonium salts. The salts are obtained by conventional methods known to those skilled in the art, such as by contacting these with organic or mineral acid or bases in solvents or dispersants, or by anion exchange or cation exchange with other salts.

Therefore, when referring to “compound”, “compound of the present disclosure” or “compound of the present disclosure” in this application, all the compound forms, such as its prodrug, stable isotope derivatives, medicinal salts, isomers, racemates, racemic mixture, enantiomer, diastereomers and their mixtures are included.

In this context, the term “tumor” includes benign tumor and malignant tumor (such as cancer).

In this context, the term “cancer” includes various malignant tumors in which Bruton's Tyrosine kinase is involved, including but not limited to non-small cell lung cancer, esophageal cancer, Melanoma, striated muscle pomegranate, cell cancer, Multiple myeloma, breast cancer, ovarian cancer, uterine membrane cancer, cervical cancer, gastric cancer, colon cancer, bladder cancer, pancreatic cancer, lung cancer, breast cancer, prostate cancer and liver cancer (such as hepatocellular carcinoma), more specifically liver cancer Gastric cancer and bladder cancer.

The terms “effective amount”, “therapeutic effective amount”, or “pharmaceutical effective amount” used in this article refer to the amount of at least one medication or compound that is sufficient to alleviate one or more symptoms of the treated disease or condition to some extent after administration. The result may be a reduction and/or remission of signs, symptoms or causes or any other desired change in the biological system. For example, the “effective amount” used for treatment is the amount of composition containing the compounds disclosed in this article required to provide significant symptom relief effects in clinical practice. Techniques such as dose escalation testing can be used for determining the effective amount suitable for any individual case.

The term “polycrystalline form” or “polycrystalline form (phenomenon)” used in the present disclosure refers to the compound of the present disclosure having multiple crystal lattice forms. Some compounds of the present disclosure may have more than one crystal form, and the present disclosure covers all polycrystalline forms or mixtures thereof.

The intermediate compound and its multiple forms of the compound of the present disclosure are also within the scope of the present disclosure.

Crystallization often produces solvate of the compound of the present disclosure. The term “solvate” used herein refers to a combination of one or more compound molecules of the present disclosure and one or more solvent molecules.

The solvent can be water, in this case, the solvate is hydrate. Alternatively, it can be an organic solvent. Therefore, the compounds of the present disclosure can exist as hydrates, including monohydrate, dihydrate, hemihydrate, trihydrate, tetrahydrate, etc., and corresponding solvation forms. The compounds of the present disclosure can be true solvate, but in other cases, the compounds of the present disclosure may only occasionally retain water or a mixture of water and some other solvents. The compounds of the present disclosure can react in a solvent or precipitate or crystallize in a solvent. The solvate of the compound of the present disclosure is also included in the scope of the present disclosure.

The term “acceptable” used in this article in relation to formulations, compositions, or ingredients refers to the absence of sustained harmful effects on the overall health of the treatment subject.

The term “pharmaceutically acceptable” used in this article refers to a substance (such as a carrier or diluent) that does not affect the biological activity or properties of the compound of the present disclosure and is relatively non-toxic, meaning that the substance can be applied to an individual without causing adverse biological reactions or interacting with any component contained in the composition in an adverse manner.

“Pharmaceutically acceptable carriers” include but are not limited to adjuvants, carriers, excipients, additives, deodorants, diluents, preservatives, dyes/colorants, flavor enhancers, surfactants and wetting agents, dispersants, suspensions, stabilizers, and other penetrating agents, solvents, or emulsifiers that have been approved by relevant government administrative departments for use in humans and domesticated animals.

The terms “subject,” “patient,” “object,” or “individual” used in the text refer to individuals suffering from diseases, disorders, or illnesses, including mammals and non mammals. Examples of mammals include but are not limited to any member of the mammalian class: humans, non human primates (such as chimpanzees and other apes and monkeys); livestock, such as cows, horses, sheep, goats, pigs; domestic animals, such as rabbits, dogs, and cats; laboratory animals, including rodents such as rats, mice, and guinea pigs. Examples of non-human mammals include but are not limited to birds and fish. In an embodiment of the method and composition provided in this article, the mammal is a human.

The term “treatment” used in this article refers to the treatment of related diseases and conditions in mammals, especially humans, including
(i) Prevent mammals, especially those who have been previously exposed to a disease or condition but have not been diagnosed with that disease or condition, from developing the corresponding disease or condition;
(ii) Suppress the disease or symptom, i.e. control its development;
(iii) Alleviate the disease or condition, that is, slow down the resolution of the disease or condition;
(iv) Relieve symptoms caused by diseases or symptoms.

The terms “disease” and “disease” used in this article can be substituted for each other or have different meanings, as certain specific diseases or diseases do not yet have known pathogenic factors (so the cause of the disease is not yet clear), so they cannot be recognized as diseases and can only be seen as unwanted conditions or syndromes. The specific symptoms of these syndromes have been confirmed by clinical researchers to some extent.

The terms “take”, “apply”, “administration”, etc. used in this article refer to methods that can deliver a compound or composition to the desired site for biological action, including but not limited to oral route, duodenal route, parenteral injection (including intravenous, subcutaneous, intraperitoneal, intramuscular, arterial injection or infusion), local administration, and rectal administration. In the preferred embodiment, the compounds and compositions discussed in this article are administered orally.

Synthetic Method

The present disclosure provides a method for preparing the compounds. The preparation of the compounds described in General Formula I may be accomplished by exemplary methods and embodiments. These methods and embodiments shall not be considered as limitation of the scope of the present invention in any way. The compounds in question may also be synthesized by a synthesis technique known to those skilled in the art invention, or a combination of methods known in the art and methods described in the present invention. The product obtained is obtained by a separation technique known in the art at each step, including but not limited to extraction, filtration, distillation, crystallization, chromatographic separation, etc. The starting materials and chemical reagents required for synthesis can be routinely synthesized or purchased according to the literature (reaxys).

The alkynyl heterocyclic compound with general formula (IIa) in the present disclosure is prepared by four routes in the following:

Method A, the starting material II-1a is substituted by aromatic nucleophilic reaction to afford II-2a, followed by Sonagashira coupling reaction to afford intermediate II-3a, and then Boc protection group is removed under acidic conditions to afford intermediate II-4a, and finally the compound with structural formula (IIa) is obtained via nucleophilic addition reaction.

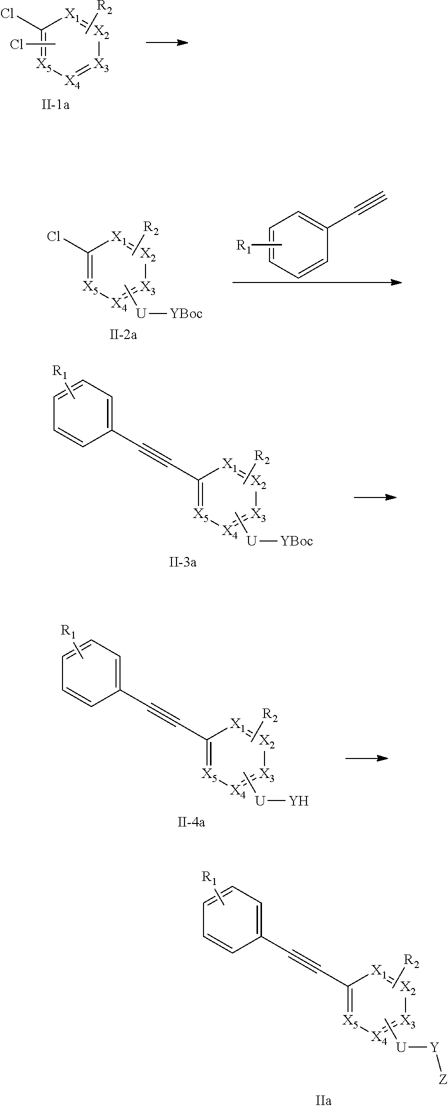

Method B, coupling of starting material II-1a with alkynyl compound affords intermediate II-2b via Suzuki coupling reaction, followed by Sonagashira coupling reaction to afford intermediate II-4a, and then the compound with structural formula (IIa) is obtained by method A.

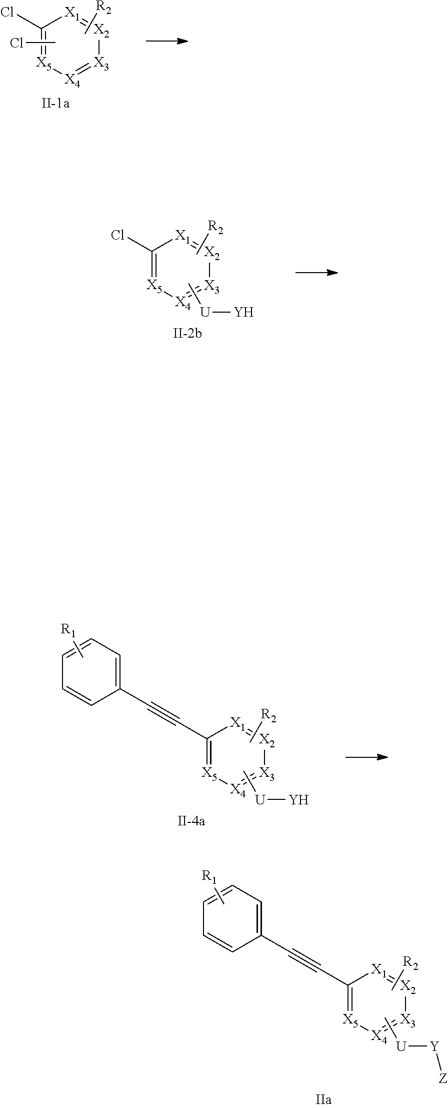

Method C, the starting material II-1a coupled with alkynyl compound affords intermediate II-3c via Sonagashira coupling reaction, followed by aromatic nucleophilic substitution reaction II-3c, and then removing Boc protection group under acidic conditions to afford intermediate II-4a, and finally the compound with structural formula (IIa) is obtained via nucleophilic addition reaction.

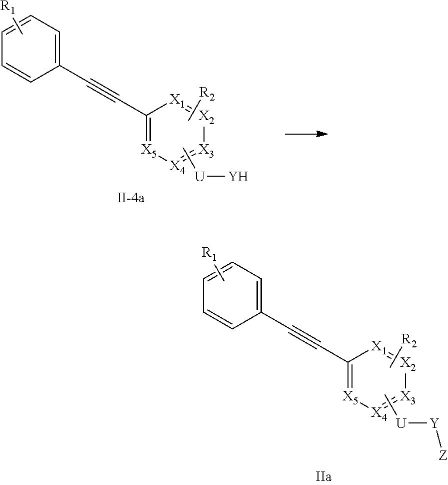

Method D, intermediate II-3c couple with alkynyl compounds via Suzuki coupling reaction to afford intermediate II-4a, and then the compound with structural formula (IIa) is obtained by method A.

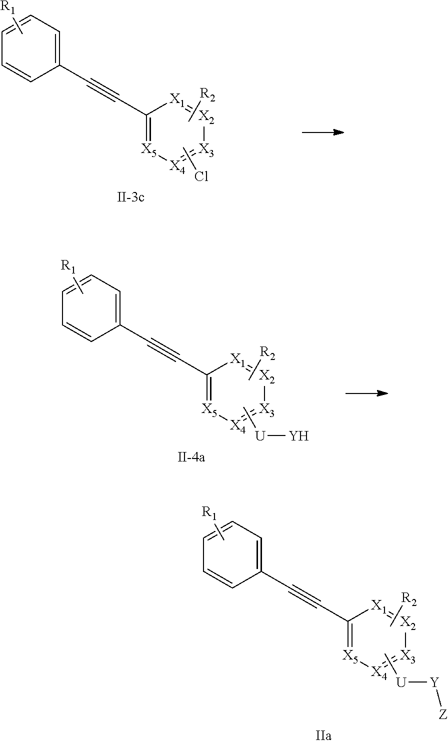

Alkynyl heterocyclic compounds of the general formula (IId) in the present invention is prepared by the following four routes:

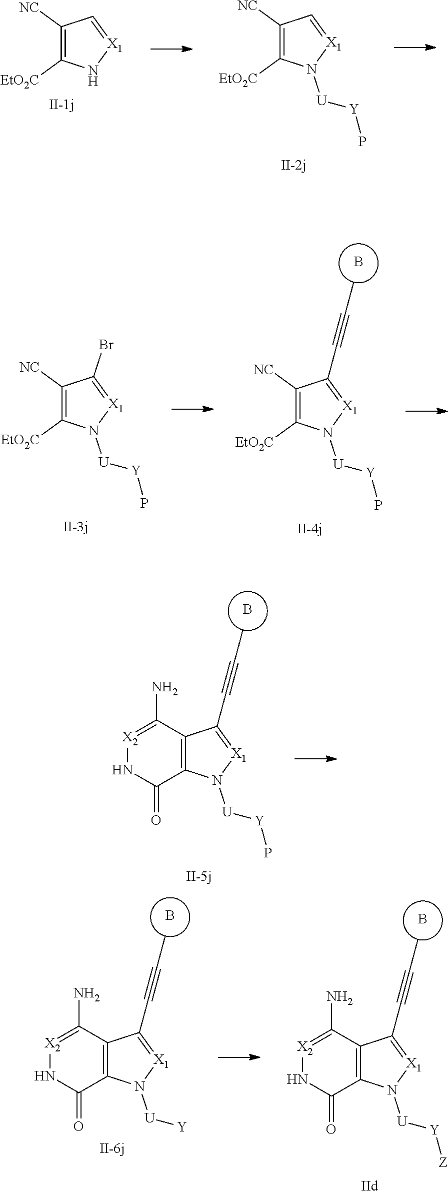

Method I 1. The starting material II-1j and a precursor with hydroxyl groups (HO—U—Y—P) afford II-2j by phototransmission reaction (mitsunobu reaction); 2. II-2j reacts with NBS to afford II-3j via bromination reaction; 3. II-3j and aromatic alkyne are coupled to afford II-4j via sonogashira; 4. II-4j reacts with N₂H₄ to afford II-5j via ring-closing reaction; 5. Amine group deprotection in II-5j to afford II-6j; 6. The amine group in II-6j is derived from the compound of general formula (IId) by a chemical reagent (e.g., allyl chloride, etc.) containing a functional group that reacts with cysteine residues in the kinase ligand binding domain.

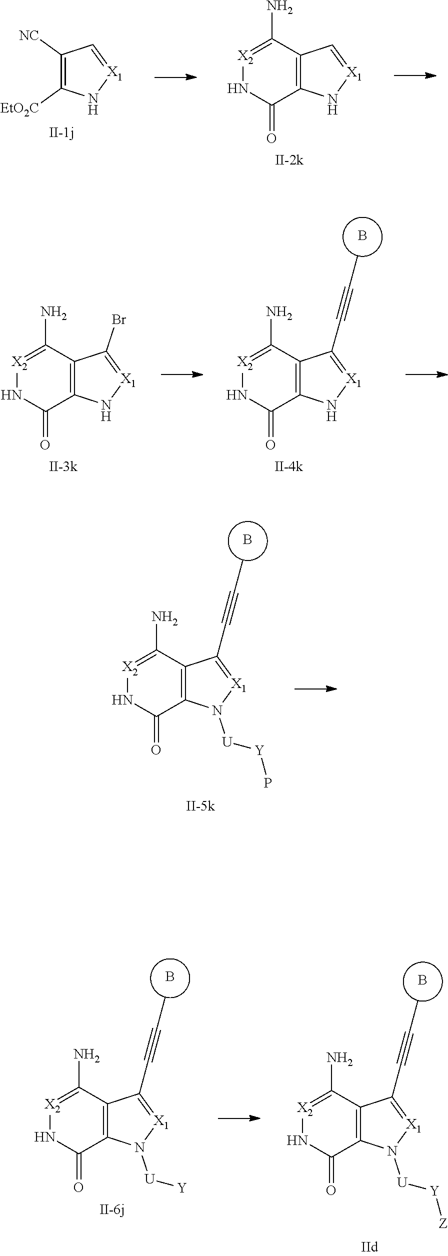

Method J: 1, the starting material II-1j reacts with N₂H₄ to afford II-1k via ring-closing reaction. 2. II-2k reacts with NBS on bromine to afford II-3k via bromination reaction; 3. II-3k and aromatic alkyne are coupled to afford II-4k via Sonogashira Reaction; 4. II-4k and a precursor with hydroxyl groups (HO—U—Y—P) afford II-5k via photoextension reaction (mitsunobu reaction); Then the compound described in general formula (IId) is obtained by the method of the last two steps in Method J.

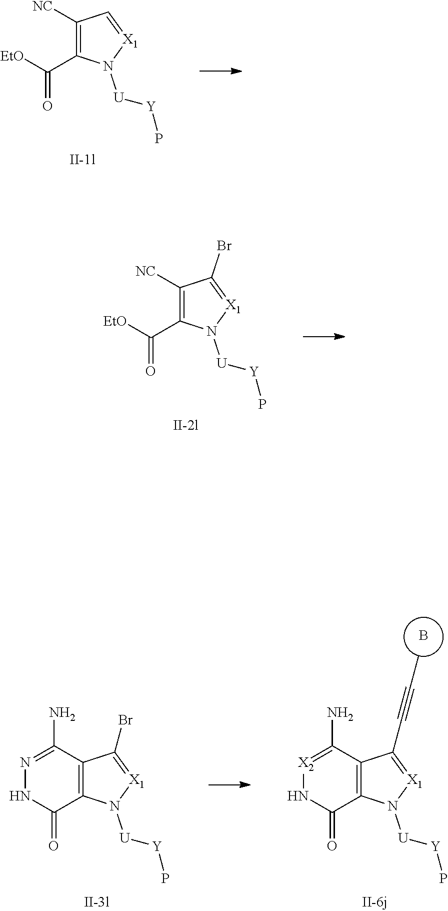

Method K: the starting material II-1l reacts with II-2l to afford intermediate II-2l via bromination reaction. Intermediate II-2l react with N₂H₄ to afford intermediate II-3l via ring-closing reaction. Intermediate II-3l and aromatic alkyne are coupled to afford II-5k via Sonogashira Reaction. Then the compounds of the general formula (IId) are obtained using the methods analogous to J.

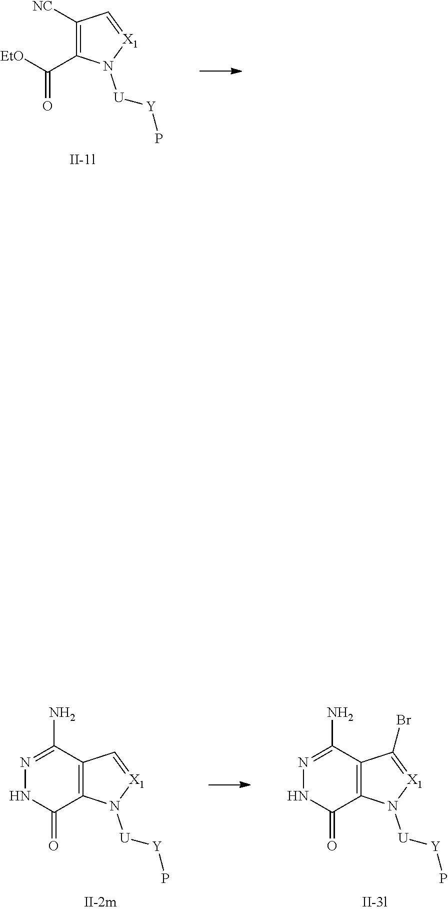

Method L: the starting material II-1l reacts with N₂H₄ to afford intermediate II-2m via ring-closing reaction. Intermediates II-2m react with NBS to afford intermediate II-3lk via bromination reaction. Then the compounds of the general formula (IId) are obtained using the methods analogous to J.

Unless otherwise stated, temperatures are in Celsius. Reagents are purchased from commercial suppliers such as Chemblocks Inc. and ready to be used without further purification unless otherwise noted.

Unless otherwise stated, the following reactions are carried out under N₂ or Ar in anhydrous solvents at room temperature, using dry tubes. Glassware is dried in oven and/or heated to dry.

Unless otherwise stated, column chromatography purification uses 200-300 mesh silica gel from Qingdao Ocean Chemical Plant; The prep.-TLC uses thin layer chromatography silica gel precast plate (HSGF254) produced by Yantai Chemical Industry Research Institute; MS data is collected on Thermo Fisher LCQ Fleet (ESI) liquid chromatography-mass spectrometer.

Nuclear magnetic data (¹H NMR) are collected from Bruker Avance-400 MHz or Varian Oxford-400 Hz NMR, using CDCl₃, CD₃OD, D₂O, DMSO-d₆, etc. as solvent and peak of tetramethylsilane (0.000 ppm) or residual solvent (CDCl₃: 7.26 ppm; CD₃OD: 3.31 ppm; D₂O: 4.79 ppm; DMSO-d₆: 2.50 ppm) as standard. The following abbreviations indicating a variety of peak shapes: s (single peak), d (double peak), t (triple peak), q (quadruple peak), m (multiple peak), br (broad peak), dd (double-double peak), dt (double triple peak). The coupling constant is in Hertz (Hz).

### DETAILED DESCRIPTION OF THE EMBODIMENTS

The present disclosure is further described below through the specific examples.

### Example 1

### Preparation for (S)-1-(3-((4-((3,5-dimethoxyphenyl)ethynyl)pyrimidin-2-yl)amino)pyrrolidin-1-yl)prop-2-en-1-one (Compound 1)

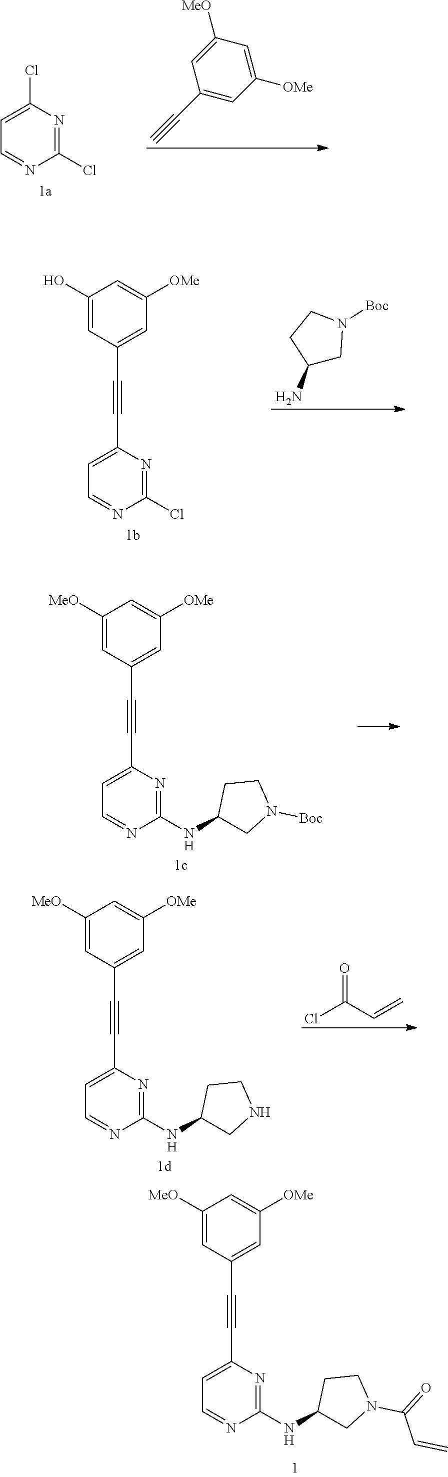

Step 1: synthesis of Compound 1b

The compound 1a (1.49 g, 10.0 mmol), 1-ethynyl-3,5-dimethoxybenzene (1.70 g, 10.5 mmol), Pd(PPh₃)₂Cl₂ (702 mg, 1.0 mmol), CuI (190 mg, 1.0 mmol), Et₃N (5.06 g, 50.0 mmol) and dry DMF (50 mL) were added into a reaction flask under N₂. Vacuumed and filled the reaction flask with N₂ three times. The reaction mixture was stirred at 90° C. overnight, cooled to room temperature, diluted with water and extracted with EtOAc. The obtained organic phase was washed with saturated brine and dried over anhydrous sodium sulfate, depressurized and dried. The residue was purified through column chromatography, so as to obtain the compound 1b (2.14 g, yield 78%). LC/MS(ESI): m/z=275.1[M+H]⁺.

Step 2: Synthesis of Compound 1c

The compound 1b (0.82 g, 3.0 mmol), tert-butyl (S)-3-aminopyrrolidine-1-carboxylate was added (0.67 g, 3.6 mmol), K₂CO₃ (0.83 g, 6.0 mmol) and DMF (12 mL) were added into the reaction flask. The reaction mixture was stirred at 80° C. for 6 h, cooled to room temperature, and diluted with water, extracted with EtOAc. The obtained organic phase was washed with saturated brine and dried over anhydrous sodium sulfate, depressurized and dried. The residue was purified through column chromatography, so as to obtain the compound 1c as yellow solid (1.04 g, yield 82%). LC/MS(ESI): m/z=325.2[M+H]⁺.

Compound 1 (170 mg, yield 45%) obtained by the procedures in the following two steps analogous to example 1 was a yellow solid. ¹H NMR (400 MHz, DMSO-d₆) δ: 8.25 (d, 1H), 6.93 (d, 1H), 6.72 (d, 2H), 6.53-6.48 (m, 2H), 6.21 (dd, 1H), 5.89 (s, 1H), 5.63 (dd, 1H), 4.12-3.98 (m, 1H), 3.81-3.60 (m, 9H), 3.55-3.38 (m, 1H), 2.31-1.89 (m, 2H); LC/MS(ESI): m/z=379.2[M+H]⁺.

### Example 2

### Preparation for (S)-1-(3-((5-bromo-4-((3,5-dimethoxyphenyl)ethynyl)pyrimidin-2-yl)amino)pyrrolidin-1-yl)prop-2-en-1-one (Compound 2)

The compound 2 (156 mg, yield 37%) obtained using a procedure analogous to the procedure described in example 1 was yellow solid. ¹H NMR (400 MHz, DMSO-d₆) δ: 8.34 (s, 1H), 6.72 (d, 2H), 6.51-6.47 (m, 2H), 6.20 (dd, 1H), 5.78 (s, 1H), 5.59 (dd, 1H), 4.09-3.96 (m, 1H), 3.81-3.58 (m, 9H), 3.53-3.34 (m, 1H), 2.30-1.87 (m, 2H); LC/MS(ESI): m/z=457.1[M+H]⁺

### Example 3

### Preparation for (S)-1-(3-((4-((3,5-dimethoxyphenyl)ethynyl)-5-fluoropyrimidin-2-yl)amino)pyrrolidin-1-yl)prop-2-en-1-one (Compound 3)

The compound 3 (149 mg, yield 41%) obtained using a procedure analogous to the procedure described in example 1 was a light yellow solid. ¹H NMR (400 MHz, DMSO-d₆) δ: 8.23 (s, 1H), 6.71 (d, 2H), 6.52-6.46 (m, 2H), 6.19 (dd, 1H), 5.81 (s, 1H), 5.60 (dd, 1H), 4.14-4.03 (m, 1H), 3.83-3.62 (m, 9H), 3.55-3.36 (m, 1H), 2.28-1.85 (m, 2H); LC/MS(ESI): m/z=397.2[M+H]⁺

### Example 4

### Preparation for (S)-1-(3-((4-((3,5-dimethoxyphenyl)ethynyl)-5-(trifluoromethyl)pyrimidin-2-yl)amino)pyrrolidin-1-yl)prop-2-en-1-one (Compound 4)

The compound 4 (124 mg, yield 30%) obtained using a procedure analogous to the procedure described in example 1 was as a light yellow solid. ¹H NMR (400 MHz, DMSO-d₆) δ: 8.48 (s, 1H), 6.73 (d, 2H), 6.52-6.48 (m, 2H), 6.22 (dd, 1H), 5.93 (s, 1H), 5.56 (dd, 1H), 4.14-4.01 (m, 1H), 3.81 (s, 6H), 3.79-3.62 (m, 3H), 3.53-3.32 (m, 1H), 2.24-1.81 (m, 2H); LC/MS(ESI): m/z=447.2[M+H]⁺.

### Example 5

### Preparation for (S)-2-((1-acryloylpyrrolidin-3-yl)amino)-4-((3,5-dimethoxyphenyl)ethynyl)pyrimidine-5-carbonitrile (compound 5)

The compound 5 (126 mg, yield 37%) obtained using a procedure analogous to the procedure described in example 1 was as yellow solid. ¹H NMR (400 MHz, DMSO-d₆) δ: 8.31 (s, 1H), 6.72 (d, 2H), 6.51-6.46 (m, 2H), 6.19 (dd, 1H), 5.81 (s, 1H), 5.60 (dd, 1H), 4.14-4.01 (m, 1H), 3.82-3.60 (m, 9H), 3.56-3.39 (m, 1H), 2.30-1.87 (m, 2H); LC/MS(ESI): m/z=404.2[M+H]⁺.

### Example 6

### Preparation for (S)-2-((1-acryloylpyrrolidin-3-yl)amino)-4-amino-6-((3,5-dimethoxyphenyl)ethynyl)pyrimidine-5-carboxamide (compound 6)

The compound 6 (147 mg, yield 43%) obtained using a procedure analogous to the procedure described in example 1 was as a yellow solid. ¹H NMR (400 MHz, CD₃OD) δ: 6.73 (d, 2H), 6.57 (t, 1H), 6.32 (dd, 1H), 5.76 (dd, 1H), 5.02 (dd, 1H), 4.21-4.09 (m, 1H), 3.97-3.71 (m, 9H), 3.61-3.45 (m, 1H), 2.41-1.92 (m, 2H); LC/MS(ESI): m/z=437.2[M+H]⁺.

### Example 7

### Preparation for 2-((2-acryloyl-2-azaspiro[3.3]heptan-6-yl)amino)-4-amino-6-((3,5-dimethoxyphenyl)ethynyl)pyrimidine-5-carboxamide (compound 7)

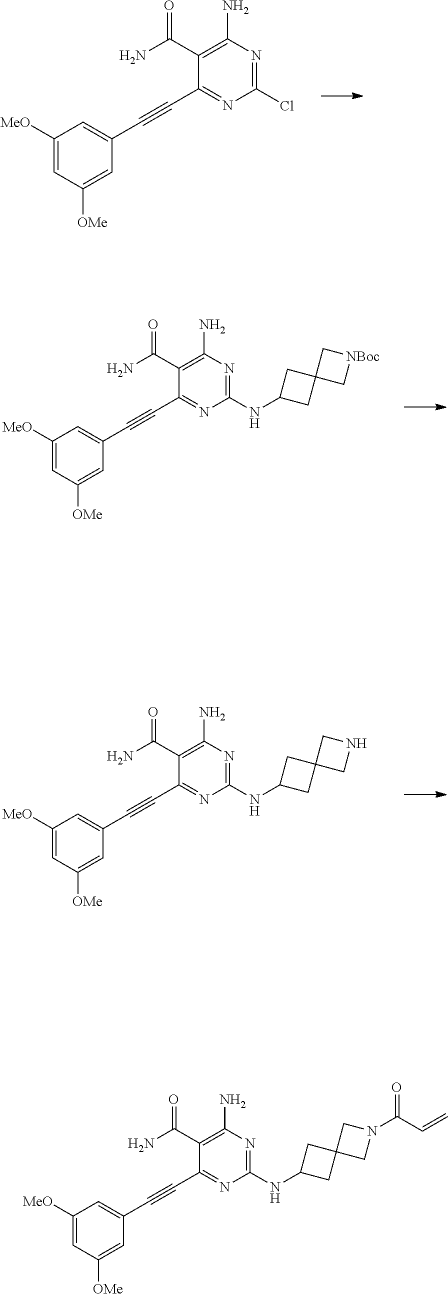 [SMILES: COc1cc(C#Cc2nc(Cl)nc(N)c2C(N)=O)cc(OC)c1 ; COc1cc(C#Cc2nc(NC3CC4(C3)CN(C(=O)OC(C)(C)C)C4)nc(N)c2C(N)=O)cc(OC)c1 ; COc1cc(C#Cc2nc(NC3CC4(CNC4)C3)nc(N)c2C(N)=O)cc(OC)c1 ; C=CC(=O)N1CC2(CC(Nc3nc(N)c(C(N)=O)c(C#Cc4cc(OC)cc(OC)c4)n3)C2)C1]

The compound 7 (127 mg, yield 31%) obtained using a procedure analogous to the procedure described in example 6 was as a yellow solid. ¹H NMR (400 MHz, CD₃OD) δ: 6.75 (d, 2H), 6.60 (t, 1H), 6.43-6.32 (m, 1H), 5.78 (dd, 1H), 5.25-5.19 (m, 1H), 3.80 (s, 6H), 3.67-3.59 (m, 4H), 3.11-3.03 (m, 1H), 2.16-1.92 (m, 4H); LC/MS(ESI): m/z=463.2[M+H]⁺.

### Example 8

### Preparation for 2-((6-acryloyl-6-azaspiro[3.4]octan-2-yl)amino)-4-amino-6-((3,5-dimethoxyphenyl)ethynyl)pyrimidine-5-carboxamide (compound 8)

The compound 8 (134 mg, yield 33%) obtained using a procedure analogous to the procedure described in example 6 was a yellow solid. ¹H NMR (400 MHz, CD₃OD) δ: 6.73 (d, 2H), 6.67-6.58 (m, 1H), 6.32 (dd, 1H), 5.76 (dd, 1H), 5.02-4.93 (m, 1H), 3.80 (s, 6H), 3.35-3.21 (m, 4H), 3.10-3.04 (m, 1H), 2.21-1.92 (m, 4H), 1.62-1.51 (m, 2H); LC/MS(ESI): m/z=477.2[M+H]⁺.

### Example 9

### Preparation for 2-((7-acryloyl-7-azaspiro[3.5]nonan-2-yl)amino)-4-amino-6-((3,5-dimethoxyphenyl)ethynyl)pyrimidine-5-carboxamide (Compound 9)

The compound 9 (141 mg, yield 36%) obtained using a procedure analogous to the procedure described in example 6 was a yellow solid. ¹H NMR (400 MHz, CD₃OD) δ: 6.73 (d, 2H), 6.58 (t, 1H), 6.25 (dd, 1H), 5.78 (dd, 1H), 5.15-5.04 (m, 1H), 3.80 (s, 6H), 3.56-3.32 (m, 4H), 3.11-3.06 (m, 1H), 2.17-1.94 (m, 4H), 1.68-1.52 (m, 4H); LC/MS(ESI): m/z=491.2[M+H]⁺.

### Example 10

### Preparation for 2-((1-acryloylpiperidin-4-yl)amino)-4-amino-6-((3,5-dimethoxyphenyl)ethynyl)pyrimidine-5-carboxamide (compound 10)

The compound 10 (158 mg, yield 45%) obtained using a procedure analogous to the procedure described in example 6 was a yellow solid. ¹H NMR (400 MHz, CD₃OD) δ: 6.73 (d, 2H), 6.57 (t, 1H), 6.24 (dd, 1H), 5.73 (dd, 1H), 4.77 (dd, 1H), 3.80 (s, 6H), 3.65-3.41 (m, 4H), 3.28-3.15 (m, 1H), 2.43-1.91 (m, 4H); LC/MS(ESI): m/z=451.2[M+H]⁺.

### Example 11

### Preparation for (S)-2-((1-acryloylpiperidin-3-yl)amino)-4-amino-6-((3,5-dimethoxyphenyl)ethynyl)pyrimidine-5-carboxamide (compound 11)

The compound 11 (131 mg, yield 31) obtained using a procedure analogous to the procedure described in example 6 was a yellow solid. ¹H NMR (400 MHz, CD₃OD) δ: 6.73 (d, 2H), 6.57 (t, 1H), 6.24 (dd, 1H), 5.73 (dd, 1H), 4.82 (dd, 1H), 3.80-3.34 (m, 10H), 3.18-3.07 (m, 1H), 2.23-1.64 (m, 4H); LC/MS(ESI): m/z=451.2[M+H]⁺.

### Example 12

### (S)-4-amino-2-((1-(but-2-ynoyl)pyrrolidin-3-yl)amino)-6-((3,5-dimethoxyphenyl)ethynyl)pyrimidine-5-carboxamide (compound 12)

The compound 12 (124 mg, yield 28%) obtained using a procedure analogous to the procedure described in example 1 was a yellow solid, using 6d and 2-butynoyl chloride as intermediates. ¹H NMR (400 MHz, CD₃OD) δ: 6.65 (d, 2H), 6.49 (t, 1H), 4.17-4.06 (m, 1H), 3.92-3.70 (m, 9H), 3.58-3.45 (m, 1H), 2.41-2.15 (m, 2H), 1.97 (s, 3H); LC/MS(ESI): m/z=449.2[M+H]⁺.

### Example 13

### Preparation for (S)-1-(3-((5-bromo-4-((3,5-dimethoxyphenyl)ethynyl)pyrimidin-2-yl)amino)pyrrolidin-1-yl)but-2-yn-1-one (compound 13)

The compound 13 (118 mg, yield 22%) obtained using a procedure analogous to the procedure described in example 1 was a yellow solid, using 2d and 2-butynoyl chloride as intermediates. ¹H NMR (400 MHz, DMSO-d₆) δ: 8.35 (s, 1H), 6.73 (d, 2H), 6.50 (t, 1H), 5.84 (s, 1H), 4.11-3.98 (m, 1H), 3.84-3.62 (m, 9H), 3.53-3.38 (m, 1H), 2.31-1.76 (m, 5H); LC/MS(ESI): m/z=469.1[M+H]⁺.

### Example 14

### Preparation for (S)-1-(3-((4-((3,5-dimethoxyphenyl)ethynyl)-5-(trifluoromethyl)pyrimidin-2-yl)amino)pyrrolidin-1-yl)but-2-yn-1-one (compound 14)

The compound 14 (97 mg, yield 18%) obtained using a procedure analogous to the procedure described in example 1 was a yellow solid, using 3d and 2-butynoyl chloride as intermediates. ¹H NMR (400 MHz, DMSO-d₆) δ: 8.51 (s, 1H), 6.73 (d, 2H), 6.48 (t, 1H), 5.95 (s, 1H), 4.21-4.08 (m, 1H), 3.91-3.67 (m, 9H), 3.58-3.42 (m, 1H), 2.37-1.84 (m, 5H); LC/MS(ESI): m/z=459.2[M+H]⁺.

### Example 15

### Preparation for (R)-2-((1-acryloylpyrrolidin-3-yl)amino)-4-amino-6-((3,5-dimethoxyphenyl)ethynyl)pyrimidine-5-carboxamide (compound 15)

The compound 15 (145 mg, yield 41%) obtained using a procedure analogous to the procedure described in example 1 was a yellow solid. ¹H NMR (400 MHz, CD₃OD) δ: 6.73 (d, 2H), 6.57 (t, 1H), 6.32 (dd, 1H), 5.76 (dd, 1H), 5.02 (dd, 1H), 4.21-4.09 (m, 1H), 3.97-3.71 (m, 9H), 3.61-3.45 (m, 1H), 2.41-1.92 (m, 2H); LC/MS(ESI): m/z=437.2[M+H]⁺.

### Example 9

### Preparation for (S)-2-((1-acryloylpyrrolidin-3-yl)amino)-4-((3,5-dimethoxyphenyl)ethynyl)pyrimidine-5-carboxamide (compound 16)

The compound 16 (130 mg, yield 32%) obtained using a procedure analogous to the procedure described in example 1 was a yellow solid. ¹H NMR (400 MHz, DMSO-d₆) δ: 8.32 (s, 1H), 7.42 (s, 2H), 6.73 (d, 2H), 6.51-6.46 (m, 2H), 6.21 (dd, 1H), 5.85 (s, 1H), 5.58 (dd, 1H), 4.15-4.02 (m, 1H), 3.83-3.62 (m, 9H), 3.56-3.39 (m, 1H), 2.30-1.85 (m, 2H); LC/MS(ESI): m/z=422.2[M+H]⁺.

### Example 17

### Preparation for (S)-2-(3-acrylamidopyrrolidin-1-yl)-4-amino-6-((3,5-dimethoxyphenyl)ethynyl)pyrimidine-5-carboxamide (compound 17)

The compound (168 mg, yield 47%) obtained using a procedure analogous to the procedure described in example 1 was a yellow solid. ¹H NMR (400 MHz, CD₃OD) δ: 6.73 (d, 2H), 6.57 (t, 1H), 6.38 (dd, 1H), 6.24 (dd, 1H), 5.62 (dd, 1H), 4.11-3.95 (m, 1H), 3.84-3.68 (m, 9H), 3.58-3.37 (m, 1H), 2.34-1.85 (m, 2H); LC/MS(ESI): m/z=437.2[M+H]⁺.

### Example 18

### Preparation for 2-(1-acryloylpyrrolidin-3-yl)-4-amino-6-((3,5-dimethoxyphenyl)ethynyl)pyrimidine-5-carboxamide (compound 18)

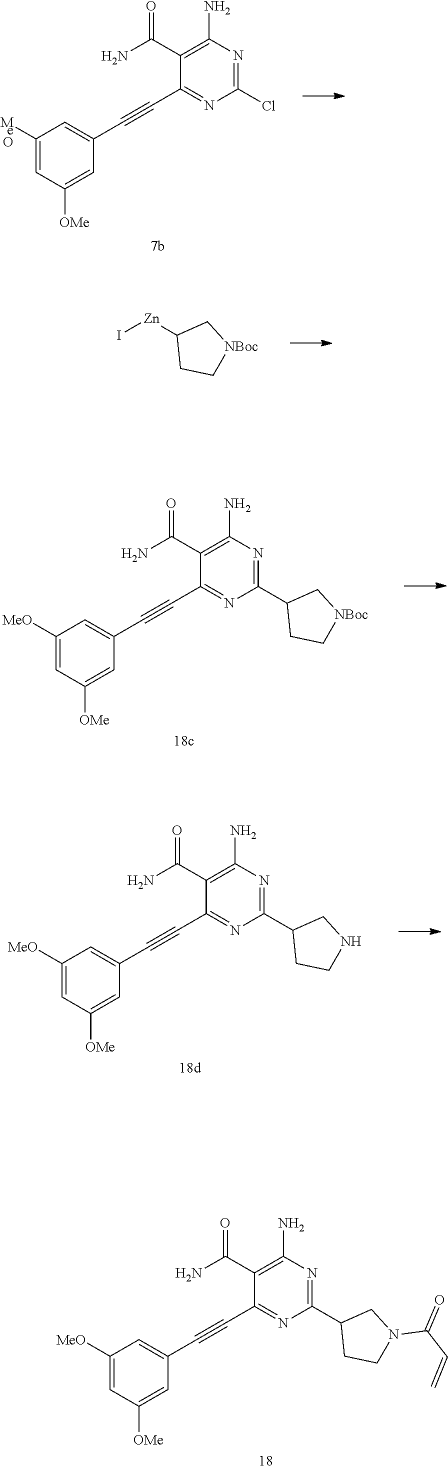

Step 1: Synthesis of Compound 18c

The 4-amino-2-chloro-6-((3,5-dimethoxyphenyl)ethynyl)pyrimidine-5-carboxamide (compound 7b) (3.33 g, 10.0 mmol), Pd(DPPF)₂Cl₂·CH₂Cl₂ (817 mg, 1.0 mmol), CuI (285 mg, 1.5 mmol) and dry DMA (50 mL) were added into the reaction flask. Vacuumed and refilled with N₂ three times, solution of (1-(tert-butoxycarbonyl)pyrrolidin-3-yl)zinc(II) iodide in 2-Methyltetrahydrofuran (15 ml, about 15 mmol) was added. The reaction mixture was stirred at 85° C. for 36 h, cooled to room temperature, diluted with water and extracted with EtOAc. The obtained organic phase was washed with water and saturated brine, dried over anhydrous Na₂SO₄, and then concentrated. The residue was purified through column chromatography to obtain 18c (0.98 g, yield 21%). LC/MS(ESI): m/z=368.2[M+H]⁺.

18 (175 mg, yield 51%) obtained by step 2 and step 3 using the analogous procedure to that in example 1 was a yellow solid. ¹H NMR (400 MHz, CD₃OD) δ: 6.73 (d, 2H), 6.57 (t, 1H), 6.49 (dd, 1H), 6.25 (dd, 1H), 5.52 (dd, 1H), 3.91-3.75 (m, 7H), 3.72-3.58 (m, 1H), 3.52-3.34 (m, 3H), 2.34-1.95 (m, 2H); LC/MS(ESI): m/z=422.2[M+H]⁺.

### Example 19

### Preparation for 2-(1-acryloylpyrrolidin-3-yl)-4-((3,5-dimethoxyphenyl)ethynyl)pyrimidine-5-carboxamide (compound 19)

The compound 19 (155 mg, yield 43%) obtained using a procedure analogous to the procedure described in example 1 was a yellow solid. ¹H NMR (400 MHz, CD₃OD) δ: 6.73 (d, 2H), 6.57 (t, 1H), 6.38 (dd, 1H), 6.24 (dd, 1H), 5.62 (dd, 1H), 4.11-3.95 (m, 1H), 3.84-3.68 (m, 9H), 3.58-3.37 (m, 1H), 2.34-1.85 (m, 2H); LC/MS(ESI): m/z=437.2[M+H]⁺.

### Example 20

### Preparation for (S)-1-(1-acryloylpiperidin-3-yl)-4-amino-3-((3,5-dimethoxyphenyl)ethynyl)-1,6-dihydro-7H-pyrrolo[2,3-d]pyridazin-7-one (compound 20)

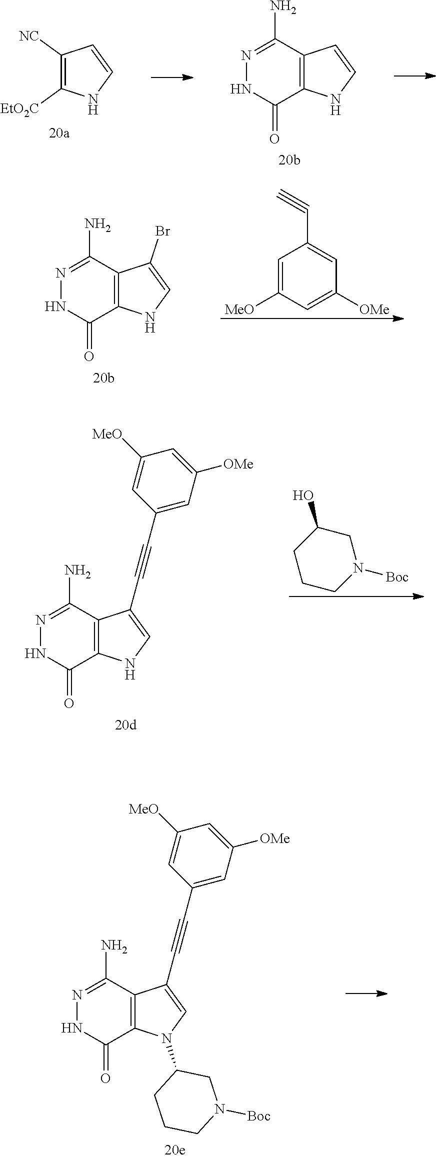

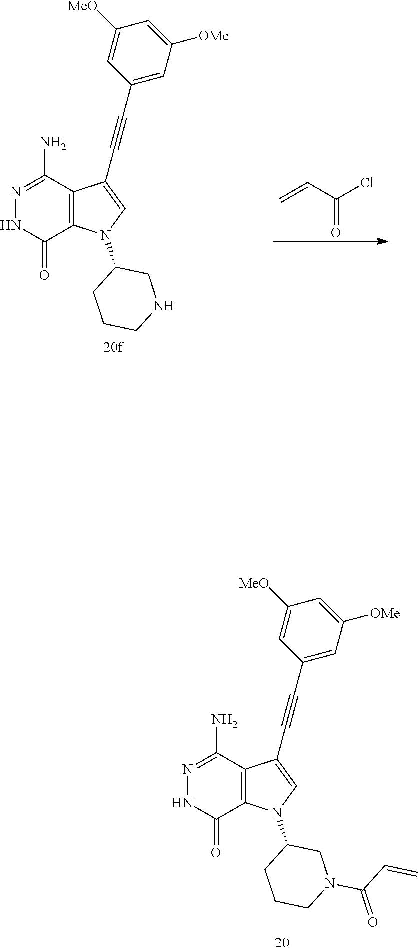

Step 1: Synthesis of Compound 20b

3-cyano-1H-pyrrole-2-carboxylate 20a (1.64 g, 10.0 mmol), hydrazinehydrate (5 mL) and EtOH (50 mL) were added into the reaction flask. The mixture was stirred, heated and refluxed overnight, cooled to room temperature, evaporated; and the residue was purified through column chromatography to obtain the compound 20b (0.64 g, compound 43%) as a white solid. LC/MS(ESI): m/z=151.1[M+H]⁺.

Step 2: Synthesis of Compound 20c

The compound 20b (0.6 g, 4.0 mmol) and DMF (10 mL) and NBS (1.07 g, 6.0 mmol) were added in the reaction flask in batches. The mixture was stirred at 50° C. for 4 hours, cooled to room temperature and poured into 50 mL water, extracted with EtOAc. The obtained organic phase was washed with water and saturated NaCl, dried over anhydrous Na₂SO₄, concentrated; and the residue was purified through column chromatography to obtain the compound 1c (0.63 g, yield 69%) as a white solid. LC/MS(ESI): m/z=229.0[M+H]⁺.

Step 3: Synthesis of Compound 20d

The compound 20c (0.46 g, 2.0 mmol), 3,5-dimethoxybenzene (0.48 g, 3.0 mmol), Pd(PPh₃)₂Cl₂ (140 mg, 0.2 mmol), cuprous iodide (38 mg, 0.2 mmol), triethyl amine (1.01 g, 10.0 mmol), and DMF (15 mL) were added into the reaction flask. The mixture was vacuumed and refilled with N₂ three times. And then the reaction mixture was stirred at 90° C. overnight, cooled to room temperature, diluted with the mixture of EtOAc and water, extracted with EtOAc. The obtained organic phase was washed with water and saturated brine and dried over anhydrous Na₂SO₄, concentrated; and the residue was purified through column chromatography to obtain the compound 1d (0.46 g, yield 74%) as a yellow solid. LC/MS(ESI): m/z=311.1 [M+H]⁺

Step 4: Synthesis of Compound 20e

Tert-butyl (R)-3-hydroxypiperidine-1-carboxylate (241 mg, 1.2 mmol), triphenylphosphine (315 mg, 1.2 mmol), THE (10 mL) were added into the reaction flask, and then DIAD (243 mg, 1.2 mmol) was added. The yellow reaction solution was stirred of 5-10 min. and then added the intermediate 20d (243 mg, 1.2 mmol). The mixture was stirred at room temperature for 12 h, evaporated to obtain the brown oil. The residue oil was purified through column chromatography to obtain the compound 20e (345 mg, yield 70%) as a yellow solid. LC/MS(ESI): m/z=494.2[M+H]⁺.

Step 5: Synthesis of Compound 20f

The intermediate 20e (296 mg, 0.6 mmol), EtOAc (1 mL) and the solution of 4N HCl in dioxane (1 mL) were added into the reaction flask. The mixture was stirred at room temperature for 2 h, neutralized with 1 N NaOH solution and extracted with EtOAc. The obtained organic phase was washed with saturated sodium bicarbonate solution and saturated brine, dried over anhydrous sodium sulfate and concentrated to obtain the compound 20f (227 mg, yield 96%). The crude was used in the next step directly. LC/MS(ESI): m/z=394.2[M+H]⁺.

Step 6: Synthesis of Compound 20

The compound 20f (197 mg, 0.5 mmol), triethylamine (76 mg, 0.75 mmol), DCM (2 mL) were added into the reaction flask, the acryloyl chloride solution in DCM (78 mg, 0.75 mmol, 0.5 mL) was slowly dropped after cooling in ice bath. The reaction mixture was stirred continuously at room temperature for 4 h. The reaction solution was quenched and evaporated. The residue was purified through column chromatography to obtain the compound 20 (96 mg, yield 43%) as a yellow solid. ¹H NMR (400 MHz, DMSO-d₆) δ: 11.52 (s, 1H), 7.53 (s, 1H), 6.82 (d, 2H), 6.53 (t, 1H), 6.42-6.33 (m, 1H), 6.13-6.05 (m, 1H), 5.73-5.63 (m, 1H), 5.13-5.04 (m, 1H), 3.88-3.57 (m, 2H), 3.79 (s, 6H), 3.18-3.08 (m, 2H), 2.23-1.64 (m, 4H); LC/MS(ESI): m/z=448.2 [M+H]⁺.

### Example 21

### Preparation for (S)-4-amino-1-(1-(but-2-ynoyl)piperidin-3-yl)-3-((3,5-dimethoxyphenyl)ethynyl)-1,6-dihydro-7H-pyrrolo[2,3-d]pyridazin-7-one (compound 21)

The compound 21 (80 mg, yield 35) obtained using a procedure analogous to the procedure described in example 20 was a yellow solid. ¹H NMR (400 MHz, DMSO-d₆) δ: 11.53 (s, 1H), 7.54 (s, 1H), 6.82 (d, 2H), 6.54 (t, 1H), 5.13-5.02 (m, 1H), 3.88-3.56 (m, 2H), 3.79 (s, 6H), 3.18-3.09 (m, 2H), 2.23-1.63 (m, 4H), 1.98 (s, 3H); LC/MS(ESI): m/z=460.2[M+H]⁺.

### Example 22

### Preparation for (S)-1-(1-acryloylpyrrolidin-3-yl)-4-amino-3-((3,5-dimethoxyphenyl)ethynyl)-1,6-dihydro-7H-pyrrolo[2,3-d]pyridazin-7-one (compound 22)

The compound 21 (95 mg, yield 44) obtained using a procedure analogous to the procedure described in example 20 was a yellow solid. ¹H NMR (400 MHz, CDCl₃) δ: 9.84-9.90 (d, 1H), 7.37-7.41 (d, 1H), 6.71 (s, 2H), 6.53-6.55 (m, 2H), 6.47-6.48 (m, 1H), 6.35-6.45 (m, 1H), 5.77-5.85 (m, 2H), 3.76-4.23 (m, 11H), 2.59-2.2.68 (m, 1H), 2.37-2.39 (br, 1H); LC/MS(ESI): m/z=434.0[M+H]⁺.

### Example 23

### Preparation for (S)-4-amino-1-(1-(but-2-ynoyl)pyrrolidin-3-yl)-3-((3,5-dimethoxyphenyl)ethynyl)-1,6-dihydro-7H-pyrrolo[2,3-d]pyridazin-7-one (compound 23)

The compound 23 (84 mg, yield 38%) obtained using a procedure (through 2-butynyl chloride reaction) analogous to the procedure described in example 20 was a yellow solid. ¹H NMR (400 MHz, DMSO-d₆) δ: 11.53 (s, 1H), 7.53 (s, 1H), 6.82 (d, 2H), 6.53 (t, 1H), 5.29-5.20 (m, 1H), 4.04-3.95 (m, 2H), 3.79 (s, 6H), 3.63-3.52 (m, 2H), 2.43-2.30 (m, 2H), 1.98 (s, 3H); LC/MS(ESI): m/z=446.2[M+H]⁺.

### Example 24

### Preparation for (S)-1-(1-acryloylpiperidin-3-yl)-4-amino-3-((3,5-dimethoxyphenyl)ethynyl)-1,6-dihydro-7H-pyrazolo[3,4-d]pyridazin-7-one (compound 24)

The compound 24 (102 mg, yield 46%) obtained using a procedure (the raw material was replaced to 3-cyan-1H-pyrazol-2-ethyl formate) analogous to the procedure described in example 20. was a yellow solid. ¹H NMR (400 MHz, DMSO-d6) δ: 11.52 (s, 1H), 6.82 (d, 2H), 6.53 (t, 1H), 6.42-6.33 (m, 1H), 6.13-6.04 (m, 1H), 5.73-5.62 (m, 1H), 5.13-5.02 (m, 1H), 3.88-3.56 (m, 2H), 3.79 (s, 6H), 3.18-3.07 (m, 2H), 2.23-1.64 (m, 4H); LC/MS(ESI): m/z=449.2 [M+H]⁺.

### Example 25

### Preparation for (S)-4-amino-1-(1-(but-2-ynoyl)piperidin-3-yl)-3-((3,5-dimethoxyphenyl)ethynyl)-1,6-dihydro-7H-pyrazolo[3,4-d]pyridazin-7-one (Compound 25)

The compound 25 (84 mg, yield 37%) obtained using a procedure (through the intermediate and 2-butynyl chloride reaction) analogous to the procedure described in example 20 was a yellow solid. ¹H NMR (400 MHz, DMSO-d₆) δ: 11.53 (s, 1H), 6.82 (d, 2H), 6.54 (t, 1H), 5.13-5.02 (m, 1H), 3.88-3.56 (m, 2H), 3.79 (s, 6H), 3.18-3.08 (m, 2H), 2.23-1.63 (m, 4H), 1.98 (s, 3H); LC/MS(ESI): m/z=461.2[M+H]⁺.

### Example 26

### Preparation for (S)-1-(1-acryloylpyrrolidin-3-yl)-4-amino-3-((3,5-dimethoxyphenyl)ethynyl)-1,6-dihydro-7H-pyrazolo[3,4-d]pyridazin-7-one (Compound 26)

The compound 26 (90 mg, yield 42%) obtained using a procedure (the raw material was replaced to 3-cyan-1H-pyrazol-2-ethyl formate, and the intermediate was replaced to (R)-1-t-butyloxycarboryl-pyrrolidinol) analogous to the procedure described in example 20 was a white-off solid. ¹H NMR (400 MHz, DMSO-d₆) δ: 11.52 (s, 1H), 6.82 (d, 2H), 6.53 (t, 1H), 6.42-6.33 (m, 1H), 6.13-6.04 (m, 1H), 5.73-5.62 (m, 1H), 5.31-5.22 (m, 1H), 4.05-3.97 (m, 2H), 3.78 (s, 6H), 3.63-3.52 (m, 2H), 2.43-2.32 (m, 2H); LC/MS(ESI): m/z=435.2[M+H]⁺.

### Example 27

### Preparation for (S)-4-amino-1-(1-(but-2-ynoyl)pyrrolidin-3-yl)-3-((3,5-dimethoxyphenyl)ethynyl)-1,6-dihydro-7H-pyrazolo[3,4-d]pyridazin-7-one (Compound 27)

The compound 27 (88 mg, yield 40%) obtained using a procedure (through the intermediate and 2-butynyl chloride reaction) analogous to the procedure described in example 20 was an off-white solid. ¹H NMR (400 MHz, DMSO-d₆) δ: 11.53 (s, 1H), 6.82 (d, 2H), 6.53 (t, 1H), 5.30-5.21 (m, 1H), 4.05-3.96 (m, 2H), 3.79 (s, 6H), 3.63-3.52 (m, 2H), 2.43-2.31 (m, 2H), 1.98 (s, 3H); LC/MS(ESI): m/z=447.2[M+H]⁺.

### Example 28

### Preparation for (S)-1-(1-acryloylpyrrolidin-3-yl)-4-amino-3-((2,6-difluoro-3,5-dimethoxyphenyl)ethynyl)-1,6-dihydro-7H-pyrazolo[3,4-d]pyridazin-7-one (Compound 28)

The compound 28 (95 mg, yield 44%) obtained using a procedure (the intermediate was replaced to 3,5-dimethoxy-2,6-difluorophenylene) analogous to the procedure described in example 20 was an off-white solid. ¹H NMR (400 MHz, DMSO) δ: 11.58 (s, 1H), 8.06-8.11 (d, 1H), 7.14-7.10 (s, 1H), 6.69-6.55 (m, 1H), 6.22-6.15 (m, 1H), 6.18-5.97 (m, 1H), 5.99-5.68 (m, 1H), 5.60 (br, 2H), 4.14-4.13 (m, 1H), 3.90 (s, 6H), 3.54-3.99 (m, 3H), 2.47-2.39 (m, 2H); LC/MS(ESI): m/z=470.0[M+H]⁺.

### Example 29

### Preparation for (S)-4-amino-1-(1-(but-2-ynoyl)pyrrolidin-3-yl)-3-((2,6-difluoro-3,5-dimethoxyphenyl)ethynyl)-1,6-dihydro-7H-pyrazolo[3,4-d]pyridazin-7-one (Compound 29)

The compound 29 (112 mg, yield 52%) obtained using a procedure (the intermediate was replaced to 3,5-dimethoxy-2,6-difluorophenylene) analogous to the procedure described in example 20 was a yellow solid. LC/MS(ESI): m/z=482.0[M+H]⁺.

### Example 30

### Preparation for (S)-1-(1-acryloylpyrrolidin-3-yl)-4-amino-3-((2,6-dichloro-3,5-dimethoxyphenyl)ethynyl)-1,6-dihydro-7H-pyrazolo[3,4-d]pyridazin-7-one (Compound 30)

The compound 30 (95 mg, yield 41%) obtained using a procedure (the intermediate was replaced to 3,5-dimethoxy-2,6-difluorophenylene) analogous to the procedure described in example 20 was a white-off solid. LC/MS(ESI): m/z=503.0[M+H]⁺.

### Example 31

### Preparation for (S)-4-amino-1-(1-(but-2-ynoyl)pyrrolidin-3-yl)-3-((2,6-dichloro-3,5-dimethoxyphenyl)ethynyl)-1,6-dihydro-7H-pyrazolo[3,4-d]pyridazin-7-one (Compound 31)

The compound 31 (134 mg, yield 57%) obtained using a procedure (the intermediate was replaced to 3,5-dimethoxy-2,6-difluorophenylene) analogous to the procedure described in example 20. was a yellow solid. LC/MS(ESI): m/z=514.0[M+H]⁺.

### Example 32

### Preparation for (S)-4-amino-1-(1-(but-2-ynoyl)pyrrolidin-3-yl)-3-((2,6-difluoro-3,5-dimethoxyphenyl)ethynyl)-1,6-dihydro-7H-pyrazolo[3,4-d]pyridazin-7-one (Compound 32)

The compound 31 (73 mg, yield 34%) obtained using a procedure (the intermediate was replaced to 3,5-dimethoxy-2,6-difluorophenylene) analogous to the procedure described in example 19 was a yellow solid. LC/MS(ESI): m/z=471.0[M+H]⁺.

### Example 33

### Preparation for (S)-4-amino-1-(1-(but-2-ynoyl)pyrrolidin-3-yl)-3-((2,6-difluoro-3,5-dimethoxyphenyl)ethynyl)-1,6-dihydro-7H-pyrazolo[3,4-d]pyridazin-7-one (Compound 33)

The compound 33 (129 mg, yield 58%) obtained using a procedure (the intermediate was replaced to 3,5-dimethoxy-2,6-difluorophenylene) analogous to the procedure described in example 19 was a yellow solid. LC/MS(ESI): m/z=483.0[M+H]⁺.

### Example 34

### Preparation for (S)-4-amino-1-(1-(but-2-ynoyl)pyrrolidin-3-yl)-3-((2,6-difluoro-3,5-dimethoxyphenyl)ethynyl)-1,6-dihydro-7H-pyrazolo[3,4-d]pyridazin-7-one (Compound 34)

The compound 34 (88 mg, yield 38%) obtained using a procedure (the intermediate was replaced to 3,5-dimethoxy-2,6-difluorophenylene) analogous to the procedure described in example 20 was a yellow solid. LC/MS(ESI): m/z=503.0[M+H]⁺.

### Example 35

### Preparation for (S)-4-amino-1-(1-(but-2-ynoyl)pyrrolidin-3-yl)-3-((2,6-dichloro-3,5-dimethoxyphenyl)ethynyl)-1,6-dihydro-7H-pyrazolo[3,4-d]pyridazin-7-one (Compound 35)

The compound 35 (134 mg, yield 57%) obtained using a procedure (the intermediate was replaced to 3,5-dimethoxy-2,6-difluorophenylene) analogous to the procedure described in example 20 was a yellow solid. LC/MS(ESI): m/z=515.0[M+H]⁺.

### Example 36

### Preparation for (S)-1-(1-acryloylpyrrolidin-3-yl)-4-amino-3-(7-methoxy-5-methylbenzo[b]thiophen-2-yl)-1,6-dihydro-7H-pyrazolo[3,4-d]pyridazin-7-one (Compound 36)

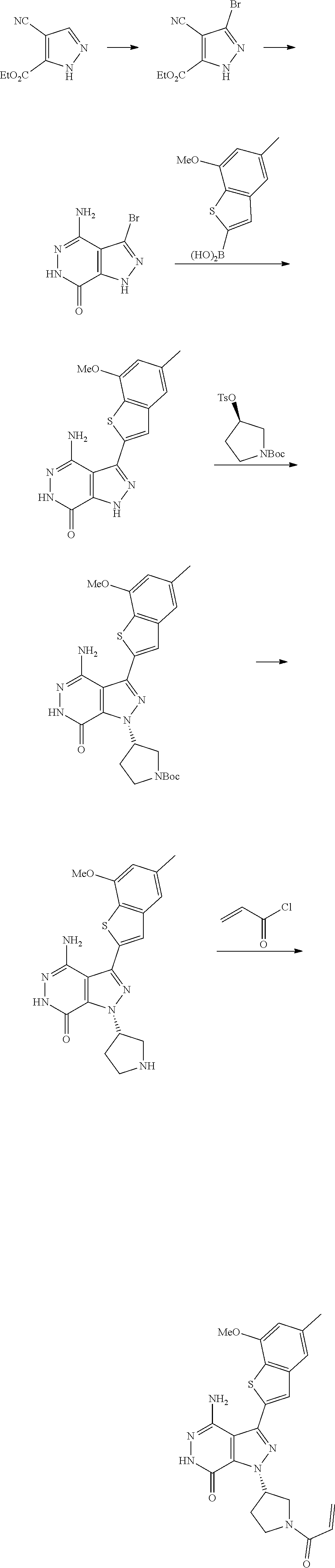

The compound 4-cyano-1H-pyrazole-5-carboxylate (10 g, 4.0 mmol) and DMF (100 mL) were added to the reaction flask, NBS (1.07 g, 6.0 mmol) was added in batches. The mixture was stirred at 50° C. for 4 h, cooled to room temperature, the reaction mixture was poured into 50 mL of water, extracted with EtOAc. The organic phase was washed with water and NaCl saturated aqueous, dried over anhydrous Na₂SO₄, and concentrated; and the residue was purified through column chromatography to obtain the ethyl 3-bromo-4-cyano-1H-pyrazole-5-carboxylate as white solid (0.63 g, yield 69%). LC/MS(ESI): m/z=245.0[M+H]⁺.

The ethyl 3-bromo-4-cyano-1H-pyrazole-5-carboxylate (1.64 g, 10.0 mmol)m hydrozinehydrate (5 mL) and ethanol (50 mL) were added into the reaction flask. The mixture was stirred, heated refluxed for overnight, cooled to room temperature, and the solvent was concentrated. The residue was purified through column chromatography to obtain 4-amino-3-bromo-1,6-dihydro-7H-pyrazolo[3,4-d]pyridazin-7-one (0.64 g, yield 43%) as a white solid. LC/MS(ESI): m/z=230 [M+H]⁺

4-amino-3-bromo-1,6-dihydro-7H-pyrazolo[3,4-d]pyridazin-7-one (0.46 g, 2.0 mmol), (7-methoxy-5-methylbenzo[b]thiophen-2-yl)boronic acid (0.48 g, 3.0 mmol), Pd(PPh₃)₂Cl₂ (140 mg, 0.2 mmol), CuI (38 mg, 0.2 mmol), Et₃N (1.01 g, 10.0 mmol) and DMF (15 mL) were added into the reaction flask. The mixture was vacuumed and refilled with N₂ three times. The mixture was stirred at 90° C. overnight, cooled to room temperature. The reaction solution was diluted with EtOAc and water, extracted with EtOAc, the obtained organic phase was washed with water and saturated brine again, dried over anhydrous Na2SO4 and concentrated. The residue was purified through column chromatograph to obtain 4-amino-3-(7-methoxy-5-methylbenzo[b]thiophen-2-yl)-1,6-dihydro-7H-pyrazolo[3,4-d]pyridazin-7-one (0.46 g, yield 74%) as a yellow solid. LC/MS(ESI): m/z=328[M+H]⁺.

Tert-butyl (R)-3-(tosyloxy)pyrrolidine-1-carboxylate (241 mg, 1.2 mmol), PPh₃ (315 mg, 1.2 mmol) and 10 mL of THE were added into the reaction flask, and then DIAD (243 mg, 1.2 mmol) was added. The yellow solution was stirred for 5-10 minutes and then 4-amino-3-(7-methoxy-5-methylbenzo[b]thiophen-2-yl)-1,6-dihydro-7H-pyrazolo[3,4-d]pyridazin-7-one (310 mg, 1.0 mmol) was added. The mixture was stirred at room temperature for 12 h, evaporated to obtain the brown oil. The residue was purified through column chromatograph to obtain tert-butyl (S)-3-(4-amino-3-(7-methoxy-5-methylbenzo[b]thiophen-2-yl)-7-oxo-6,7-dihydro-1H-pyrazolo[3,4-d]pyridazin-1-yl)pyrrolidine-1-carboxylate (345 mg, yield 70) as a yellow solid. LC/MS(ESI): m/z=497[M+H]⁺.

The intermediate (S)-3-(4-amino-3-(7-methoxy-5-methylbenzo[b]thiophen-2-yl)-7-oxo-6,7-dihydro-1H-pyrazolo[3,4-d]pyridazin-1-yl)pyrrolidine-1-carboxylate (296 mg, 0.6 mmol), 1 mL of EtOAc and 1 mL of dioxane of 4N HCl were added into the reaction flask. The mixture was stirred at room temperature for 2 h. The reaction solution was neutralized with 1N sodium solution, extracted with EtOAc. The obtained organic phase was washed with saturated sodium bicarbonate and saturated brine, dried over anhydrous Na₂SO₄. The organic phase was concentrated to obtain the compound (S)-4-amino-3-(7-methoxy-5-methylbenzo[b]thiophen-2-yl)-1-(pyrrolidin-3-yl)-1,6-dihydro-7H-pyrazolo[3,4-d]pyridazin-7-one (227 mg, yield 96%). The crude was used in the next step directly. LC/MS(ESI): m/z=397.2[M+H]⁺.

The compound (S)-4-amino-3-(7-methoxy-5-methylbenzo[b]thiophen-2-yl)-1-(pyrrolidin-3-yl)-1,6-dihydro-7H-pyrazolo[3,4-d]pyridazin-7-one (197 mg, 0.5 mmol), triethylamine (76 mg, 0.75 mmol) and 2 mL of DCM were added into the reaction flask. 0.5m L of DCM of the acryloyl chloride (78 mg, 0.75 mmol) was slowly added after ice water bath. The reaction mixture was continuously stirred for 4 h after complete of addition. The reaction mixture was quenched with methanol and concentrated. The residue was purified through column chromatograph to obtain compound 36 (96 mg, yield 43%) as a yellow solid. ¹H NMR (400 MHz, DMSO-d₆) δ: 11.52 (s, 1H), 7.53 (s, 1H), 6.82 (d, 2H), 6.53 (t, 1H), 6.42-6.33 (m, 1H), 6.13-6.05 (m, 1H), 5.73-5.63 (m, 1H), 5.13-5.04 (m, 1H), 3.88-3.57 (m, 2H), 3.79 (s, 6H), 3.18-3.08 (m, 2H), 2.23-1.64 (m, 4H); LC/MS(ESI): m/z=451.2 [M+H]⁺.

### Example 37

### Preparation for (S)-4-amino-1-(1-(but-2-ynoyl)pyrrolidin-3-yl)-3-(7-methoxy-5-methylbenzo[b]thiophen-2-yl)-1,6-dihydro-7H-pyrazolo[3,4-d]pyridazin-7-one (Compound 37)

The compound (S)-4-amino-3-(7-methoxy-5-methylbenzo[b]thiophen-2-yl)-1-(pyrrolidin-3-yl)-1,6-dihydro-7H-pyrazolo[3,4-d]pyridazin-7-one (197 mg, 0.5 mmol), triethylamine (76 mg, 0.75 mmol) and 2 mL of DCM were added into the reaction flask. 0.5m L of DCM of the butynoyl chloride (78 mg, 0.75 mmol) was slowly added after an ice water bath. The reaction mixture was continuously stirred for 4 h after complete of addition. The reaction mixture was quenched with methanol and concentrated. The residue was purified through column chromatograph to obtain compound 37 (86 mg, yield 37%) as a yellow solid. LC/MS(ESI): m/z=463.2 [M+H]⁺.

### Example 38

### Preparation for (S)-1-(1-acryloylpyrrolidin-3-yl)-4-amino-3-(7-methoxy-5-methylbenzo[b]thiophen-2-yl)-1,6-dihydro-7H-pyrrolo[2,3-d]pyridazin-7-one (Compound 38)

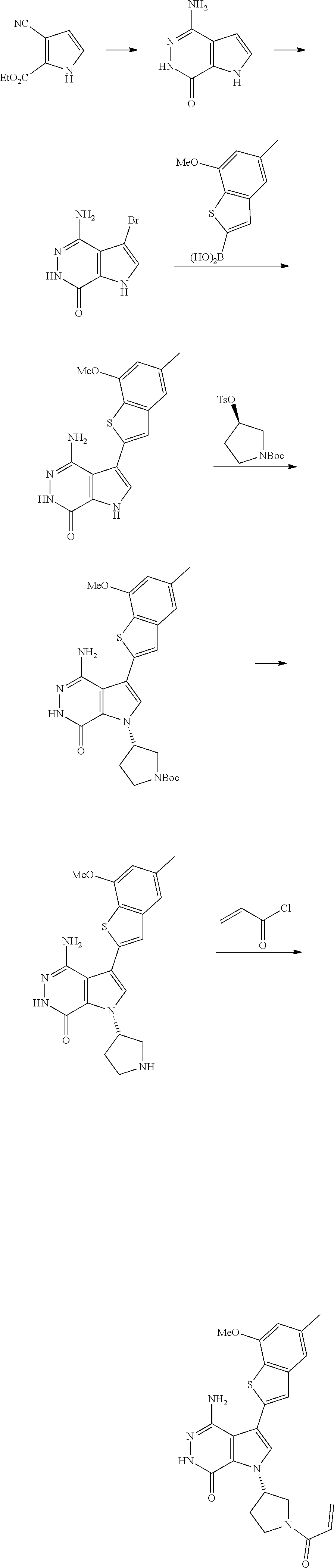

The ethyl 3-cyano-1H-pyrrole-2-carboxylate (1.64 g, 10.0 mmol), hydrozinhydrate (5 mL) and ethanol (50 mL) were added into the reaction flask. The reaction mixture was stirred, heated and refluxed for overnight, cooled to room temperature and concentrated. The residue was purified through column chromatograph to obtain compound 4-amino-1,6-dihydro-7H-pyrrolo[2,3-d]pyridazin-7-one (0.64 g, yield 43%) as a white solid. LC/MS(ESI): m/z=151.1[M+H]⁺.

The compound 4-amino-1,6-dihydro-7H-pyrrolo[2,3-d]pyridazin-7-one (0.60 g, 4.0 mmol) and DMF (10 mL) were added into the reaction flask, NBS (1.07 g, 6.0 mmol) was added in batches. The mixture was stirred and reacted at 50° C. for 4 h, cooled to room temperature, the reaction mixture was poured into 50 mL of water and extracted with EtOAc. The organic phase was washed with water and saturated NaCl, dried over anhydrous Na₂SO₄ and concentrated; and the residue was purified through column chromatograph to obtain 4-amino-3-bromo-1,6-dihydro-7H-pyrrolo[2,3-d]pyridazin-7-one (0.59 g, yield 65%) as a white solid. LC/MS(ESI): m/z=229.0[M+H]⁺.

4-amino-3-bromo-1,6-dihydro-7H-pyrrolo[2,3-d]pyridazin-7-one (0.46 g, 2.0 mmol), (7-methoxy-5-methylbenzo[b]thiophen-2-yl)boronic acid (0.48 g, 3.0 mmol), Pd(PPh₃)₂Cl₂ (140 mg, 0.2 mmol), CuI (38 mg, 0.2 mmol), Et₃N (1.01 g, 10.0 mmol) and DMF (15 mL) were added into the reaction flask. The mixture was refilled with N₂ three times. The mixture was stirred and reacted at 90° C. for overnight, cooled to room temperature. The reaction solution was diluted with EtOAc and water, extracted with EtOAc, the obtained organic phase was washed with water and saturated brine again, dried over anhydrous Na₂SO₄ and concentrated. The residue was purified through column chromatograph to obtain 4-amino-3-(7-methoxy-5-methylbenzo[b]thiophen-2-yl)-1,6-dihydro-7H-pyrrolo[2,3-d]pyridazin-7-one (0.44 g, yield 71%) as a yellow solid. LC/MS(ESI): m/z=327.1[M+H]⁺.

Tert-butyl (R)-3-(tosyloxy)pyrrolidine-1-carboxylate (241 mg, 1.2 mmol), PPh₃ (315 mg, 1.2 mmol) in 10 mL of THF were added in the reaction flask, and then DIAD (243 mg, 1.2 mmol) was added. The yellow solution was stirred for 5-10 minutes and then 4-amino-3-(7-methoxy-5-methylbenzo[b]thiophen-2-yl)-1,6-dihydro-7H-pyrrolo[2,3-d]pyridazin-7-one (310 mg, 1.0 mmol) was added. The mixture was stirred and reacted at room temperature for 12 h, evaporated from solvent under reduced pressure to obtain brown oil. The residue was purified through column chromatograph to obtain (S)-3-(4-amino-3-(7-methoxy-5-methylbenzo[b]thiophen-2-yl)-7-oxo-6,7-dihydro-1H-pyrazolo[3,4-d]pyridazin-1-yl)pyrrolidine-1-carboxylate (320 mg, yield 65%) as a yellow solid. LC/MS(ESI): m/z=496.2[M+H]⁺.

The intermediate (S)-3-(4-amino-3-(7-methoxy-5-methylbenzo[b]thiophen-2-yl)-7-oxo-6,7-dihydro-1H-pyrazolo[3,4-d]pyridazin-1-yl)pyrrolidine-1-carboxylate (296 mg, 0.6 mmol), 1 mL of EtOAc and 1 mL of 1,4-dioxane solution of 4N HCl were added into the reaction flask. The mixture was stirred and reacted at room temperature for 2 h. The reaction solution was neutralized with 1N sodium solution, extracted with EtOAc. The obtained organic phase was washed with saturated sodium bicarbonate and saturated brine, dried over anhydrous Na₂SO₄. The organic phase was concentrated under reduced pressure to obtain (S)-4-amino-3-(7-methoxy-5-methylbenzo[b]thiophen-2-yl)-1-(pyrrolidin-3-yl)-1,6-dihydro-7H-pyrrolo[2,3-d]pyridazin-7-one (220 mg, yield 93%). The crude was used in the next step directly. LC/MS(ESI): m/z=396.1[M+H]⁺.

The compound (S)-4-amino-3-(7-methoxy-5-methylbenzo[b]thiophen-2-yl)-1-(pyrrolidin-3-yl)-1,6-dihydro-7H-pyrrolo[2,3-d]pyridazin-7-one (197 mg, 0.5 mmol), triethylamine (76 mg, 0.75 mmol) and 2 mL of DCM were added into the reaction flask. DCM (0.5 mL) of acryloyl chloride (78 mg, 0.75 mmol) was slowly dropped after an ice water bath. The mixture was continuously stirred for 4 h after complete of addition. The reaction solution was quenched with methanol and concentrated. The residue was purified through column chromatograph to obtain compound 38 (92 mg, yield 41%) as a yellow solid. ¹H NMR (400 MHz, MeOD) δ: 7.74 (d, 1H), 7.37 (s, 1H), 7.27 (s, 1H), 6.78 (s, 1H), 6.72-6.63 (m, 1H), 6.41-6.30 (m, 1H), 6.24-6.16 (m, 1H), 5.82-5.77 (m, 1H), 4.26-3.67 (m, 4H), 3.99 (s, 3H), 2.67-2.51 (m, 2H), 2.51 (s, 3H); LC/MS(ESI): m/z=450.2 [M+H]⁺.

### Example 39

### Preparation for (S)-4-amino-1-(1-(but-2-ynoyl)pyrrolidin-3-yl)-3-(naphthalen-2-yl)-1,6-dihydro-7H-pyrrolo[2,3-d]pyridazin-7-one (Compound 39)

The compound (S)-4-amino-3-(7-methoxy-5-methylbenzo[b]thiophen-2-yl)-1-(pyrrolidin-3-yl)-1,6-dihydro-7H-pyrrolo[2,3-d]pyridazin-7-one (197 mg, 0.5 mmol), triethylamine (76 mg, 0.75 mmol) and 2 mL of DCM were added into the reaction flask. 0.5 mL of DCM of 2-butynol chloride (78 mg, 0.75 mmol) was slowly dropped after an ice water bath. The mixture was continuously stirred for 4 h after completion of addition. The reaction solution was quenched with methanol and concentrated. The residue was purified through column chromatograph to obtain compound 39 (81 mg, yield 35%) as a yellow solid. ¹H NMR (400 MHz, DMSO-d₆) δ: 11.52 (br, 1H), 7.83 (d, 1H), 7.80 (s, 1H), 7.46 (s, 1H), 7.31 (s, 1H), 6.86 (s, 1H), 6.15-6.08 (m, 1H), 5.09 (br, 2H), 4.21-4.17 (m, 1H), 3.99 (s, 3H), 3.67-3.52 (m, 2H), 2.50 (s, 3H), 2.02-2.07 (d, 3H); LC/MS(ESI): m/z=462.2 [M+H]⁺.

### Example 40

### Preparation for (S)-1-(1-acryloylpyrrolidin-3-yl)-4-amino-3-(1-methyl-1H-indol-2-yl)-1,6-dihydro-7H-pyrrolo[2,3-d]pyridazin-7-one (Compound 40)

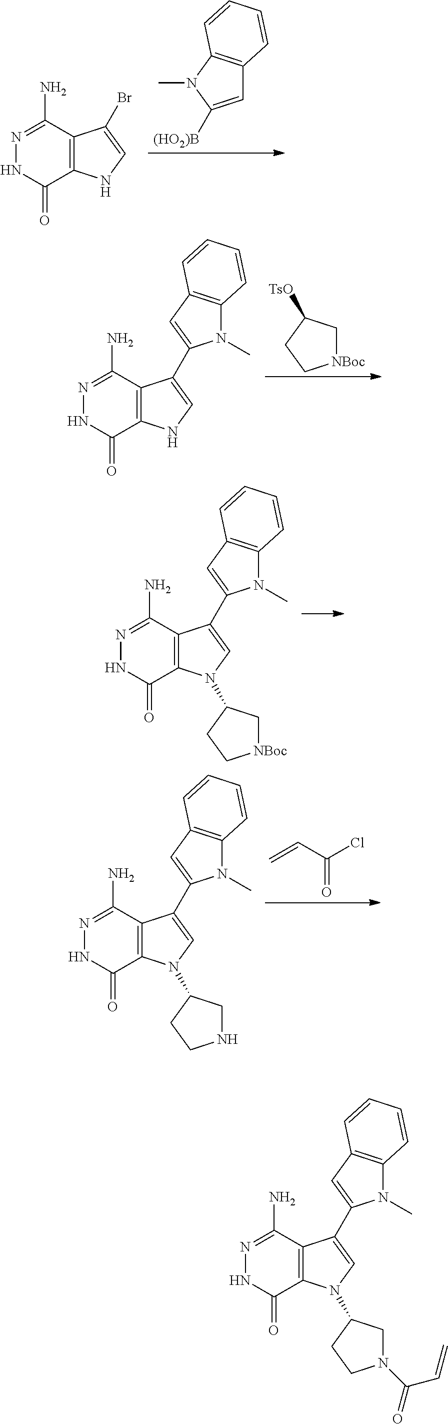

The compound 40 (86 mg, yield 43%) obtained using a procedure (the intermediate was replaced to 3-br-4-amino-1,6-dihydro-7H-pyrrole [3,4-d] pyridazine-7-ketone and 1-methylindole-2-boric acid) analogous to the procedure described in example 29 was a yellow solid. LC/MS(ESI): m/z=403.2[M+H]⁺.

### Example 41

### Preparation for (S)-1-(1-acryloylpyrrolidin-3-yl)-4-amino-3-(benzofuran-2-yl)-1,6-dihydro-7H-pyrrolo[2,3-d]pyridazin-7-one (Compound 41)

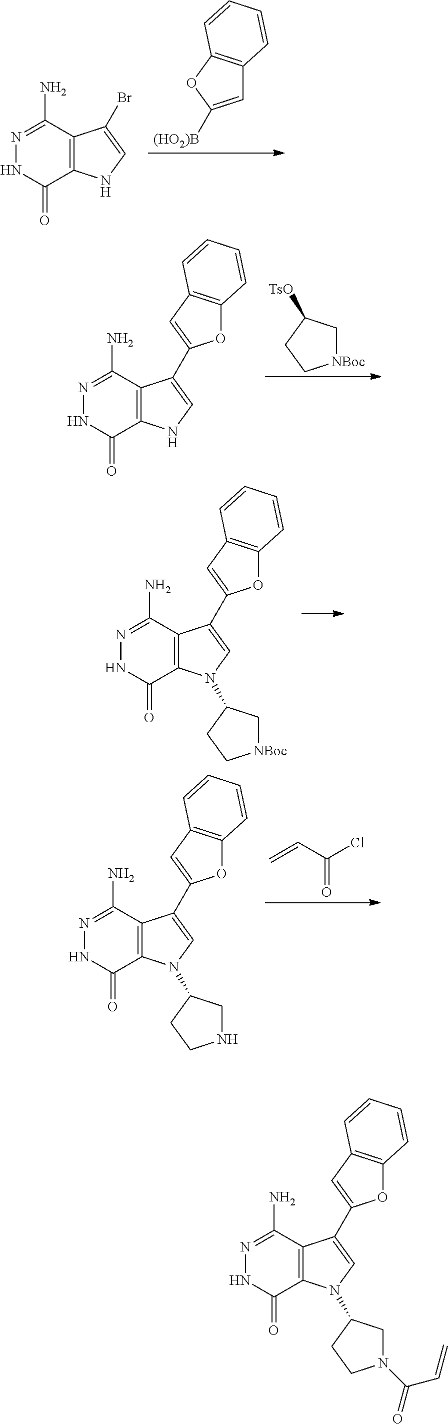

The compound 41 (76 mg, yield 39%) obtained using a procedure (the intermediate was replaced to 3-br-4-amino-1,6-dihydro-7H-pyrrole [3,4-d] pyridazine-7-ketone and benzofuran-2-boric acid) analogous to the procedure described in example 29 was a yellow solid. LC/MS(ESI): m/z=390.2[M+H]⁺.

### Example 42

### Preparation for (S)-1-(1-acryloylpyrrolidin-3-yl)-4-amino-3-(1-methyl-1H-indol-3-yl)-1,6-dihydro-7H-pyrrolo[2,3-d]pyridazin-7-one (Compound 42)

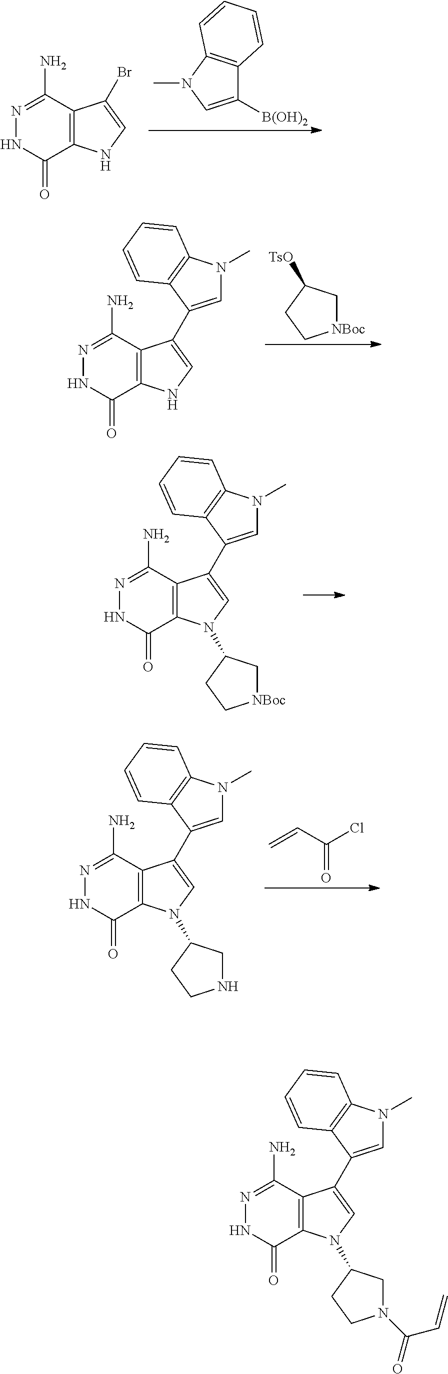

The compound 42 (93 mg, yield 46%) obtained using a procedure (the intermediate was replaced to 3-br-4-amino-1,6-dihydro-7H-pyrrole [3,4-d] pyridazine-7-ketone and benzofuran-2-boric acid) analogous to the procedure described in example 28 was a yellow solid. LC/MS(ESI): m/z=403.2[M+H]⁺.

### Example 43

### Preparation for (S)-1-(1-acryloylpyrrolidin-3-yl)-4-amino-3-(naphthalen-2-yl)-1,6-dihydro-7H-pyrrolo[2,3-d]pyridazin-7-one (Compound 43)

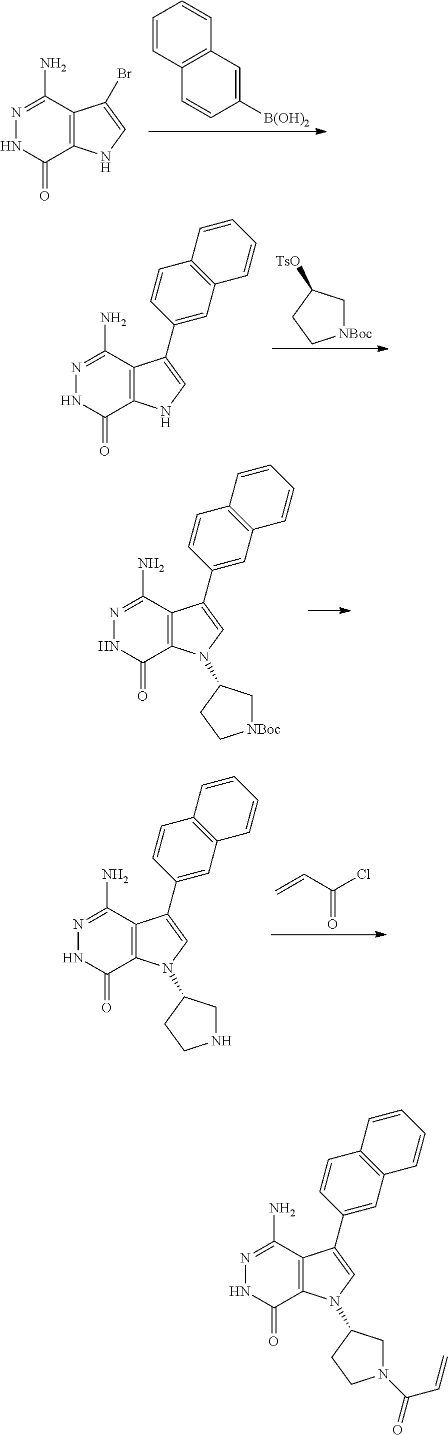

The compound 43 (80 mg, yield 40%) obtained using a procedure (the intermediate was replaced to 3-br-4-amino-1,6-dihydro-7H-pyrrole [3,4-d] pyridazine-7-ketone and 2-naphthylboronic acid) analogous to the procedure described in example 29 was a yellow solid. LC/MS(ESI): m/z=400.2[M+H]⁺.

### Example 44

### Preparation for (S)-1-(3-(8-amino-1-(7-methoxy-5-methylbenzo[b]thiophen-2-yl)imidazo[1,5-a]pyrazin-3-yl)pyrrolidin-1-yl)prop-2-en-1-one (Compound 44)

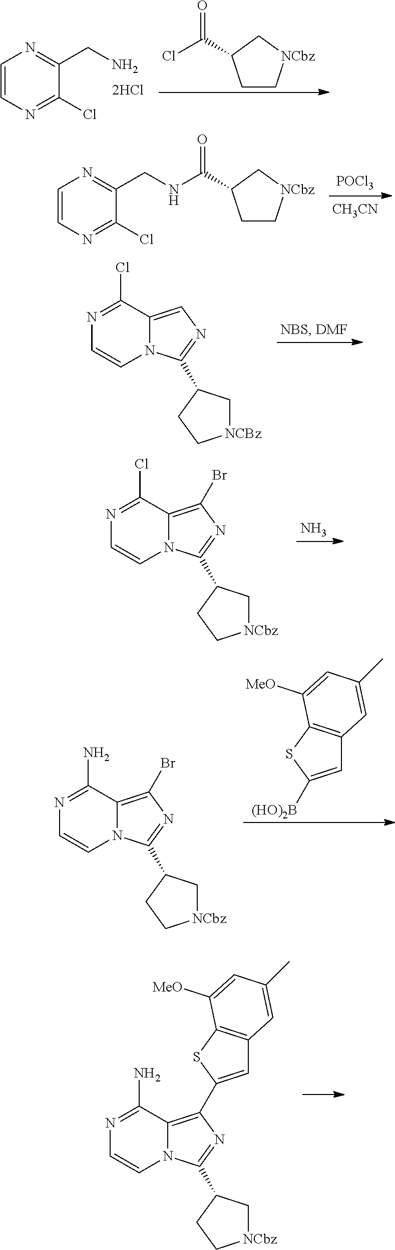

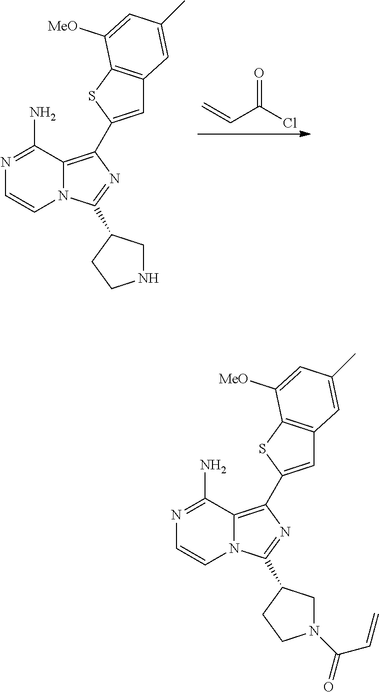

(3-chloropyrazin-2-yl)methanamine dihydrochloride (2.16 g, 10 mmol) and 50 mL of DCM were added into the reaction flask, 10 mL of DCM of N-Cbz-pyrrolidine-3-formyl chloride (3.21 g, 12 mmol) was dropped at 0° C. And then the mixture was heated to room temperature and stirred for 0.5 h. The mixture was quenched with saturated sodium bicarbonate solution To separate the organic phase. The water phase was extracted with DCM, the organic phase was combined and washed with saturated brine. The combined organic phase was dried over anhydrous Na₂SO₄, filtered to remove the drying agent, and concentrated under the reduced pressure to obtain the crude product. (S)-3-(((3-chloropyrazin-2-yl)methyl)carbamoyl)pyrrolidine-1-carboxylate (2.74 g, yield 73%) is obtained through fast column purification. LC/MS(ESI): m/z=375.1[M+H]⁺.

Benzyl (S)-3-(((3-chloropyrazin-2-yl)methyl)carbamoyl)pyrrolidine-1-carboxylate (1.87 g, 5 mmol) and 25 mL of acetonitrile were added into the reaction flask. 4 mL of POCl₃ and DMF were dropped at the room temperature. The mixture was heated to 80° C. and reacted for 2 h under nitrogen protection, cooled to room temperature and the solvent was concentrated under reduced pressure. The residue was poured into ice-water and reacted with DCM. The obtained organic phase was washed with saturated sodium bicarbonate and saturated NaCl aqueous solution, dried over anhydrous Na₂SO₄, and the organic phase was evaporated under reduced pressure. The residue was purified through column chromatograph to obtain benzyl (S)-3-(8-chloroimidazo[1,5-a]pyrazin-3-yl)pyrrolidine-1-carboxylate (0.73 g, yield 41%). LC/MS(ESI): m/z=357.1[M+H]⁺.

(S)-3-(8-chloroimidazo[1,5-a]pyrazin-3-yl)pyrrolidine-1-carboxylate (0.71 g, 2.0 mmol) in DMF (6 mL) and 6 mL of NBS were added into the reaction flask, NBS (0.54 g, 4.0 mmol) was added in batches. The mixture was stirred at the room temperature and reacted for 3 h. The reaction mixture was poured into 50 mL of water, extracted with EtOAc. The organic phase was washed with water and saturated brine, dried over anhydrous Na₂SO₄, evaporated under reduced pressure. The residue was purified through column chromatograph to obtain benzyl (S)-3-(1-bromo-8-chloroimidazo[1,5-a]pyrazin-3-yl)pyrrolidine-1-carboxylate (0.65 g, yield 75%) as a white solid. LC/MS(ESI): m/z=435.0[M+H]⁺.

(S)-3-(1-bromo-8-chloroimidazo[1,5-a]pyrazin-3-yl)pyrrolidine-1-carboxylate (0.65 g, 1.5 mmol), 10 mL of isopropanol and ammonia (30%, 2 mL) were added into the reaction flask. The mixture was stirred, reflexed and reacted for 5 h, and cooled to the room temperature, the reaction solution was diluted with water, extracted with EtOAc. The organic phase was washed with saturated brine and dried over anhydrous Na₂SO₄. The residue was purified through column chromatograph to obtain benzyl (S)-3-(8-amino-1-bromoimidazo[1,5-a]pyrazin-3-yl)pyrrolidine-1-carboxylate (0.54 g, yield 87%) as white solid. LC/MS(ESI): m/z=416.3[M+H]⁺.

Benzyl (S)-3-(8-amino-1-bromoimidazo[1,5-a]pyrazin-3-yl)pyrrolidine-1-carboxylate (0.50 g, 1.2 mmol), (7-methoxy-5-methylbenzo[b]thiophen-2-yl)boronic acid (0.29 g, 1.8 mmol), PdP(Ph₃)₂Cl₂ (140 mg, 0.2 mmol), CuI (140 mg, 0.2 mmol), Et₃N (0.5 g, 5.0 mmol) and 10 mL of DMF were added into the reaction flask. The mixture was refilled with N₂ three times. The mixture was stirred at 90° C. and reacted for overnight, cooled to the room temperature. The reaction solution was diluted with EtOAc and water, extracted with EtOAc. The obtained organic phase was washed with saturated brine again, dried over anhydrous Na₂SO₄ and concentrated under reduced pressure. The residue was purified through column chromatograph to obtain benzyl (S)-3-(8-amino-1-(7-methoxy-5-methylbenzo[b]thiophen-2-yl)imidazo[1,5-a]pyrazin-3-yl)pyrrolidine-1-carboxylate (0.52 g, yield 85%) as a yellow solid. LC/MS(ESI): m/z=514.2[M+H]⁺.

Benzyl (S)-3-(8-amino-1-(7-methoxy-5-methylbenzo[b]thiophen-2-yl)imidazo[1,5-a]pyrazin-3-yl)pyrrolidine-1-carboxylate (0.52 g, 1.0 mmol) and 4 mL of concentrate HCl were added into the reaction flask. The mixture reacted for 24 h at the room temperature. The reaction solution was poured into water. The reaction mixture was neutralized with 1N NaOH solution to be alkalescent, extracted with DCM. The obtained organic phase was washed with saturated sodium bicarbonate, dried over anhydrous Na2SO4, concentrated under reduced pressure to obtain (S)-1-(7-methoxy-5-methylbenzo[b]thiophen-2-yl)-3-(pyrrolidin-3-yl)imidazo[1,5-a]pyrazin-8-amine. (0.38 g, yield 85%) C/MS(ESI): m/z=451.2[M+H]⁺.

(S)-1-(7-methoxy-5-methylbenzo[b]thiophen-2-yl)-3-(pyrrolidin-3-yl)imidazo[1,5-a]pyrazin-8-amine (225 mg, 0.5 mmol), triethylamine (76 mg, 0.75 mmol), and in 2 mL of DCM were added into the reaction flask. 0.5 mL of DCM was slowly dropped after an ice water bath, the mixture was continuously stirred for 4 h after completion addition. The reaction solution was quenched with methanol and concentrated under reduced pressure. The residue was purified through column chromatograph to obtain compound 44 (70 mg, yield 32%) as yellow solid. ¹H NMR (400 MHz, DMSO-d₆) δ: 7.64 (s, 1H), 7.26-7.14 (m, 3H), 6.79 (s, 1H), 6.42-6.33 (m, 1H), 6.13-6.04 (m, 1H), 5.91-5.78 (br s, 2H), 5.73-5.62 (m, 1H), 4.18-3.95 (m, 3H), 3.91 (s, 3H), 3.64-3.53 (m, 2H), 2.49-2.30 (m, 5H); LC/MS(ESI): m/z=434.2 [M+H]⁺.

### Example 45

### Preparation for (S)-1-(3-(8-amino-1-(7-methoxy-5-methylbenzo[b]thiophen-2-yl)imidazo[1,5-a]pyrazin-3-yl)pyrrolidin-1-yl)but-2-yn-1-one (Compound 45)

(S)-4-amino-3-(7-methoxy-5-methylbenzo[b]thiophen-2-yl)-1-(pyrrolidin-3-yl)-1,6-dihydro-7H-pyrrolo[2,3-d]pyridazin-7-one (190 mg, 0.5 mmol), triethylamine (76 mg, 0.75 mmol) and 2 mL of DCM were added into the reaction flask. 0.5 mL of DCM of butyl-2-alkynyl chloride (78 mg, 0.75 mmol) was slowly dropped after an ice water bath, the mixture was continuously stirred for 4 h after completion addition. The reaction solution was quenched with methanol and concentrated under reduced pressure. The residue was purified through column chromatograph to obtain compound 45 (82 mg, yield 37%) as yellow solid. LC/MS(ESI): m/z=446.2 [M+H]⁺.

### Example 46

### Preparation for (S)-1-(3-(4-amino-5-(7-methoxy-5-methylbenzo[b]thiophen-2-yl)imidazo[5,1-f][1,2,4]triazin-7-yl)pyrrolidin-1-yl)prop-2-en-1-one (Compound 46)

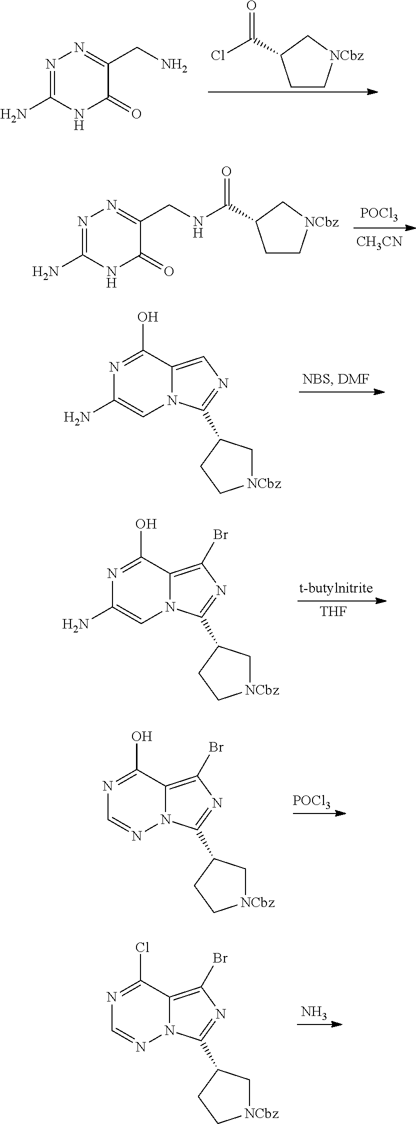

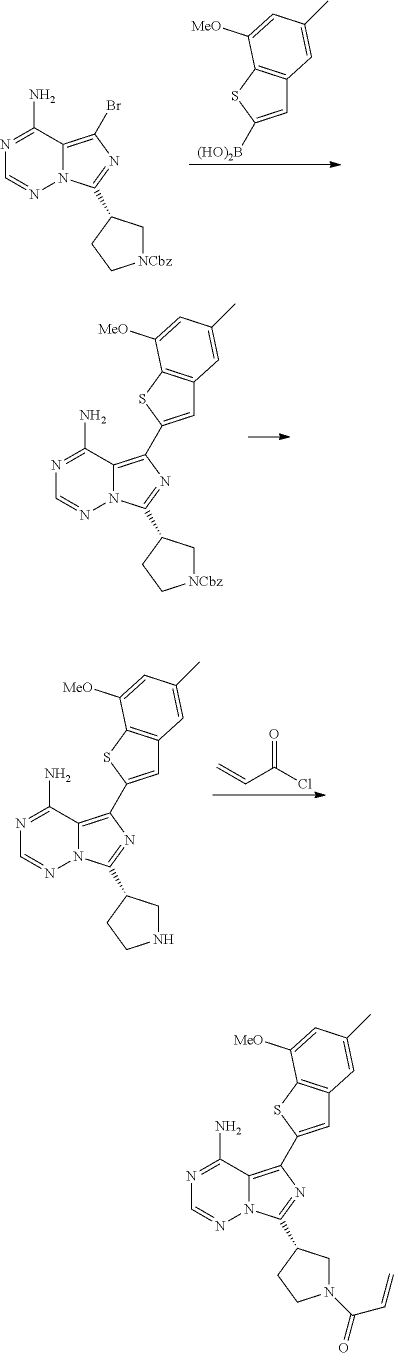

(S)-3-(((3-amino-5-oxo-4,5-dihydro-1,2,4-triazin-6-yl)methyl)carbamoyl)pyrrolidine-1-carboxylate (2.25 g, yield 67%) was obtained by the procedure analogous to that described in example 43 using 3-amino-6-(aminomethyl)-1,2,4-triazin-5(4H)-one hydrochloride as intermediate. LC/MS(ESI): m/z=433.1 [M+H]⁺.

Tert-butyl nitrite (0.77 g, 7.5 mmol), 10 mL of THF, several drops of DMF were added into the reaction flask, (S)-3-(2-amino-5-bromo-4-hydroxyimidazo[5,1-f][1,2,4]triazin-7-yl)pyrrolidine-1-carboxylate (2.17 g, 5 mmol) in THE (5 mL) was dropped. The mixture was stirred at room temperature for 12 h, the reaction mixture was concentrated under reduced pressure. The residue was purified through column chromatograph to obtain benzyl (S)-3-(5-bromo-4-hydroxyimidazo[5,1-f][1,2,4]triazin-7-yl)pyrrolidine-1-carboxylate (1.30 g, yield 62%) as a yellow solid. LC/MS(ESI): m/z=418.0 [M+H]⁺.

Benzyl (S)-3-(5-bromo-4-hydroxyimidazo[5,1-f][1,2,4]triazin-7-yl)pyrrolidine-1-carboxylate (1.25 g, 3 mmol) was dissolved in toluene (15 mL), POCl₃ (3.1 mL, 33 mmol) was added. The reaction mixture was heated, refluxed and reacted for 2 h, cooled to the room temperature and concentrated. The residue was poured into ice-water, extracted with DCM. The obtained organic phase was washed with saturated sodium bicarbonate and saturated NaCl aqueous solution, dried over anhydrous Na2SO4, the residue was purified through column chromatograph to obtain benzyl (S)-3-(5-bromo-4-chloroimidazo[5,1-f][1,2,4]triazin-7-yl)pyrrolidine-1-carboxylate (1.02 g, yield 78%). LC/MS(ESI): m/z=436.0[M+H]⁺. Compound 46 (80 mg, yield 37%) obtained by the procedures in the following 4 steps analogous to that described in example 185 was a yellow solid. ¹H NMR (400 MHz, DMSO-d₆) δ: 8.73 (br, 1H), 7.26-7.14 (d, 1H), 6.81 (s, 1H), 6.42-6.33 (m, 1H), 6.15-6.05 (m, 1H), 5.96-5.82 (br s, 2H), 5.71-5.63 (m, 1H), 4.18-3.95 (m, 3H), 3.91 (s, 3H), 3.64-3.53 (m, 2H), 2.49-2.30 (m, 5H); LC/MS(ESI): m/z=435.2 [M+H]⁺.

### Example 47

### Preparation for (S)-1-(3-(4-amino-5-(7-methoxy-5-methylbenzo[b]thiophen-2-yl)imidazo[5,1-f][1,2,4]triazin-7-yl)pyrrolidin-1-yl)but-2-yn-1-one (Compound 47)

(S)-5-(7-methoxy-5-methylbenzo[b]thiophen-2-yl)-7-(pyrrolidin-3-yl)imidazo[5,1-f][1,2,4]triazin-4-amine (190 mg, 0.5 mmol), triethylamine (76 mg, 0.75 mmol) and 2 mL of DCM were added into the reaction flask. 2-butynol chloride (78 mg, 0.75 mmol) in DCM (0.5 mL) was slowly dropped after an ice water bath. The reaction mixture was continuously stirred for 4 h after complete of addition. The reaction solution was quenched with methanol and concentrated. The residue was purified through column chromatograph to obtain compound 47 (100 mg, yield 45%) as a yellow solid. LC/MS(ESI): m/z=447.2 [M+H]⁺.

### Example 48

### 1-(1-acryloylazetidin-3-yl)-4-amino-3-(7-methoxy-5-methylbenzo[b]thiophen-2-yl)-1,6-dihydro-7H-pyrazolo[3,4-d]pyridazin-7-one (Compound 48)

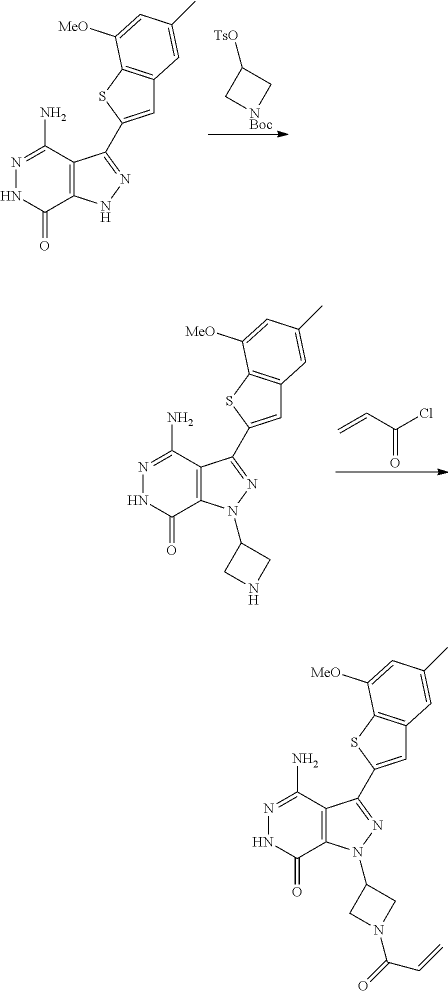

Tert-butyl 3-(tosyloxy)azetidine-1-carboxylate (392 mg, 1.2 mmol), PPh₃ (315 mg, 1.2 mmol) and 10 mL of THF were added into the reaction flask, DIAD (243 mg, 1.2 mmol) was added. The yellow solution was stirred for 5-10 minutes and then 4-amino-3-(7-methoxy-5-methylbenzo[b]thiophen-2-yl)-1,6-dihydro-7H-pyrazolo[3,4-d]pyridazin-7-one (310 mg, 1.0 mmol) was added. The mixture was stirred at room temperature for 12 h, evaporated to remove the solvent so as to obtain brown oil. The residue was purified through column chromatograph to obtain tert-butyl 3-(4-amino-3-(7-methoxy-5-methylbenzo[b]thiophen-2-yl)-7-oxo-6,7-dihydro-1H-pyrazolo[3,4-d]pyridazin-1-yl)azetidine-1-carboxylate (338 mg, yield 70%) as yellow solid. LC/MS(ESI): m/z=483.2[M+H]⁺.

The intermediate benzyl (S)-3-(8-amino-1-(7-methoxy-5-methylbenzo[b]thiophen-2-yl)imidazo[1,5-a]pyrazin-3-yl)pyrrolidine-1-carboxylate (296 mg, 0.6 mmol), 1 mL of 4 N HCl in dioxane and 1 mL of EtOAc were added into the reaction flask. The mixture was stirred at the room temperature for 2 h. The reaction mixture was neutralized with 1N NaOH solution, extracted with DCM. The obtained organic phase was washed with saturated sodium bicarbonate, and saturated brine, dried over anhydrous Na₂SO₄, concentrated under reduced pressure to give crude 4-amino-1-(azetidin-3-yl)-3-(7-methoxy-5-methylbenzo[b]thiophen-2-yl)-1,6-dihydro-7H-pyrazolo[3,4-d]pyridazin-7-one (220 mg, yield 96%). The crude was used in the next step directly. LC/MS(ESI): m/z=383.1[M+H]⁺

(S)-4-amino-3-(7-methoxy-5-methylbenzo[b]thiophen-2-yl)-1-(pyrrolidin-3-yl)-1,6-dihydro-7H-pyrrolo[2,3-d]pyridazin-7-one (191 mg, 0.5 mmol, triethylamine (76 mg, 0.75 mmol) and 2 mL of DCM were added into the reaction flask. The acryloyl chloride (78 mg, 0.75 mmol) in DCM (0.5 mL) was dropped after an ice water bath. The mixture was stirred continuously for 4 h after complete of addition. The reaction solution was quenched with methanol and concentrated under reduced pressure. The residue was purified through column chromatograph obtain to compound 48 (94 mg, yield 43%) as a yellow solid. LC/MS(ESI): m/z=437.1 [M+H]⁺.

### Example 49

### Preparation for 1-(1-acryloylazetidin-3-yl)-4-amino-3-(7-methoxy-5-methylbenzo[b]thiophen-2-yl)-1,6-dihydro-7H-pyrrolo[2,3-d]pyridazin-7-one (Compound 49)

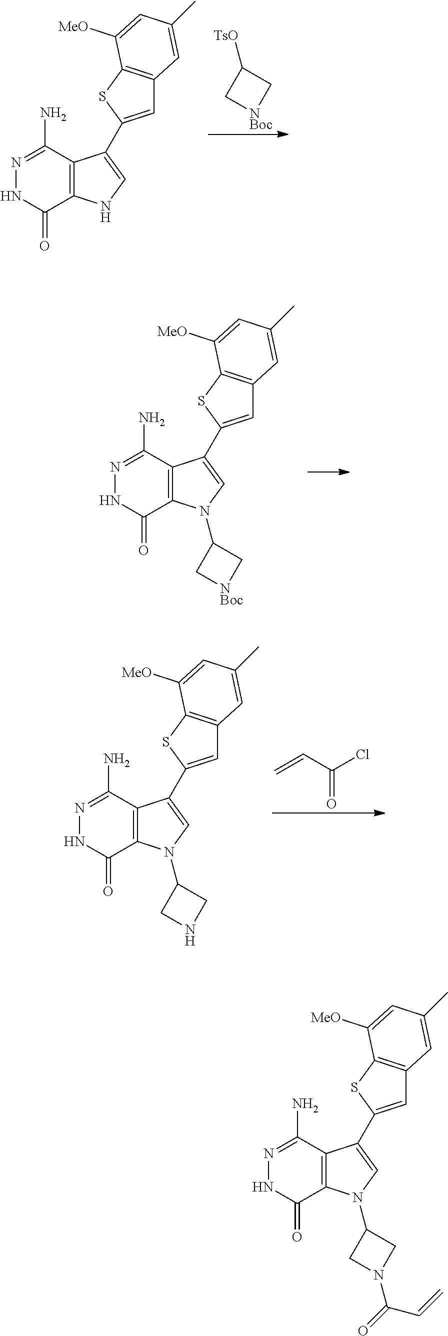

Compound 49 (92 mg, yield 42%) obtained by the procedure analogous to that described in example 48 with 4-amino-3-bromo-1,6-dihydro-7H-pyrrolo[2,3-d]pyridazin-7-one and (7-methoxy-5-methylbenzo[b]thiophen-2-yl)boronic acid as intermediate. 1-(1-acryloylazetidin-3-yl)-4-amino-3-(7-methoxy-5-methylbenzo[b]thiophen-2-yl)-1,6-dihydro-7H-pyrrolo[2,3-d]pyridazin-7-one was a yellow solid. LC/MS(ESI): m/z=436.1[M+H]⁺.

### Example 50

### Preparation for 1-(3-(8-amino-1-(7-methoxy-5-methylbenzo[b]thiophen-2-yl)imidazo[1,5-a]pyrazin-3-yl)azetidin-1-yl)prop-2-en-1-one (Compound 50)

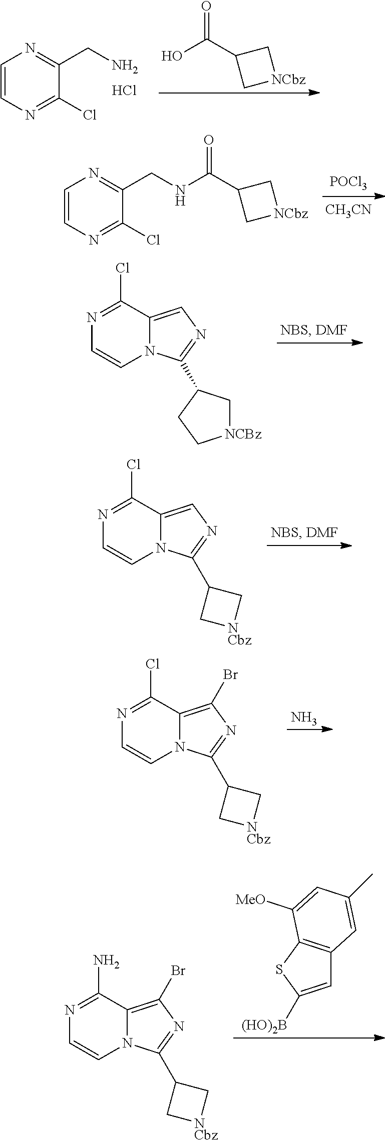

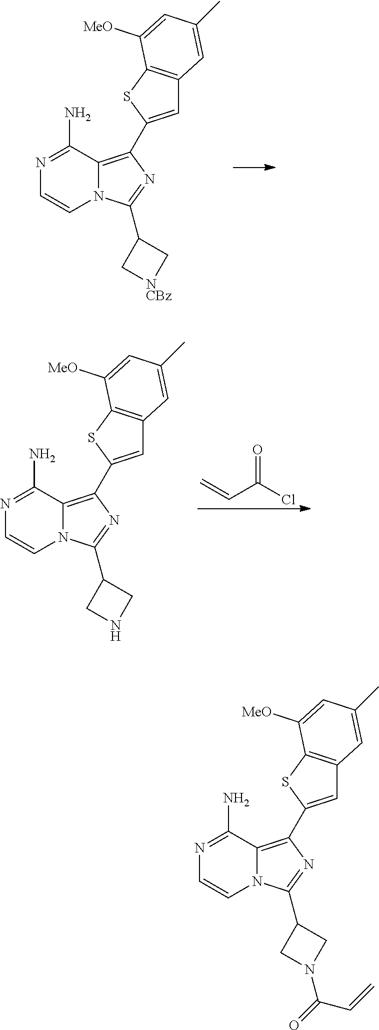

Compound 50 (67 mg, yield 32%) obtained by the procedure analogous to that described in example 46 using 1-((benzyloxy)carbonyl)azetidine-3-carboxylic acid as intermediate 1-(3-(8-amino-1-(7-methoxy-5-methylbenzo[b]thiophen-2-yl)imidazo[1,5-a]pyrazin-3-yl)azetidin-1-yl)prop-2-en-1-one was a yellow solid. LC/MS(ESI): m/z=420.1[M+H]⁺.

### Example 51

### Preparation for 1-(3-(4-amino-5-(7-methoxy-5-methylbenzo[b]thiophen-2-yl)imidazo[5,1-f][1,2,4]triazin-7-yl)azetidin-1-yl)prop-2-en-1-one (Compound 51)

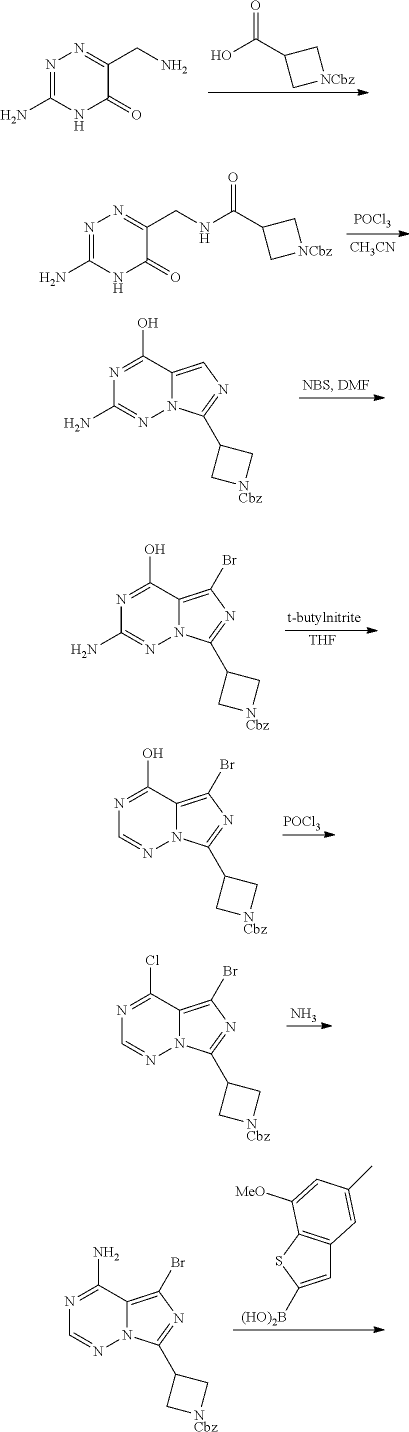

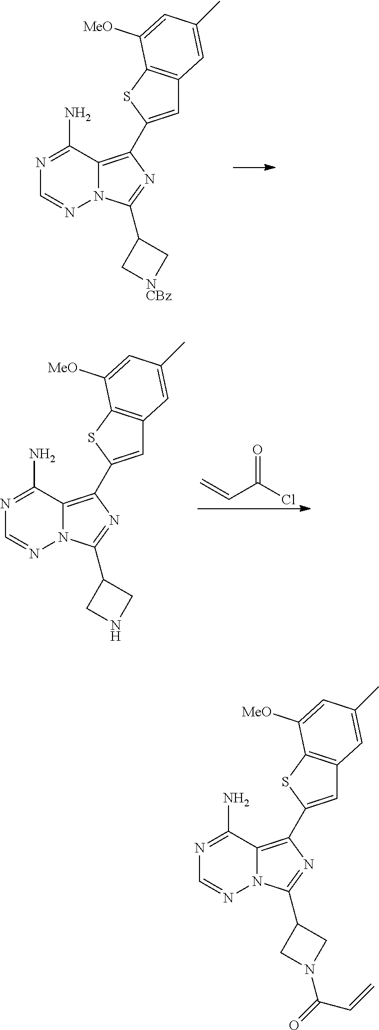

Compound 51 (74 mg, yield 350%) obtained by the procedure analogous to that described in example 46 using 1-((benzyloxy)carbonyl)azetidine-3-carboxylic acid as intermediate 1-(3-(4-amino-5-(7-methoxy-5-methylbenzo[b]thiophen-2-yl)imidazo[5,1-f][1,2,4]triazin-7-yl)azetidin-1-yl)prop-2-en-1-one was a yellow solid. LC/MS(ESI): m/z=421.1 [M+H]⁺.

### Example 52

### Preparation for 2-(1-acryloylazetidin-3-yl)-4-amino-6-(7-methoxy-5-methylbenzo[b]thiophen-2-yl)pyrimidine-5-carboxamide (Compound 52)

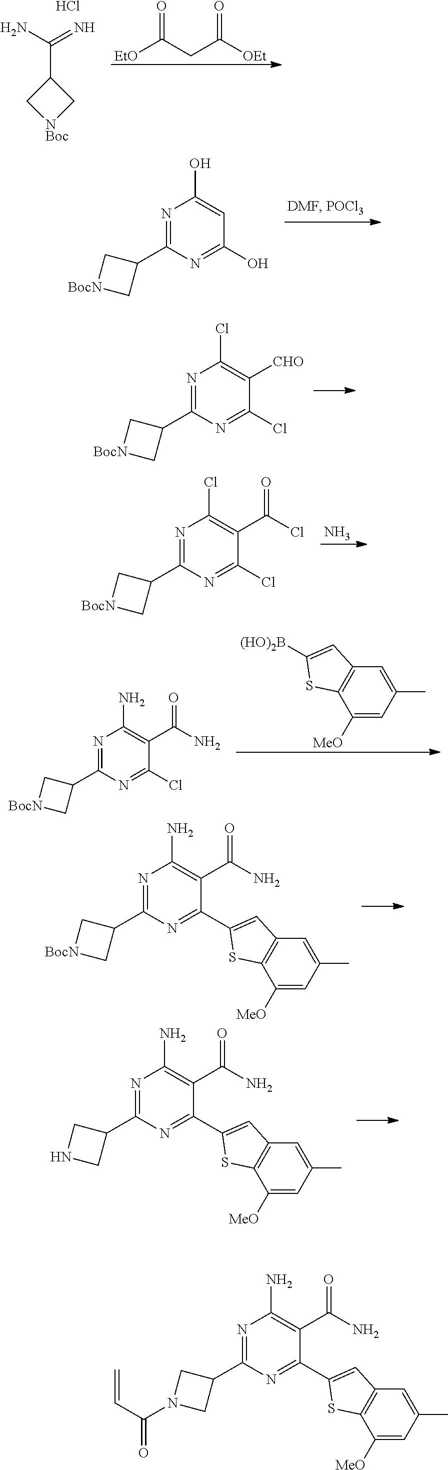

The mixture of tert-butyl 3-carbamimidoylazetidine-1-carboxylate hydrochloride (2.35 g, 10 mmol), NaOMe (2.16 g, 40 mmol) and 40 mL of MeOH were added into the reaction flask. The malonic acid (1.92 g, 12 mmol) in methanol (5 mL) was slowly added after an ice water bath. After addition, the mixture returned to the room temperature, was stirred and reacted for 12 h. The reaction solution was quenched with water, extracted with EtOAc and the organic phase was washed with saturated brine, dried over anhydrous Na₂SO₄, concentrated under reduced pressure. The residue was purified through column chromatograph to obtain tert-butyl 3-(4,6-dihydroxypyrimidin-2-yl)azetidine-1-carboxylate (2.22 g, yield 83%) as white solid. LC/MS(ESI): m/z=268.1 [M+H]⁺.

DMF (2 mL) and POCl₃ (6 mL) were added into the reaction flask. The mixture was stirred for 1 h under an ice water bath. The tert-butyl 3-(4,6-dihydroxypyrimidin-2-yl)azetidine-1-carboxylate (2.14 g, 8 mmol) was added. The reaction mixture was heated, refluxed and reacted for 4 h, cooled to room temperature and concentrated. The residue was poured into ice-water, extracted with DCM. The obtained organic phase was washed with saturated NaCl aqueous solution, dried over anhydrous Na₂SO₄, and the organic phase was evaporated under reduced pressure. The residue was purified through column chromatograph to obtain tert-butyl 3-(4,6-dichloro-5-formylpyrimidin-2-yl)azetidine-1-carboxylate (2.31 g, yield 87%) as yellow solid. LC/MS(ESI): m/z=332.1[M+H]⁺.

Tert-butyl 3-(4,6-dichloro-5-formylpyrimidin-2-yl)azetidine-1-carboxylate (1.66 g, 5 mmol), 20 mL of CCl₄, sulfonyl chloride (1.01 g, 7.5 mmol), azodiisobutyronitrile (41 mg, 0.25 mmol) were added into the reaction flask. The mixture was stirred at 80° C. for 4 h, cooled to room temperature, filtered and the filtrate was evaporated under reduced pressure to obtain crude tert-butyl 3-(4,6-dichloro-5-(chlorocarbonyl)pyrimidin-2-yl)azetidine-1-carboxylate (1.83 g, yield 100%) as yellow solid.

Tert-butyl 3-(4,6-dichloro-5-(chlorocarbonyl)pyrimidin-2-yl)azetidine-1-carboxylate (1.83 g, 5 mmol) and 20 mL of THE were added into the reaction flask and placed in an ammonia atmosphere. The mixture was stirred for 2 h at room temperature, concentrated under reduced pressure. The residue was diluted with water and EtOAc, extracted with EtOAc, washed with saturated brine and dried over anhydrous Na₂SO₄. The organic phase was evaporated to obtain crude tert-butyl 3-(4-amino-5-carbamoyl-6-chloropyrimidin-2-yl)azetidine-1-carboxylate (1.33 g, yield 81%) as a yellow solid. LC/MS(ESI): m/z=328.1[M+H]⁺.

Compound 52 (61 mg, yield 29%) obtained by the procedures in the following steps analogous to that described in example 46 2-(1-acryloylazetidin-3-yl)-4-amino-6-(7-methoxy-5-methylbenzo[b]thiophen-2-yl)pyrimidine-5-carboxamide as yellow solid. LC/MS(ESI): m/z=424.1[M+H]⁺.

### Example 53

### Preparation for 1-(1-acryloylazetidin-3-yl)-5-amino-3-(7-methoxy-5-methylbenzo[b]thiophen-2-yl)-1H-pyrazole-4-carboxamide (compound 53)

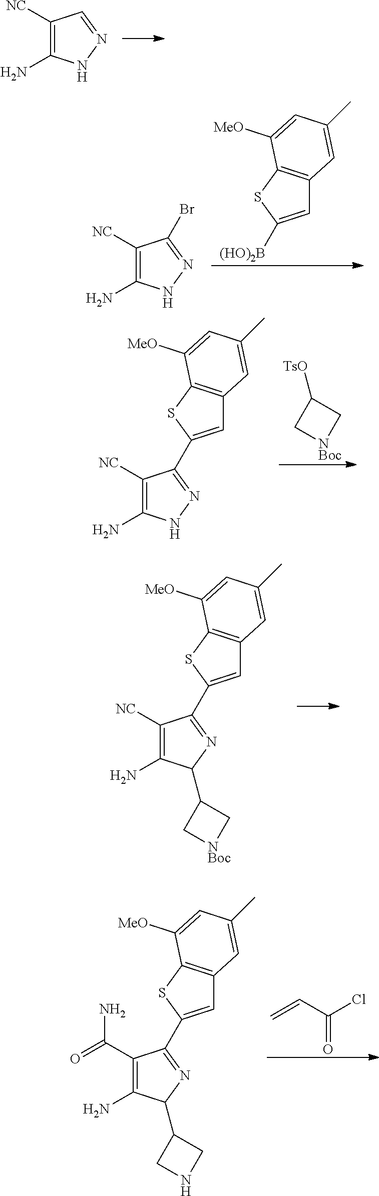

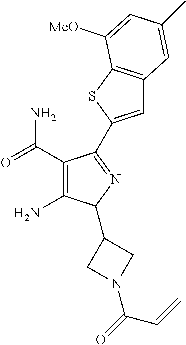 [SMILES: C=CC(=O)N1CC(C2N=C(c3cc4cc(C)cc(OC)c4s3)C(C(N)=O)=C2N)C1]

5-amino-3-(7-methoxy-5-methylbenzo[b]thiophen-2-yl)-1H-pyrazole-4-carbonitrile was obtained by the procedure analogous to that described in example 129 LC/MS(ESI): m/z=424.1[M+H]⁺.

5-amino-3-(7-methoxy-5-methylbenzo[b]thiophen-2-yl)-1H-pyrazole-4-carbonitrile (0.85 g, 2.0 mmol), 4 mL of EtOAc and 4N HCl in 4 mL of dioxane were added into the reaction flask. The mixture was stirred for 2 h at room temperature. The reaction mixture was neutralized with 1N NaOH solution, extracted with EtOAc. The obtained organic phase was washed with saturated sodium bicarbonate solution, dried over anhydrous Na2SO4. The organic phase was evaporated to obtain crude 5-amino-1-(azetidin-3-yl)-3-(7-methoxy-5-methylbenzo[b]thiophen-2-yl)-1H-pyrazole-4-carboxamide (0.61 g, yield 85%). LC/MS(ESI): m/z=358.1[M+H]⁺

5-amino-1-(azetidin-3-yl)-3-(7-methoxy-5-methylbenzo[b]thiophen-2-yl)-1H-pyrazole-4-carboxamide (179 mg, 0.5 mmol), triethylamine (76 mg, 0.75 mmol) and 2 mL of DCM were added into the reaction flask. A solution of acryloyl chloride (78 mg, 0.75 mmol) in DCM (0.5 mL) was slowly added after an ice water bath. The reaction mixture was stirred continuously for 4 h after completion of the addition. The reaction solution was quenched with methanol and concentrated under reduced pressure. The residue was purified through column chromatograph obtain compound 53 (68 mg, yield 33%) as yellow solid. LC/MS(ESI): m/z=412.1 [M+H]⁺.

### Example 54

### Preparation for (S)-2-(1-acryloylpyrrolidin-3-yl)-4-amino-6-(7-methoxy-5-methylbenzo[b]thiophen-2-yl)pyrimidine-5-carboxamide (Compound 54)

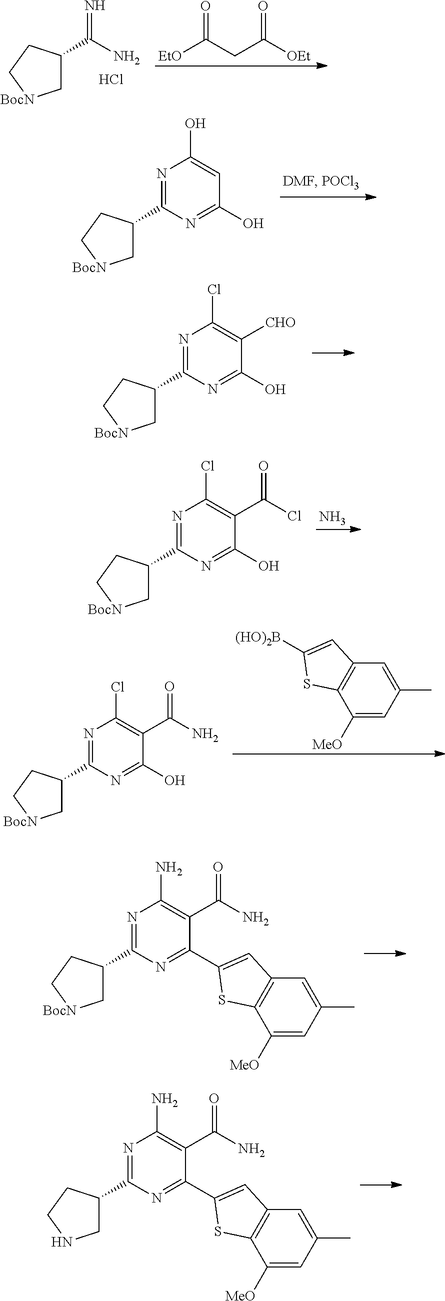

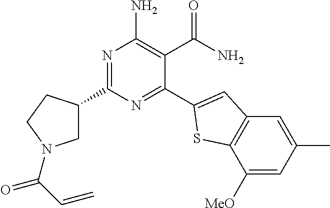 [SMILES: C=CC(=O)N1CC[C@H](c2nc(N)c(C(N)=O)c(-c3cc4cc(C)cc(OC)c4s3)n2)C1]

Compound 54 (83 mg, yield 38%) was obtained by the procedure analogous to that described in example 51 (S)-2-(1-acryloylpyrrolidin-3-yl)-4-amino-6-(7-methoxy-5-methylbenzo[b]thiophen-2-yl)pyrimidine-5-carboxamide as yellow solid. ¹H NMR (400 MHz, CD₃OD) δ: 7.69 (s, 1H), 7.21 (s, 1H), 6.73 (s, 1H), 6.32 (dd, 1H), 5.76 (dd, 1H), 5.02 (dd, 1H), 4.11-3.73 (m, 7H), 3.61-3.45 (m, 1H), 2.41-1.95 (m, 5H); LC/MS(ESI): m/z=438.2[M+H]⁺.

### Example 55

### Preparation for 1-(1-acryloylpyrrolidin-3-yl)-5-amino-3-(7-methoxy-5-methylbenzo[b]thiophen-2-yl)-1H-pyrazole-4-carboxamide (Compound 55)

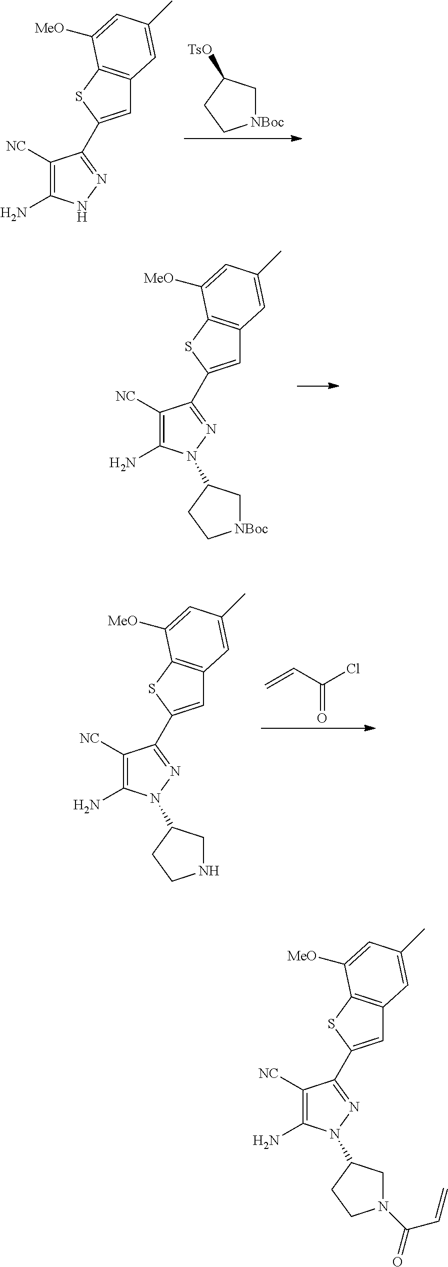

Compound 55 (87 mg, yield 41%) was obtained by the procedure analogous to that described in example 52 (S)-1-(1-acryloylpyrrolidin-3-yl)-5-amino-3-(7-methoxy-5-methylbenzo[b]thiophen-2-yl)-1H-pyrazole-4-carboxamide as yellow solid. ¹H NMR (400 MHz, CD₃OD) δ: 7.72 (s, 1H), 7.16 (s, 1H), 6.75 (s, 1H), 6.34 (dd, 1H), 5.75 (dd, 1H), 5.04 (dd, 1H), 4.11-4.03 (m, 1H), 3.97-3.71 (m, 6H), 3.59-3.45 (m, 1H), 2.39-1.93 (m, 5H); LC/MS(ESI): m/z=426.2[M+H]⁺.

### Example 56

In Vitro Activity Inhibition Test for Kinases FGFR1, FGFR2, FGFR3 and FGFR4

The activity of FGFR1, FGFR2, FGFR3 and FGFR4 protein kinases was determined by Caliper mobility shift assay. Perform a 4-fold gradient dilution from a working concentration of 0.2 mM in DMSO, diluting 10 concentrations. Add 2 μL of compound to 78 μL of 1× compound buffer. There were 10 points each for the negative control and the positive control. Shake the board on the rocker for 20 min. Transfer 2 μL of kinase to the 384 plate and add 1 μL of the compound to be tested to the 384 plate, centrifuge at 1000 rpm/min and incubate at 25° C. for 10 min. Transfer 2 μL of the substrate mixture to a 384 plate, centrifuge at 1000 rpm/min, and incubate at 25° C. for 50 min. The final concentration of DMSO was 0.5%. Prepare 2×Sa-XL 665/TK-antibody-Cryptate mix with HTRF detection buffer. Add 5 μL of Sa-XL 665/TK-antibody-Cryptate per well, Centrifuge at 1,000 rpm/m for 30 sec and react for 1 h at room temperature. Read the fluorescence signals at 615 nm (Cryptate) and 665 nm (XL665) with BMG. Convert conversion rate into inhibition data (% inhibition rate=(max-sample conversion rate)/(max-min)*100 where max refers to the conversion rate of DMSO control and min refers to the conversion rate of the enzyme-free live control. Using compound concentration and inhibition as the abscissa and ordinate coordinates, plot the curve, fit the curve using Graphpad software, and calculate IC50. The assay results are shown in the table below showing the activity data of compounds 1-54 for the kinases FGFR1, FGFR2, FGFR3, and FGFR4. Activity is characterized by IC50, where “A” denotes IC50≤10 nM; “B” means 10<IC50≤100 nM; “C” stands for IC50≤500 nM<100; “D” stands for 500<IC50≤2000 nM.

Inhibition of separase IC₅₀ (nM)

| NO | FGFR1 | FGFR2 | FGFR3 | FGFR4 |
|---|---|---|---|---|
| 1 | B | B | C | B |
| 2 | B | B | C | C |
| 3 | B | B | B | B |
| 4 | B | C | B | B |
| 5 | B | B | C | B |
| 6 | A | A | A | A |
| 7 | B | B | B | B |
| 8 | C | B | B | C |
| 9 | C | B | B | C |
| 10 | B | B | C | B |
| 11 | B | B | C | B |
| 12 | A | A | A | A |
| 13 | B | B | B | C |
| 14 | B | B | C | B |
| 15 | A | A | A | A |
| 16 | A | A | B | B |
| 17 | A | A | A | A |
| 18 | B | A | A | A |
| 19 | A | A | A | A |
| 20 | A | A | A | A |
| 21 | A | A | A | A |
| 22 | A | A | A | A |
| 23 | A | A | A | A |
| 24 | A | A | A | A |
| 25 | A | A | A | A |
| 26 | A | A | A | A |
| 27 | A | A | A | A |
| 28 | A | A | A | A |
| 29 | A | A | A | A |
| 30 | A | A | A | A |
| 32 | A | A | A | A |
| 34 | A | A | A | A |
| 36 | A | A | A | A |
| 38 | A | A | A | A |
| 39 | A | A | A | A |
| 40 | A | A | A | A |
| 41 | A | A | A | A |
| 42 | A | A | A | A |
| 43 | A | A | A | A |
| BGJ398 | A | A | A | B |
| Futibatinib | A | A | A | A |

Conclusion: most of the compounds of the present invention have strong inhibitory activity on FGFR1-4, and the inhibitory activity reaches less than 10 nm. Some of these compounds have inhibitory activity of FGFR1-4 to less than 1 nm.

### Example 57: Human Hepatoma Cell Hep3B Survival Test

The Hep3B cell line of human liver cancer is derived from ATCC. Cells are supplemented with McCoy's 5A medium and additionally added fetal bovine serum (IOFBS). Cells are kept in the medium at 37° C., humidity at 95%, and carbon dioxide at 500. Hep3B cells were seeded in 96-well plates at a density of 3500 cells per well with a cell suspension volume of 90 μL per well and cultured at 37° C. in a cell culture incubator containing 500 CO₂. The next day, the final concentration of the test compound was 1 μM (as the starting concentration of the IC₅₀ test), quadrupled and diluted by 9 concentrations. The 9 concentrations are: 1l μM, 2.5 μM, 0.625 μM, 0.156 μM, 0.039 μM, 0.0098 μM, 0.0024 μM, 0.0006 μM and 0.000015 μM, mix and centrifuge, add 1PL compound DMSO solution to the cell culture medium, and use 1M DMSO as a control, with three parallel side wells for each concentration of each compound. The cells were then placed in a 37° C. incubator and treated with compounds for 72 hours. Add 50 μL of CellTiter-Glo (Promega, Madison WI) to the cell culture medium, and determine the relative luminescence unit (RLU) of each well and calculate cell viability and compound activity (IC₅₀), “A” means IC₅₀≤10 nM; “B” means 10<IC₅₀≤100 nM; “C” means 100<IC₅₀≤500 nM; “D” means 500<IC₅₀≤2000 nM. The results of the inhibitory activity of the embodiment compound on Hep3B cells are shown in Table 2 below:

TABLE 2
inhibitory activity on Hep3B cell proliferation

| Sample No. | IC₅₀ (nM) |
|---|---|
| 20 | A |
| 22 | A |
| 24 | A |
| 26 | A |
| 28 | A |
| 30 | A |
| 32 | A |
| 34 | A |
| 36 | A |
| 38 | A |
| Pemigatinib | B |
| infigratinib | B |
| Futibatinib | A |

### Example 58: Evaluation of Proliferative Inhibition Activity of Human Gastric Cancer Cells and Bladder Cancer Cells

Using the CellTiter-Glo<TM>Live Cell Assay Kit, The inhibitory effect of the test compound on the proliferation of human gastric cancer cells (SNU-16) and FGFR3 high expression and FGFR3 fusion of FGFR3-TACC3 fused human bladder cancer cells (RT4) was determined. Among them, RT4 medium is added with fetal bovine serum and McCoy's 5A medium with a final concentration of 10%.

Test steps: SNU-16 and RT4 cells that have reached 80% cell confluency were digested by trypsin, centrifuged and re-suspended for counting, and 3500 and 6000 cells/mL of SNU-16 and RT4 cell suspensions were prepared with medium, respectively. Add a 96-well cell culture plate (90 μL/well) and place in a cell culture incubator containing 5% CO₂ at 37° C. After 24 hours of cell culture, the reference compound table and tested compound A were dissolved with DMSO into a mother liquor with a concentration of 30 mM. The diluted compound stock solution was further diluted with SNU-16 and RT4 medium and the diluted mixture was transferred to the corresponding cell plates, respectively. The final concentration of the test compound was 1 μM (as the starting concentration for the IC₅₀ test), quadruple decreasing dilution at 9 concentrations. They are: 1 μM, 2.5 μM, 0.625 μM, 0.156 μM, 0.039 μM, 0.0098 μM, 0.0024 μM, 0.0006 μM and 0.000015 μM. Mix and centrifuge and then place in a cell culture incubator containing 5% CO₂ for 3 days at 37° C. Take out the 96-well cell culture plate and add CellTiterGlo (CTG, Chemiluminescent Cell Viability Assay) reagent (100 L/well. Mix and centrifuge, and then incubate for 10 min at room temperature. After gentle shaking, the absorbance at 450 nm wavelength was determined on the SpectraMax M5 Reader, and the absorbance at 650 nm was used as a reference (i.e., 450 nm absorbance −650 nm absorbance) to calculate the suppression.

TABLE 2
Inhibition of proliferation of SNU-16 and RT4 cells IC₅₀ (nM)

| Sample Code | SNU-16 | RT4 |
|---|---|---|
| 28 | 0.025 | 1.3 |
| 38 | <0.015 | 3.4 |
| Pemigatinib | 0.249 | 6.0 |
| infigratinib | 0.572 | 11.8 |
| Futibatinib | 0.022 | 12.6 |
| Erdafinib | ND | 1.8 |

Conclusion: the compounds have strong inhibitory activity on the proliferation of human gastric cancer cells (SNU-16), human bladder cancer cells (RT4) and human liver cancer Hep3B cells. Some compounds have stronger inhibitory activity than control compounds such as Pemigatinib, infigratinib, Futibatinib and Erdafinib.

### Example 59: Determination of Potassium Channel Blockade of hERG

The experimental methods are in follows:

extracellular fluid: 140 mM NaCl, 3.5 mM KCl, 1 mM MgCl₂, 2 mM CaCl₂, 10 mM D-glucose, 10 mM HEPES, 1.25 mM NaH₂PO₄, pH=7.40

ISE Internal Solution: 20 mM KCl, 115 mM K-aspartate, 1 mM MgCl₂, 5 mM EGTA, 10 mM HEPES, 2 mM Na2-ATP, pH=7.2

Cell culture: HEK293 cell line with stable expression of hERG potassium channel was used, and hERG potassium channel cells were purchased from Creacell (catalog number: A-0320). It was cultured in DMEM medium containing 10 fetal bovine serum and 0.8 mg/mL G418 at 37° C. and a carbon dioxide concentration of 5%. Take out the old medium and wash once with PBS, then add 2 mL of TrypLE™ Express solution and incubate at 37° C. for about 1 min. When the cell detaches from the bottom of the dish, add approximately 5 mL of complete medium pre-warmed at 37° C. Gently pipette the cell suspension with a pipette to detach the aggregated cells. Transfer the cell suspension to a sterile centrifuge tube and centrifuge at 1,000 rpm for 5 min to collect the cells. Expand or maintain cultures and seed cells in 10 cm cell dishes with a cell volume of 6105 cells per dish (final volume: 10 mL). To maintain the electrophysiological activity of cells, the cell density should not exceed 80%.

The voltage stimulation protocol for whole-cell patch clamp recording whole-cell hERG potassium current is as follows: cell membrane voltage clamping at −80 mV after formation of whole-cell sealing. The tail current of the hERG channel can be excited by clamping the clamping voltage from −80 mV to −50 mV for 0.5 s (as leakage current sensing), then stepping to 30 mV for 2.5 s and quickly recovering to −50 mV for 4 s. Data were collected repeatedly every 10 s to observe the effect of the drug on the hERG tail current. A −50 mV stimulus of 0.5 s was used as a leakage current detection. Test data is collected by Qpatch and stored at a connected service station.

Each drug concentration is set for two administrations for at least 5 minutes. The tested compound and the compound-free external fluid acts on the cells sequentially from low to high concentration, and each cell uses the current detected in the compound-free exo-liquid as its own control group, and the detection of the two cells is repeated independently. All electrophysiological tests are performed at 24° C.

First, the current after action of each drug concentration and the blank control current are normalized (Peak tail current compoundPeak tail current vehicle). The inhibition rate corresponding to each drug concentration (1-(Peak tail current compound)/(Peak tail current vehicle) is then calculated. Calculate the mean and standard error for each concentration, and calculate the semi-inhibitory concentration for each compound using the following equation:

Y=Bottom+(Top−Bottom)/(1+10\hat{ }((Log IC₅₀−X)*HillSlope)).

The dose-dependent effect is non-linear fitted with the equation, where C represents the concentration of the test substance, IC₅₀ is the semi-inhibitory concentration, and h represents the Hill coefficient. Curve fitting and IC₅₀ calculations are done using Graphpad software.

The test results shows that the test substance 28 is of weak or no inhibition effect on the hERG channel. Substance 38 is of a moderate inhibitory effect on the hERG channel.

Although the present disclosure is described in detail above, those skilled in the art may understand that various modifications and changes may be made to the present disclosure under a premise of without deviating from the spirt and scope of the present disclosure. The claim scope of the present disclosure is not limited to the above detail description, but belongs to the claims.

1. A compound represented by general formula (I), a stereoisomer thereof, a pharmaceutical salt, a polymorph or an isomer, wherein the structure of the compound represented by the general formula (I) is as follows: 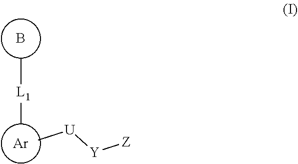
in the formula,
each ring B is a benzyl or a 5-10 membered heteroaryl, and the above benzyl and heteroaryl are optionally substituted with one or more G¹;
each L₁ is independently selected from bonds, —C₁₋₄ alkyl-, —C₂₋₄ alkenyl-, —C₂₋₄ alkynyl-;
each aromatic ring Ar is 6-10 membered heteroaromatic ring, the benzyl ring and heteroaromatic ring described above are optionally substituted with one or more R¹;
each R¹ is independently selected from H, D, cyano, halogen, C₁₋₆ alkyl, C₃₋₆ cycloalkyl, 3-6-membered heteroalkyl, —OR², —NR²R³, —C(O)NR²R³, and the alkyl, cycloalkyl or heteroalkyl are optionally substituted with cyano, halogen, —OR⁴, —NR⁴R⁵, C₁₋₆ alkyl, C₃₋₆ cycloalkyl or 3-6 heteroalkyl;
each U is independently selected from —C₀₋₄ alkyl-, —CR⁶R⁷—, —C₁₋₂ alkyl(R⁶)(OH)—, —C(O)—, —CR⁶R⁷O—, —OCR⁶R⁷—, —SCR⁶R⁷—, —CR⁶R⁷S—, —NR⁶—, —NR⁶C(O)—, —C(O)NR⁶—, —NR⁶C(O)NR⁷—, —CF₂—, —O—, —S—, —S(O)ₘ—, —NR⁶S(O)₂—, —S(O)₂NR⁶—;
each Y is absent or selected from C₃₋₈ cycloalkyl, 3-8-membered heterocycloalkyl, 5-12 thick alkyl, 5-12 thick heterocyclyl, 5-12 membered spirocyclyl, 5-12 membered spirocyclyl, aryl or heteroaryl, 3-8 heterocycloalkyl, 5-12 membered thick heterocyclyl, 5-12 membered spiroheterocyclyl or heteroaryl independently comprises 1, 2, 3, or 4 heteroatoms selected from N, O, or S at each occurrence, and the cycloalkyl, heterocyclyalkyl, spirocyclyl, polycycle, heteropolycyclyl, heterospirocyclyl, aryl, or heteroaryl optionally is substituted with one or more G²;
each Z is independently selected from cyano, —NR⁸CN, 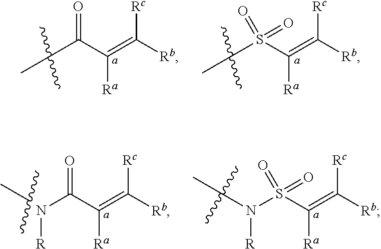
bond a is a double bond or a triple bond;
when a is a double bond, each of R^{a}, R^{b} and R^{c} is independently selected from H, D, cyano, halogen, C₁₋₆ alkyl, C₃₋₆ cycloalkyl or 3-6 heterocyclyl, and the alkyl, cycloalkyl and heterocyclyl are optionally substituted one or more G³;
each of R^{a} and R^{b} or R^{b} and R^{c} optionally forms an optional 3-6-membered ring containing heteroatoms with carbon atoms attached to them;
when a is a triplet bond, R^{a} and R^{c} are absent, each R^{b} s independently selected for H, D and cyano, and halogen, C₁₋₆ alkyl, C₃₋₆ cycloalkyl or 3-6 heterocyclyl is substituted with one or more G⁴;
each R⁸ is independently selected from H, D, C₁₋₆ alkyl, C₃₋₆ cycloalkyl or 3-6-membered heterocyclyl, and the alkyl, cycloalkyl and heterocyclyl are optionally substituted with one or more G⁵;
each of G¹, G², G³, G⁴ and G⁵ is independently selected from H, D, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR⁹, —OC(O)NR⁹R¹⁰, —C(O)OR⁹, —C(O)NR⁹R¹⁰, —C(O)R⁹, —NR⁹R¹⁰, —NR⁹C(O)R¹⁰, —NR⁹C(O)NR¹⁰R¹¹, —S(O)ₘR⁹ or —NR⁹S(O)ₘR¹⁰, the alkyl, alkenyl, alkynyl, cycloalkyl, heteroalkyl, aryl and heteroaryl are independently substituted with substituents of one or more cyano, halogen, C₁₋₆alkyl, C₂₋₆alkenyl, C₂₋₆ alkynyl, C₃₋₈ heteroalkyl or 3-8 membered heterocyclyl, C₆₋₁₀ aryl, 5-10 membered heteroaryl, —OR¹², —OC(O)NR¹²R¹³, —C(O)OR¹², —C(O)NR¹²R¹³, —C(O)R¹², —NR¹²R¹³, —NR¹²C(O)R¹³, —NR¹²C(O)NR¹³R¹⁴, S(O)ₘR¹² or —NR¹²S(O)ₘR¹³;
each of R³, R⁴, R⁵, R⁶, R⁷, R⁸, R⁹, R¹¹, R¹², R¹³ and R¹⁴ is independently selected from H, D, cyano, halogen, C₁₋₆ alkyl, C₃₋₈ cycloalkyl or 3-8 membered monocyclic heterocycyl, monocyclic heteroaryl or phenyl; and
m is 1 or 2.

2. The compound represented by general formula (I), the pharmaceutically acceptable salt thereof or the stereoisomer thereof according to claim 1, wherein the general formula (I) is further represented by formula IIa: 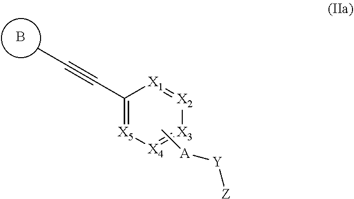
in the formula,
each of X₁, X₂, X₃, X₄, X₅ is independently selected from CR¹ or N, and at least one of X₁, X₂, X₃, X₄, X₅ is N;
each R¹ is independently selected from H, D, cyano, halogen, C₁₋₆ alkyl, C₃₋₆ cycloalkyl, 3-6 membered heteroalkyl, —OR², —NR²R³, —C(O)NR²R³, and the alkyl, cycloalkyl or heteroalkyl is optionally substituted with cyano, halogen, —OR⁴, —NR⁴R⁵, C₁₋₆ alkyl, C₃₋₆ cycloalkyl or 3-6 membered heterocyclyl;
each ring B is a benzyl or a 5-6 membered heteroaryl, and the aryl and heteroaryl described above are optionally substituted with one or more G¹;
each U is independently selected from —C₀₋₄ alkyl-, —CR⁶R⁷—, —C₁₋₂ alkyl (R⁶)(OH)—, —C(O)—, —CR⁶R⁷O—, —OCR⁶R⁷—, —SCR⁶R⁷—, —CR⁶R⁷S—, —NR⁶—, —NR⁶C(O)—, —C(O)NR⁶—, —NR⁶C(O)NR⁷—, —CF₂—, —O—, —S—, —S(O)ₘ—, —NR⁶S(O)₂—, —S(O)₂NR⁶—;
each Y is absent or selected from C₃₋₈ cycloalkyl, 3-8-membered heterocycloalkyl, 5-12 thick alkyl, 5-12 thick heterocyclyl, 5-12 membered spirocyclyl, 5-12 membered spirocyclyl, aryl or heteroaryl, 3-8 heterocycloalkyl, 5-12 membered thick heterocyclyl, 5-12 membered spiroheterocyclyl or heteroaryl independently comprises 1, 2, 3, or 4 heteroatoms selected from N, O, or S at each occurrence, and the cycloalkyl, heterocyclyalkyl, spirocyclyl, polycycle, heteropolycyclyl, heterospirocyclyl, aryl or heteroaryl is optionally substituted with one or more G²;
each Z is independently selected from cyano, —NR⁸CN, 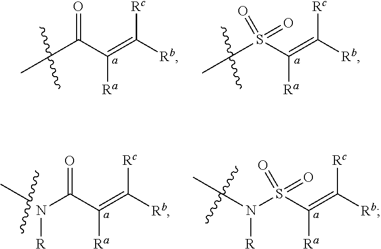
bond a is a double bond or a triple bond;
when a is a double bond, each of R^{a}, R^{b} and R^{c} is independently selected from H, D, cyano, halogen, C₁₋₆ alkyl, C₃₋₆ cycloalkyl or 3-6 heterocyclyl, and the alkyl, cycloalkyl and heterocyclyl are optionally substituted one or more G³;
each of R^{a} and R^{b} or R^{b} and R^{c} optionally forms an optional 3-6-membered ring containing heteroatoms with carbon atoms attached to them;
when a is a triplet bond, R^{a} and R^{c} are absent, each R^{b} is independently selected for H, D and cyano, and the halogen, C₁₋₆ alkyl, C₃₋₆ cycloalkyl or 3-6 heterocyclyl is substituted with one or more G⁴;
each R⁸ is independently selected from H, D, C₁₋₆ alkyl, C₃₋₆ cycloalkyl or 3-6-membered heterocyclyl, and the alkyl, cycloalkyl and heterocyclyl are optionally substituted with one or more G⁵;
each of G¹, G², G³, G⁴ and G⁵ is independently selected from H, D, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR⁹, —OC(O)NR⁹R¹⁰, —C(O)OR⁹, —C(O)NR⁹R¹⁰, —C(O)R⁹, —NR⁹R¹⁰, —NR⁹C(O)R¹⁰, —NR⁹C(O)NR¹⁰R¹¹, —S(O)ₘR⁹ or —NR⁹S(O)ₘR¹⁰, and the alkyl, alkenyl, alkynyl, cycloalkyl, heteroalkyl, aryl, heteroaryl is independently substituted with substituents of one or more cyano, halogen, C₁₋₆ alkyl, C₂₋₆ alkenyl, C₂₋₆ alkynyl, C₃₋₈ heteroalkyl or 3-8 membered heterocyclyl, C₆₋₁₀ aryl, 5-10 membered heteroaryl, —OR¹², —OC(O)NR¹²R¹³, —C(O)OR¹², —C(O)NR¹²R¹³, —C(O)R¹², —NR¹²R¹³, —NR¹²C(O)R¹³, —NR¹²C(O)NR¹³R¹⁴, S(O)ₘR¹² or —NR¹²S(O)ₘR¹³;
each of R³, R⁴, R⁵, R⁶, R⁷, R⁸, R⁹, R¹¹, R¹², R¹³ and R¹⁴ is independently selected from H, D, cyano, halogen, C₁₋₆ alkyl, C₃₋₈ cyclyl alkyl or 3-8 membered monocyclic heterocycyl, monocyclic heteroaryl or phenyl; and
m is 1 or 2.

3. The compound represented by general formula (I), the pharmaceutically acceptable salt thereof or the stereoisomer thereof according to claim 1, wherein the general formula (I) is further represented by formula IIf: 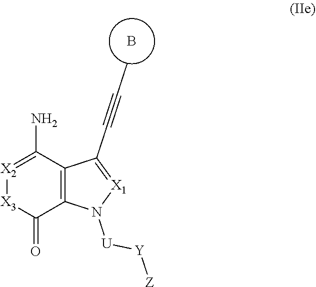
in the formula,
each of X₁, X₂, X₃, X₄, X₅ is independently selected from N, CR¹;
each R¹ is independently selected from H, D, cyano, halogen, C₁₋₆ alkyl, C₃₋₆ cycloalkyl, 3-6 membered heteroalkyl, —OR², —NR²R³, —C(O)NR²R³, and the alkyl, cycloalkyl or heteroalkyl is optionally substituted with cyano, halogen, —OR⁴, —NR⁴R⁵, C₁₋₆ alkyl, C₃₋₆ cycloalkyl or 3-6 membered heterocyclyl;
each ring B is a benzyl or a 5-6 membered heteroaryl, and the aryl and heteroaryl described above are optionally substituted with one or more G¹;
each U is independently selected from —C₀₋₄ alkyl-, —CR⁶R⁷—, —C₁₋₂ alkyl(R⁶)(OH)—, —C(O)—, —CR⁶R⁷O—, —OCR⁶R⁷—, —SCR⁶R⁷—, —CR⁶R⁷S—, —NR⁶—, —NR⁶C(O)—, —C(O)NR⁶—, —NR⁶C(O)NR⁷—, —CF₂—, —O—, —S—, —S(O)ₘ—, —NR⁶S(O)₂—, —S(O)₂NR⁶—;
each Y is absent or selected from C₃₋₈ cycloalkyl, 3-8-membered heterocycloalkyl, 5-12 thick alkyl, 5-12 thick heterocyclyl, 5-12 membered spirocyclyl, 5-12 membered spirocyclyl, aryl or heteroaryl; 3-8 heterocycloalkyl, 5-12 membered thick heterocyclyl, 5-12 membered spiroheterocyclyl or heteroaryl independently comprises 1, 2, 3, or 4 heteroatoms selected from N, O, or S at each occurrence; and the cycloalkyl, heterocyclyalkyl, spirocyclyl, polycyclic, heteropolycyclyl, heterospirocyclyl, aryl or heteroaryl is optionally substituted with one or more G²;
each Z is independently selected from cyano, —NR⁸CN, 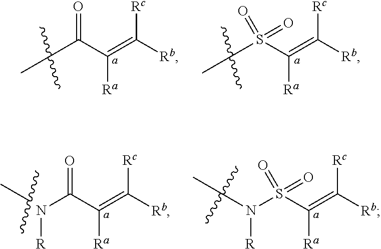
bond a is a double bond or a triple bond;
when a is a double bond, each of R^{a}, R^{b} and R^{c} is independently selected from H, D, cyano, halogen, C₁₋₆ alkyl, C₃₋₆ cycloalkyl or 3-6 heterocyclyl, and the alkyl, cycloalkyl and heterocyclyl are optionally substituted one or more G³;
each of R^{a} and R^{b} or R^{b} and R^{c} optionally forms an optional 3-6-membered ring containing heteroatoms with carbon atoms attached to them;
when a is a triplet bond, R^{a} and R^{c} are absent, each R^{b} is independently selected for H, D and cyano, and the halogen, C₁₋₆ alkyl, C₃₋₆ cycloalkyl or 3-6 heterocyclyl is substituted with one or more G⁴;
each R⁸ is independently selected from H, D, C₁₋₆ alkyl, C₃₋₆ cycloalkyl or 3-6-membered heterocyclyl, and the alkyl, cycloalkyl and heterocyclyl are optionally substituted with one or more G⁵;
each of G¹, G², G³, G⁴ and G⁵ is independently selected from H, D, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR^{9′}—OC(O)NR⁹R¹⁰, —C(O)OR⁹, —C(O)NR⁹R¹⁰, —C(O)R⁹, —NR⁹R¹⁰, —NR⁹C(O)R¹⁰, —NR⁹C(O)NR¹⁰R¹¹, —S(O)ₘR⁹ or —NR⁹S(O)ₘR¹⁰; and the alkyl, alkenyl, alkynyl, cycloalkyl, heteroalkyl, aryl, heteroaryl is independently substituted with substituents of one or more cyano, halogen, C₁₋₆ alkyl, C₂₋₆ alkenyl, C₂₋₆ alkynyl, C₃₋₈ heteroalkyl or 3-8 membered heterocyclyl, C₆₋₁₀ aryl, 5-10 membered heteroaryl, —OR¹², —OC(O)NR¹²R¹³, —C(O)OR¹², —C(O)NR¹²R¹³, —C(O)R¹², —NR¹²R¹³, —NR¹²C(O)R¹³, —NR¹²C(O)NR¹³R¹⁴, S(O)ₘR¹² or —NR¹²S(O)ₘR¹³;
each of R³, R⁴, R⁵, R⁶, R⁷, R⁸, R⁹, R¹¹, R¹², R¹³ and R¹⁴ is independently selected from H, D, cyano, halogen, C₁₋₆ alkyl, C₃₋₈ cyclyl alkyl or 3-8 membered monocyclic heterocycyl, monocyclic heteroaryl or phenyl; and
m is 1 or 2.

4. The compound represented by general formula (I), the pharmaceutically acceptable salt thereof or the stereoisomer thereof according to claim 1, wherein the general formula (I) is further represented by formula IIg: 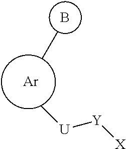
in the formula,
the ring Ar is a 5-10 membered heteroaryl, and the above 5-10 membered heteroaryl is optionally substituted with one or more G¹;
the ring B is independently selected from 5-14 heteroaryl and 5-14 aryl containing 1-3 heteroatoms selected from S, O, N and Se, and the 5-14 heteroaryl described above is substituted with one or more G²;
each U is dependently selected from —C₀₋₄ alkyl-, —CR⁷R⁸—, —C₁₋₂alkyl (R⁷)(OH)—, —C(O)—, —CR⁷R⁸O—, —OCR⁷R⁸—, —SCR⁷R⁸—, —CR⁷R⁸S—, —NR⁷—, —NR⁷C(O)—, —C(O)NR⁷—, —NR⁷C(O)NR⁸—, —CF₂—, —O—, —S—, —S(O)ₘ—, —NR⁷S(O)₂—, —S(O)₂NR⁷—;
Y is absent or selected from C₃₋₈ cycloalkyl, 3-8-membered heterocycloalkyl, 5-12 thick alkyl, 5-12 thick heterocyclyl, 5-12 membered spirocyclyl, 5-12 membered spiroheterocyclyl, aryl or heteroaryl, and the cycloalkyl, heterocyclyl, spirocyclyl, thickcyclyl, thickheterocyclyl, spiroheterocyclyl, aryl, heteroaryl is optionally substituted with one or more G³;
Z is independently selected from cyano, NR⁹CN, 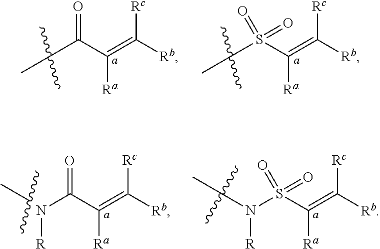
when bond a is a double bond, each of R^{a}, R^{b} and R^{c} is independently selected from H, D, cyano, halogen, C₁₋₆ alkyl, C₃₋₆ cycloalkyl or 3-6 heterocyclyl, and the alkyl, cycloalkyl and heterocyclyl are optionally substituted one or more G³;
each of R^{a} and R^{b} or R^{b} and R^{c} optionally forms an optional 3-6-membered ring containing heteroatoms with carbon atoms attached to them;
when bond a is a triplet bond, R^{a} and R^{c} are absent, each R^{b} is independently selected for H, D, and cyano, and the halogen, C₁₋₆ alkyl, C₃₋₆ cycloalkyl or 3-6 heterocyclyl is substituted with one or more G⁵;
R⁹ is independently selected from H, D, C₁₋₆ alkyl, C₃₋₆ naphthenic or 3-6-membered heterocyclic groups, and the alkyl, cycloalkyl and heterocyclyl are optionally substituted with one or more G⁶;
each of G¹, G², G³, G⁴, G⁵ and G⁶ is independently selected from H, D, cyano, halogen, C₁₋₆ alkyl, C₂₋₆ alkenyl, C₂₋₆ alkynyl, C₃₋₈ cycloalkyl or 3-8 membered heterocyclyl, C₆₋₁₀ aryl, 5-10 membered heteroaryl, —OR¹⁰, —OC(O)NR¹⁰R¹¹, —C(O)OR¹⁰, —C(O)NR¹⁰R¹¹, —C(O)R¹⁰, —NR¹⁰R¹¹, —NR¹⁰C(O)R¹¹, —NR¹⁰C(O)NR¹¹R¹², —S(O)ₘR¹⁰ or —NR¹⁰S(O)ₘR¹¹; and the alkyl, alkenyl, alkynyl, cycloalkyl, heteroalkyl, aryl or heteroaryl is independently substituted with substituents of one or more cyano, halogen, C₁₋₆alkyl, C₂₋₆alkenyl, C₂₋₆ alkynyl, C₃₋₈ heteroalkyl or 3-8 membered heterocyclyl, C₆₋₁₀ aryl, 5-10 membered heteroaryl, —OR¹³, —OC(O)NR¹³R¹⁴, —C(O)OR¹³, —C(O)NR¹³R¹⁴, —C(O)R¹³, —NR¹³R¹⁴, —NR¹³C(O)R¹⁴, —NR¹³C(O)NR¹⁴R¹⁵, —S(O)ₘR¹³ or —NR¹³S(O)ₘR¹⁴;
each of R⁷, R⁸, R⁹, R¹⁰, R¹¹, R¹², R¹³, R¹⁴ and R¹⁵ is independently selected from H, D, cyano, halogen, C₁₋₆ alkyl, C₃₋₈ cyclyl alkyl or 3-8 membered monocyclic heterocyclyl, monocyclic heteroaryl or phenyl;
m is 1 or 2; and
at each occurrence, each Ar is independently selected from

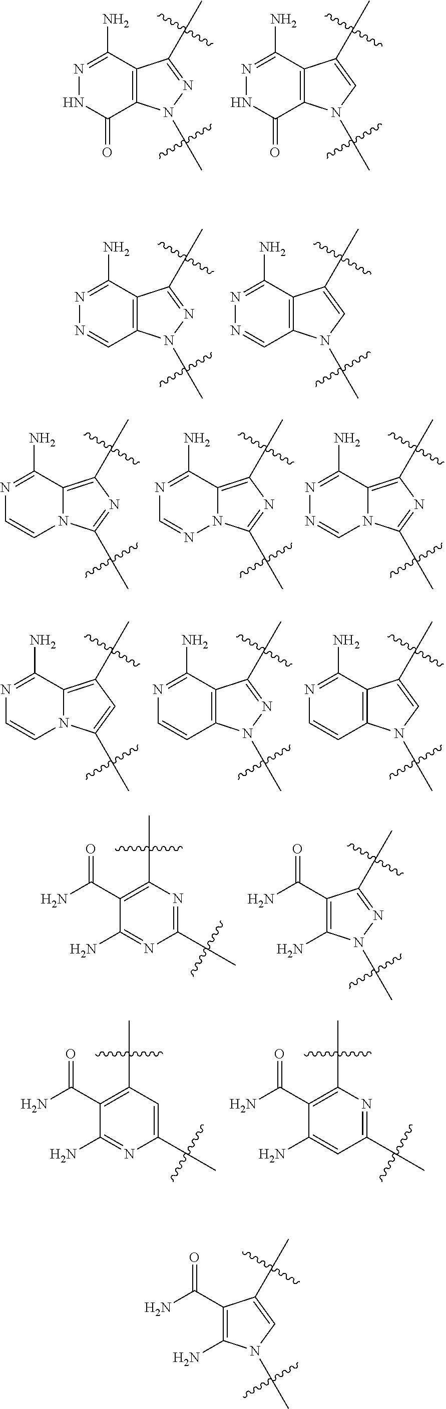

5. The compound represented by general formula (I), the pharmaceutically acceptable salt thereof or the stereoisomer thereof according to claim 1, wherein the compound is selected from:

| code | Chemical Name |
|---|---|
| 1 | (S)-1-(3-((4-((3,5-dimethoxyphenyl)ethynyl)pyrimidin-2-yl)amino)pyrrolidin-1- |
| | yl)prop-2-en-1-one |
| 2 | Preparation of (S)-1-(3-((5-bromo-4-((3,5-dimethoxyphenyl)ethynyl)pyrimidin- |
| | 2-yl)amino)pyrrolidin-1-yl)prop-2-en-1-one |
| 3 | (S)-1-(3-((4-((3,5-dimethoxyphenyl)ethynyl)-5-fluoropyrimidin-2- |
| | yl)amino)pyrrolidin-1-yl)prop-2-en-1-one |
| 4 | (S)-1-(3-((4-((3,5-dimethoxyphenyl)ethynyl)-5-(trifluoromethyl)pyrimidin-2- |
| | yl)amino)pyrrolidin-1-yl)prop-2-en-1-one |
| 5 | (S)-2-((1-acryloylpyrrolidin-3-yl)amino)-4-((3,5- |
| | dimethoxyphenyl)ethynyl)pyrimidine-5-carbonitrile |
| 6 | (S)-2-((1-acryloylpyrrolidin-3-yl)amino)-4-amino-6-((3,5- |
| | dimethoxyphenyl)ethynyl)pyrimidine-5-carboxamide |
| 7 | 2-((2-acryloyl-2-azaspiro[3.3]heptan-6-yl)amino)-4-amino-6-((3,5- |
| | dimethoxyphenyl)ethynyl)pyrimidine-5-carboxamide |
| 8 | 2-((6-acryloyl-6-azaspiro[3.4]octan-2-yl)amino)-4-amino-6-((3,5- |
| | dimethoxyphenyl)ethynyl)pyrimidine-5-carboxamide |
| 9 | 2-((7-acryloyl-7-azaspiro[3.5]nonan-2-yl)amino)-4-amino-6-((3,5- |
| | dimethoxyphenyl)ethynyl)pyrimidine-5-carboxamide |
| 10 | 2-((1-acryloylpiperidin-4-yl)amino)-4-amino-6-((3,5- |
| | dimethoxyphenyl)ethynyl)pyrimidine-5-carboxamide |
| 11 | (S)-2-((1-acryloylpiperidin-3-yl)amino)-4-amino-6-((3,5- |
| | dimethoxyphenyl)ethynyl)pyrimidine-5-carboxamide |
| 12 | (S)-4-amino-2-((1-(but-2-ynoyl)pyrrolidin-3-yl)amino)-6-((3,5- |
| | dimethoxyphenyl)ethynyl)pyrimidine-5-carboxamide |
| 13 | (S)-1-(3-((5-bromo-4-((3,5-dimethoxyphenyl)ethynyl)pyrimidin-2- |
| | yl)amino)pyrrolidin-1-yl)but-2-yn-1-one |
| 14 | (S)-1-(3-((4-((3,5-dimethoxyphenyl)ethynyl)-5-(trifluoromethyl)pyrimidin-2- |
| | yl)amino)pyrrolidin-1-yl)but-2-yn-1-one |
| 15 | (R)-2-((1-acryloylpyrrolidin-3-yl)amino)-4-amino-6-((3,5- |
| | dimethoxyphenyl)ethynyl)pyrimidine-5-carboxamide |
| 16 | (S)-2-((1-acryloylpyrrolidin-3-yl)amino)-4-((3,5- |
| | dimethoxyphenyl)ethynyl)pyrimidine-5-carboxamide |
| 17 | (S)-2-(3-acrylamidopyrrolidin-1-yl)-4-amino-6-((3,5- |
| | dimethoxyphenyl)ethynyl)pyrimidine-5-carboxamide |
| 18 | 2-(1-acryloylpyrrolidin-3-yl)-4-amino-6-((3,5- |
| | dimethoxyphenyl)ethynyl)pyrimidine-5-carboxamide |
| 19 | (R)-2-(1-acrylamidopyrrolidin-3-yl)-4-amino-6-((3,5- |
| | dimethoxyphenyl)ethynyl)pyrimidine-5-carboxamide |
| 20 | (S)-1-(1-acryloylpiperidin-3-yl)-4-amino-3-((3,5-dimethoxyphenyl)ethynyl)-1,6- |
| | dihydro-7H-pyrrolo[2,3-d]pyridazin-7-one |
| 21 | (S)-4-amino-1-(1-(but-2-ynoyl)piperidin-3-yl)-3-((3,5- |
| | dimethoxyphenyl)ethynyl)-1,6-dihydro-7H-pyrrolo[2,3-d]pyridazin-7-one |
| 22 | (S)-1-(1-acryloylpyrrolidin-3-yl)-4-amino-3-((3,5-dimethoxyphenyl)ethynyl)- |
| | 1,6-dihydro-7H-pyrrolo[2,3-d]pyridazin-7-one |
| 23 | (S)-4-amino-1-(1-(but-2-ynoyl)pyrrolidin-3-yl)-3-((3,5- |
| | dimethoxyphenyl)ethynyl)-1,6-dihydro-7H-pyrrolo[2,3-d]pyridazin-7-one |
| 24 | (S)-1-(1-acryloylpiperidin-3-yl)-4-amino-3-((3,5-dimethoxyphenyl)ethynyl)-1,6- |
| | dihydro-7H-pyrazolo[3,4-d]pyridazin-7-one |
| 25 | (S)-4-amino-1-(1-(but-2-ynoyl)piperidin-3-yl)-3-((3,5- |
| | dimethoxyphenyl)ethynyl)-1,6-dihydro-7H-pyrazolo[3,4-d]pyridazin-7-one |
| 26 | (S)-1-(1-acryloylpyrrolidin-3-yl)-4-amino-3-((3,5-dimethoxyphenyl)ethynyl)- |
| | 1,6-dihydro-7H-pyrazolo[3,4-d]pyridazin-7-one |
| 27 | (S)-4-amino-1-(1-(but-2-ynoyl)pyrrolidin-3-yl)-3-((3,5- |
| | dimethoxyphenyl)ethynyl)-1,6-dihydro-7H-pyrazolo[3,4-d]pyridazin-7-one |
| 28 | (S)-1-(1-acryloylpyrrolidin-3-yl)-4-amino-3-((2,6-difluoro-3,5- |
| | dimethoxyphenyl)ethynyl)-1,6-dihydro-7H-pyrazolo[3,4-d]pyridazin-7-one |
| 29 | (S)-4-amino-1-(1-(but-2-ynoyl)pyrrolidin-3-yl)-3-((2,6-difluoro-3,5- |
| | dimethoxyphenyl)ethynyl)-1,6-dihydro-7H-pyrazolo[3,4-d]pyridazin-7-one |
| 30 | (S)-1-(1-acryloylpyrrolidin-3-yl)-4-amino-3-((2,6-dichloro-3,5- |
| | dimethoxyphenyl)ethynyl)-1,6-dihydro-7H-pyrazolo[3,4-d]pyridazin-7-one |
| 31 | (S)-4-amino-1-(1-(but-2-ynoyl)pyrrolidin-3-yl)-3-((2,6-dichloro-3,5- |
| | dimethoxyphenyl)ethynyl)-1,6-dihydro-7H-pyrazolo[3,4-d]pyridazin-7-one |
| 32 | (S)-1-(1-acryloylpyrrolidin-3-yl)-4-amino-3-((2,6-difluoro-3,5- |
| | dimethoxyphenyl)ethynyl)-1,6-dihydro-7H-pyrazolo[3,4-d]pyridazin-7-one |
| 33 | (S)-4-amino-1-(1-(but-2-ynoyl)pyrrolidin-3-yl)-3-((2,6-difluoro-3,5- |
| | dimethoxyphenyl)ethynyl)-1,6-dihydro-7H-pyrazolo[3,4-d]pyridazin-7-one |
| 34 | (S)-4-amino-1-(1-(but-2-ynoyl)pyrrolidin-3-yl)-3-((2,6-difluoro-3,5- |
| | dimethoxyphenyl)ethynyl)-1,6-dihydro-7H-pyrazolo[3,4-d]pyridazin-7-one |
| 35 | (S)-4-amino-1-(1-(but-2-ynoyl)pyrrolidin-3-yl)-3-((2,6-dichloro-3,5- |
| | dimethoxyphenyl)ethynyl)-1,6-dihydro-7H-pyrazolo[3,4-d]pyridazin-7-one |
| 36 | (S)-1-(1-acryloylpyrrolidin-3-yl)-4-amino-3-(7-methoxy-5- |
| | methylbenzo[b]thiophen-2-yl)-1,6-dihydro-7H-pyrazolo[3,4-d]pyridazin-7-one |
| 37 | (S)-4-amino-1-(1-(but-2-ynoyl)pyrrolidin-3-yl)-3-(7-methoxy-5- |
| | methylbenzo[b]thiophen-2-yl)-1,6-dihydro-7H-pyrazolo[3,4-d]pyridazin-7-one |
| 38 | (S)-1-(1-acryloylpyrrolidin-3-yl)-4-amino-3-(7-methoxy-5- |
| | methylbenzo[b]thiophen-2-yl)-1,6-dihydro-7H-pyrrolo[2,3-d]pyridazin-7-one |
| 39 | (S)-4-amino-1-(1-(but-2-ynoyl)pyrrolidin-3-yl)-3-(naphthalen-2-yl)-1,6-dihydro- |
| | 7H-pyrrolo[2,3-d]pyridazin-7-one |
| 40 | (S)-1-(1-acryloylpyrrolidin-3-yl)-4-amino-3-(1-methyl-1H-indol-2-yl)-1,6- |
| | dihydro-7H-pyrrolo[2,3-d]pyridazin-7-one |
| 41 | (S)-1-(1-acryloylpyrrolidin-3-yl)-4-amino-3-(benzofuran-2-yl)-1,6-dihydro-7H- |
| | pyrrolo[2,3-d]pyridazin-7-one |
| 42 | (S)-1-(1-acryloylpyrrolidin-3-yl)-4-amino-3-(1-methyl-1H-indol-3-yl)-1,6- |
| | dihydro-7H-pyrrolo[2,3-d]pyridazin-7-one |
| 43 | (S)-1-(1-acryloylpyrrolidin-3-yl)-4-amino-3-(naphthalen-2-yl)-1,6-dihydro-7H- |
| | pyrrolo[2,3-d]pyridazin-7-one |
| 44 | (S)-1-(3-(8-amino-1-(7-methoxy-5-methylbenzo[b]thiophen-2-yl)imidazo[1,5- |
| | alpyrazin-3-yl)pyrrolidin-1-yl)prop-2-en-1-one |
| 45 | (S)-1-(3-(8-amino-1-(7-methoxy-5-methylbenzo[b]thiophen-2-yl)imidazo[1,5- |
| | a]pyrazin-3-yl)pyrrolidin-1-yl)but-2-yn-1-one |
| 46 | (S)-1-(3-(4-amino-5-(7-methoxy-5-methylbenzo[b]thiophen-2-yl)imidazo[5,1- |
| | f][1,2,4]triazin-7-yl)pyrrolidin-1-yl)prop-2-en-1-one |
| 47 | (S)-1-(3-(4-amino-5-(7-methoxy-5-methylbenzo[b]thiophen-2-yl)imidazo[5,1- |
| | f][1,2,4]triazin-7-yl)pyrrolidin-1-yl)but-2-yn-1-one |
| 48 | 1-(1-acryloylazetidin-3-yl)-4-amino-3-(7-methoxy-5-methylbenzo[b]thiophen-2- |
| | yl)-1,6-dihydro-7H-pyrazolo[3,4-d]pyridazin-7-one |
| 49 | 1-(1-acryloylazetidin-3-yl)-4-amino-3-(7-methoxy-5-methylbenzo[b]thiophen-2- |
| | yl)-1,6-dihydro-7H-pyrrolo[2,3-d]pyridazin-7-one |
| 50 | 1-(3-(8-amino-1-(7-methoxy-5-methylbenzo[b]thiophen-2-yl)imidazo[1,5- |
| | alpyrazin-3-yl)azetidin-1-yl)prop-2-en-1-one |
| 51 | 1-(3-(4-amino-5-(7-methoxy-5-methylbenzo[b]thiophen-2-yl)imidazo[5,1- |
| | f][1,2,4]triazin-7-yl)azetidin-1-yl)prop-2-en-1-one |
| 52 | 2-(1-acryloylazetidin-3-yl)-4-amino-6-(7-methoxy-5-methylbenzo[b]thiophen-2- |
| | yl)pyrimidine-5-carboxamide |
| 53 | 1-(1-acryloylazetidin-3-yl)-5-amino-3-(7-methoxy-5-methylbenzo[b]thiophen-2- |
| | yl)-1H-pyrazole-4-carboxamide |
| 54 | (S)-2-(1-acryloylpyrrolidin-3-yl)-4-amino-6-(7-methoxy-5- |
| | methylbenzo[b]thiophen-2-yl)pyrimidine-5-carboxamide |
| 55 | 1-(1-acryloylpyrrolidin-3-yl)-5-amino-3-(7-methoxy-5-methylbenzo[b]thiophen- |
| | 2-yl)-1H-pyrazole-4-carboxamide |

or their prodrug, stable isotope derivatives, pharmaceutically acceptable salts, solvates, isomers and their mixture and forms.

6. A pharmaceutical composition, comprising the compound or prodrug thereof, stable isotope derivatives, pharmaceutically acceptable salts, solvates or polymorphs or isomers, and pharmaceutically acceptable carriers according to claim 1.

7. The use of compounds or prodrugs thereof, stable isotope derivatives, pharmaceutically acceptable salts, solvates or polymorphs or isomers in the preparation of drugs for the treatment of FGFR-mediated diseases according to claim 1.

8. The use according to claim 7, wherein the FGFR-mediated diseases are one or more of non-small cell lung cancer, esophageal cancer, melanoma, gastric cancer, multiple myeloma, liver cancer, cholangiocarcinoma, prostate cancer, skin cancer, ovarian cancer, endometrial cancer, cervical cancer, bladder cancer, breast cancer, colon cancer, gliomas, and rhabdomyosarcoma.
